(19) United States
(12) Patent Application Publication
(10) Pub. No.: US 2016/0209414 A1
(43) Pub. Date: Jul. 21, 2016

(54) COMPOSITIONS AND METHODS FOR THE DIAGNOSIS AND TREATMENT OF BREAST CANCER

(71) Applicant: University of Washington Through Its Center For Commercialization, Seattle, WA (US)

(72) Inventors: Mary L. Disis, Renton, WA (US); Sasha Stanton, Seattle, WA (US)

(21) Appl. No.: 14/935,313

(22) Filed: Nov. 6, 2015

Related U.S. Application Data
(60) Provisional application No. 62/076,797, filed on Nov. 7, 2014.

(51) Int. Cl. G01N 33/574; G01N 33/564; A61K 39/00

(52) U.S. Cl. CPC G01N 33/57415; A61K 39/0011; G01N 33/564; A61K 2039/53

(57) ABSTRACT

Disclosed herein include compositions and methods for prevention and/or treatment of breast cancer. Also described herein included methods of early detection of breast cancer as well as assessing a subject's risk for the development of breast cancer.

### CROSS-REFERENCE

This application claims the benefit of U.S. Provisional Application No. 62/076,797, filed Nov. 7, 2014, which is incorporated herein by reference.

### STATEMENT AS TO FEDERALLY SPONSORED RESEARCH

This invention was made with government support under Grant No. U01CA141539, awarded by the National Cancer Institute (NCI). The government has certain rights in the invention.

### SEQUENCE LISTING

The instant application contains a Sequence Listing which has been filed electronically in ASCII format and is hereby incorporated by reference in its entirety. Said ASCII copy, created on Nov. 1, 2015, is named 41299-783-201-seqlist.txt and is 51 Kilobytes in size.

### BACKGROUND

Cancer therapy has conventionally been accomplished by surgical reduction of a tumor mass and subsequent chemo- and/or radiotherapy. This strategy can reduce the tumor and, in less advanced stages, often results in complete remission. Unfortunately, the prognosis for more advanced tumors has changed little over the past 50 years and a significant proportion of cancer-related deaths are caused by subsequent metastases. New prophylactic and therapeutic treatments are needed to combat the increasing occurrence of cancer.

Over 1 million people are diagnosed with breast cancer each year worldwide and more than 400,000 people die of breast cancer each year. It is estimated that one in eight women will be diagnosed with breast cancer at some point in her lifetime. Preventing the development of breast cancer could have significant health and economic benefits for all individuals. Billions of dollars would be saved if people did not need to receive expensive cancer-related surveillance and therapeutic interventions.

### SUMMARY

Disclosed herein, in certain aspects, are methods and kits for an early detection of an individual at-risk for developing breast cancer. Also described herein are methods, vaccines, compositions, and kits for vaccination of an at-risk individual to prevent breast cancer development. Further described herein include methods, vaccines, compositions, and kits for treatment of an individual diagnosed with breast cancer. Additionally described herein include methods, vaccines, compositions, and kits for inducing, altering, or modulating an immune response such as a Th1 type immune response.

In certain aspects, described herein is a method for identifying a subject's risk for developing breast cancer, the method comprising (a) incubating a biological sample from the subject with at least a first probe, wherein the biological sample comprises an autoantibody selected from Otud6B, Pdhx, Stk39, or a combination thereof and the first probe comprises a first recombinant polypeptide comprising an antigen of Otud6B, Pdhx, Stk39, or a combination thereof; (b) forming a first autoantibody-probe complex comprising the autoantibody and the first probe of step a); (c) measuring the concentration of the first autoantibody-probe complex, thereby determining the concentration of the autoantibody; and (d) identifying the subject as at risk for developing breast cancer if the subject has an elevated concentration of the autoantibody relative to a control. The control can be a biological sample obtained from a subject who is not at risk for developing breast cancer. The first recombinant polypeptide can be a polypeptide comprising at least 70%, 80%, 85%, 90%, 95%, 99% or 100% sequence identity to SEQ ID NO: 1 (Otud6B). The method of claim 1, wherein the first recombinant polypeptide is a polypeptide comprising at least 70%, 80%, 85%, 90%, 95%, 99% or 100% sequence identity to SEQ ID NO: 3 (Pdhx). The first recombinant polypeptide can be a polypeptide comprising at least 70%, 80%, 85%, 90%, 95%, 99% or 100% sequence identity to SEQ ID NO: 5 (Stk39). The method can further comprise i) incubating the biological sample with at least a second probe, wherein the second probe is a secondary antibody; (ii) forming an autoantibody-probe-second probe complex; and (iii) measuring the concentration of the autoantibody-probe-second probe complex, thereby determining the concentration of the autoantibody. The method can further comprise (i) incubating the biological sample with at least a third probe, wherein the third probe comprises a second recombinant polypeptide comprising an antigen of Zfp238, Lgals8, or Vps35, or a combination thereof, (ii) forming a second autoantibody-probe complex comprising the autoantibody and the third probe of step i); and (iii) measuring the concentration of the second autoantibody-probe complex, thereby determining the concentration of an autoantibody of Zfp238, Lgals8, or Vps35, or a combination thereof. The second recombinant polypeptide can be a polypeptide comprising at least 70%, 80%, 85%, 90%, 95%, 99% or 100% sequence identity to SEQ ID NO: 8 (Zfp238). The second recombinant polypeptide can be a polypeptide comprising at least 70%, 80%, 85%, 90%, 95%, 99% or 100% sequence identity to SEQ ID NO: 10 (Lgals8). The second recombinant polypeptide can be a polypeptide comprising at least 70%, 80%, 85%, 90%, 95%, 99% or 100% sequence identity to SEQ ID NO: 12 (Vps35). The autoantibody can be an IgG or an IgM autoantibody. The subject can be at risk for developing ductal carcinoma in situ, lobular carcinoma in situ, invasive ductal carcinoma, infiltrating ductal carcinoma, inflammatory breast cancer, triple-negative breast cancer, paget disease of the nipple, phyllodes tumor, angiosarcoma, adenoid cystic carcinoma, adenocystic carcinoma, low-grade adenosquamous carcinoma, medullary carcinoma, mucinous carcinoma, colloid carcinoma, papillary carcinoma, tubular carcinoma, metaplastic carcinoma, spindle cell carcinoma, squamous carcinoma, micropapillary carcinoma, or mixed carcinoma. The subject can be at risk for developing invasive breast cancer. The subject can be at risk for developing ductal carcinoma in situ. The biological sample can be a serum sample. The method can further comprise administering to the subject a composition comprising (i) an isolated and purified plasmid comprising at least one nucleotide sequence encoding a polypeptide comprising at least 70% sequence identity to an epitope sequence of SEQ ID NO: 1 (Otud6B), SEQ ID NO: 3 (Pdhx), or SEQ ID NO: 5 (Stk39); or (ii) a nucleic acid polymer that hybridizes to a target sequence encoding Otud6B, Pdhx, or Stk39, wherein the nucleic acid polymer modulates the gene expression of the target sequence; thereby reducing the risk of or preventing breast cancer in the subject. The isolated and purified plasmid can comprise at least one nucleotide sequence encoding a polypeptide comprising at least 80%, 85%, 90%, 95%, 99%, or 100% sequence identity to an epitope sequence of SEQ ID NO: 1. The isolated and purified plasmid can comprise at least one nucleotide sequence encoding a polypeptide comprising at least 80%, 85%, 90%, 95%, 99%, or 100% sequence identity to an epitope sequence of SEQ ID NO: 3. The isolated and purified plasmid can comprise at least one nucleotide sequence encoding a polypeptide comprising at least 80%, 85%, 90%, 95%, 99%, or 100% sequence identity to an epitope sequence of SEQ ID NO: 5. The composition can further comprise an isolated and purified plasmid comprising at least one nucleotide sequence encoding a polypeptide comprising at least 70%, 80%, 85%, 90%, 95%, 99%, or 100% sequence identity to an epitope sequence of SEQ ID NO: 8 (Zfp238). The composition can further comprise an isolated and purified plasmid comprising at least one nucleotide sequence encoding a polypeptide comprising at least 70%, 80%, 85%, 90%, 95%, 99%, or 100% sequence identity to an epitope sequence of SEQ ID NO: 10 (Lgals8). The composition can further comprise an isolated and purified plasmid comprising at least one nucleotide sequence encoding a polypeptide comprising at least 70%, 80%, 85%, 90%, 95%, 99%, or 100% sequence identity to an epitope sequence of SEQ ID NO: 12 (Vps35). The composition can further comprise a nucleic acid polymer that hybridizes to a target sequence encoding Zfp238, Lgals8, or Vps35, wherein the nucleic acid polymer modulates the gene expression of the target sequence. The composition can be formulated for subcutaneous, intramuscular, or intradermal administration.

In some aspects, described herein is a method for identifying a subject's risk for developing breast cancer, the method comprising: (a) incubating a biological sample from the subject with a panel of probes, wherein the biological sample comprises at least one autoantibody selected from Otud6B, Pdhx, Stk39, or a combination thereof and each probe from the panel of probes comprises a recombinant polypeptide comprising an antigen of Otud6B, Pdhx, Stk39, or a combination thereof; (b) forming at least one autoantibody-probe complex comprising the at least one autoantibody and the probe of step a); (c) measuring the concentration of the at least one autoantibody-probe complex, thereby determining the concentration of the at least one autoantibody; and (d) identifying the subject as at risk for developing breast cancer if the subject has an elevated concentration of the at least one autoantibody relative to a control.

In certain aspects, described herein is a method of prevention or treatment of breast cancer, comprising administering to a subject in need thereof a composition comprising: (a) an isolated and purified plasmid comprising at least one nucleotide sequence encoding a polypeptide comprising at least 70% sequence identity to an epitope sequence of SEQ ID NO: 1, SEQ ID NO: 3, or SEQ ID NO: 5; or (b) a nucleic acid polymer that hybridizes to a target sequence encoding Otud6B, Pdhx, or Stk39, wherein the nucleic acid polymer modulates gene expression of the target sequence. The isolated and purified plasmid can comprise at least one nucleotide sequence encoding a polypeptide comprising at least 80%, 85%, 90%, 95%, 99%, or 100% sequence identity to an epitope sequence of SEQ ID NO: 1. The isolated and purified plasmid can comprise at least one nucleotide sequence encoding a polypeptide comprising at least 80%, 85%, 90%, 95%, 99%, or 100% sequence identity to an epitope sequence of SEQ ID NO: 3. The isolated and purified plasmid can comprise at least one nucleotide sequence encoding a polypeptide comprising at least 80%, 85%, 90%, 95%, 99%, or 100% sequence identity to an epitope sequence of SEQ ID NO: 5. The method of claim 26, wherein the composition further comprises an isolated and purified plasmid comprising at least one nucleotide sequence encoding a polypeptide comprising at least 70%, 80%, 85%, 90%, 95%, 99%, or 100% sequence identity to an epitope sequence of SEQ ID NO: 8 (Zfp238). The composition can further comprise an isolated and purified plasmid comprising at least one nucleotide sequence encoding a polypeptide comprising at least 70%, 80%, 85%, 90%, 95%, 99%, or 100% sequence identity to an epitope sequence of SEQ ID NO: 10 (Lgals8). The composition can further comprise an isolated and purified plasmid comprising at least one nucleotide sequence encoding a polypeptide comprising at least 70%, 80%, 85%, 90%, 95%, 99%, or 100% sequence identity to an epitope sequence of SEQ ID NO: 12 (Vps35). The administration of the composition can comprise the isolated and purified plasmid reduces or inhibits tumor growth. The nucleic acid polymer can decrease gene expression of the target sequence. A decrease in the gene expression of the target sequence can lead to an increase in apoptosis of tumor cells. The composition can further comprise a nucleic acid polymer that hybridizes to a target sequence encoding Zfp238, Lgals8, or Vps35, wherein the nucleic acid polymer modulates the gene expression of the target sequence. The breast cancer can be ductal carcinoma in situ, lobular carcinoma in situ, invasive ductal carcinoma, infiltrating ductal carcinoma, inflammatory breast cancer, triple-negative breast cancer, paget disease of the nipple, phyllodes tumor, angiosarcoma, adenoid cystic carcinoma, adenocystic carcinoma, low-grade adenosquamous carcinoma, medullary carcinoma, mucinous carcinoma, colloid carcinoma, papillary carcinoma, tubular carcinoma, metaplastic carcinoma, spindle cell carcinoma, squamous carcinoma, micropapillary carcinoma, or mixed carcinoma. The subject can have invasive breast cancer. The subject can have ductal carcinoma in situ. The composition can be formulated for subcutaneous, intramuscular, or intradermal administration. The composition can be administered in combination with an additional therapeutic agent. The additional therapeutic agent can comprise chemotherapeutic agent, steroid, immunotherapeutic agent, targeted therapy, or a combination thereof. The additional therapeutic agent can comprise checkpoint inhibitors, costimulatory molecules, immune-cellular, or -intracellular targeting molecules, or combinations thereof.

In certain aspects, described herein is a composition comprising: (a) an isolated and purified plasmid comprising at least one nucleotide sequence encoding a polypeptide comprising at least 70% sequence identity to an epitope sequence of SEQ ID NO: 1, SEQ ID NO: 3, or SEQ ID NO: 5; and (b) an excipient and/or a carrier. The isolated and purified plasmid can comprise at least one nucleotide sequence encoding a polypeptide comprising at least 80%, 85%, 90%, 95%, 99%, or 100% sequence identity to an epitope sequence of SEQ ID NO: 1. The isolated and purified plasmid can comprise at least one nucleotide sequence encoding a polypeptide comprising at least 80%, 85%, 90%, 95%, 99%, or 100% sequence identity to an epitope sequence of SEQ ID NO: 3. The isolated and purified plasmid can comprise at least one nucleotide sequence encoding a polypeptide comprising at least 80%, 85%, 90%, 95%, 99%, or 100% sequence identity to an epitope sequence of SEQ ID NO: 5. The composition can further comprise an isolated and purified plasmid comprising at least one nucleotide sequence encoding a polypeptide comprising at least 70%, 80%, 85%, 90%, 95%, 99%, or 100% sequence identity to an epitope sequence of SEQ ID NO: 8 (Zfp238). The composition can further comprise an isolated and purified plasmid comprising at least one nucleotide sequence encoding a polypeptide comprising at least 70%, 80%, 85%, 90%, 95%, 99%, or 100% sequence identity to an epitope sequence of SEQ ID NO: 10 (Lgals8). The composition can further comprise an isolated and purified plasmid comprising at least one nucleotide sequence encoding a polypeptide comprising at least 70%, 80%, 85%, 90%, 95%, 99%, or 100% sequence identity to an epitope sequence of SEQ ID NO: 12 (Vps35). The plasmid can be an expression vector. The expression vector can be pBK-CMV. The composition can further comprise an adjuvant. The composition can be formulated for subcutaneous, intramuscular, or intradermal administration.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various aspects of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates vaccination with individual pre-diagnostic tumor antigens but not with established tumor antigens inhibits tumor growth. FIG. 1A shows six pre-diagnostic tumor antigens which are recovered from the SEREX screen of pre-diagnostic TgMMTV-neu mice. FIG. 1B shows TgMMTV-neu mice challenged with implanted 3×10⁵ MMC tumor cells after 3 vaccinations q14d with either the pre-diagnostic tumor antigens or vector control. FIG. 1C shows three established tumors recovered from SEREX screen of TgMMTV-neu mice with established tumors (adapted Lu et al Cancer Research 2006, 1). FIG. 1D shows TgMMTV-neu mice (n=3) challenged with 3×10⁵ MMC tumor cells after 3 vaccinations q14d with either the established tumor antigens, positive control irradiated MMC tumor cells, or PBS. *** p<0.001, NS no statistical difference (personal communication H. Lu).

FIG. 2 illustrates pre-diagnostic tumor autoantibodies that are elevated in the serum of TgMMTV-neu mice prior to tumor development and can discriminate mice that will develop tumors. FIG. 2A shows pre-diagnostic tumor autoantibodies Pdhx (Panel i), Otud6b (Panel ii), and Stk39 (Panel iii), which are detectable in mice prior to developing palpable tumor, IgG (), IgM (⋄) antibody level and tumor growth (▪) measured in each animal with specific antibody response to antigen, IgG ( ) and IgM ( [symbol] ) measured over time from control animals. Left Y axis: Tumor volume; right Y axis: antibody titer; X axis: mouse age (in weeks). Arrow shows the time point when tumor is palpable. * indicates p<0.05 from initial value. FIG. 2B shows panel of IgM and IgG autoantibodies to the pre-diagnostic tumor antigens comparing pre-diagnostic MMTV-neu mice verses tumor bearing mice (n=21 mice). With a panel of Otud6B, Stk39, and Lgals8 the area under the curve (AUC) was 0.924 (CI 0.81-1.0 p<0.001) with sensitivity of 0.85 and specificity of 0.9.

FIG. 3 shows pre-diagnostic tumor autoantibodies identified in mice that can discriminate women who will develop breast cancer from matched controls. FIG. 3A shows panel of IgM and IgG autoantibodies to early tumor antigens in pre-diagnostic sera from women who would develop breast cancer in over 150 days (n=48) verses women who would develop breast cancer within 150 days (n=46) from the WHI study. With the panel of PDHX, OTUD6B, and STK39 over 150 days prior to diagnosis of malignancy the AUC was 0.68 (CI 0.565-0.787 p=0.003) with sensitivity of 67% and specificity of 65%. FIG. 3B shows comparison of AUC of a panel of PDHX, OTUD6B, and STK39 IgG and IgM autoantibodies in women diagnosed with breast cancer more or less than 150 days prior to diagnosis.

FIG. 4 shows vaccination with a panel of pre-diagnostic tumor antigens but not a panel of established tumor antigens inhibits tumor growth. Spontaneous tumor growth at 37 weeks demonstrates 34.8% decreased tumor volume as compared to vector vaccinated mice (*p=0.02). The established antigen vaccinated mice have 31.3% more growth than the pre-diagnostic antigen vaccinated mice (** p=0.005) where the growth between the established tumor and vector-vaccinated mice is not statistically different (p=0.69, NS).

FIG. 5 shows pre-diagnostic autoantibodies identified in mice can discriminate women who will develop breast cancer from matched controls. FIG. 5A illustrates panel of IgM and IgG autoantibodies to early tumor antigens in pre-diagnostic sera from women who would develop breast cancer in over 150 days (n=48) verses women who would develop breast cancer within 150 days (n=46) from the WHI study. With the panel of PDHX, OTUD6B, and STK39 over 150 days prior to diagnosis of malignancy the AUC was 0.68 (CI 0.565-0.787 p=0.003) with sensitivity of 67% and specificity of 65%. FIG. 5B shows a comparison of AUC of a panel of PDHX, OTUD6B, and STK39 IgG and IgM autoantibodies in women diagnosed with breast cancer more or less than 150 days prior to diagnosis.

FIG. 6 shows combining pre-diagnostic autoantibodies with antibodies directed against established tumor antigens improves AUC over individual panels. FIG. 6A shows expression of established tumor antigens HER2, P53, and CYCB1 in pre-diagnostic breast cancer sera from the WHI study, figure adapted from Lu et al Cancer Prevention Research 2012 (3). FIG. 6B shows panel 1 (Pre-diagnostic tumor antigens PDHX, OTUD6B, and STK39) had similar AUC from women >150 days prior to breast diagnosis as Panel 2 (published tumor antigens HER2, p53, and Cyclin B1). When both panels are evaluated together there is an additive effect with AUC 0.75.

FIG. 7 shows murine pre-diagnostic antigenic proteins are expressed in human ductal carcinoma in situ and invasive breast cancer. Gene expression relative to 1 actin for the early tumor antigen proteins in normal breast (n=6), DCIS (n=31) and IDC (n=36). Dashed line: mean +2 SD above normal breast tissue. * p<0.05.

FIG. 8 shows tumor-associated autoantibodies detected in the sera of TgMMTV-neu mice prior to the development of palpable disease. FIG. 8A shows pre-diagnostic tumor autoantibody Lgals8 as detectable in mice prior to developing palpable tumor. FIG. 8B shows pre-diagnostic tumor autoantibody Vps35 as detectable in mice prior to developing palpable tumor. FIG. 8C shows pre-diagnostic tumor autoantibody Znf238 as detectable in mice prior to developing palpable tumor. IgG (), IgM (⋄) antibody level and tumor growth (▪) measured in each animal with specific antibody response to antigen. IgG ( ) and IgM ( [symbol] ) measured over time from control animals. Left Y axis: Tumor volume; right Y axis: antibody titer; X axis: mouse age (in weeks). Arrow indicates first palpable tumor. * indicates p<0.05 from initial value.

FIG. 9 shows IgM antibodies to pre-diagnostic antigens as more commonly identified in sera from mice prior to the development of palpable breast cancer. Incidence of IgG (black bar) and IgM (white bar) for antigen Pdhx, Otud6b, Stk39, Lgals8, Znf238 and Vps35 in sera obtained (FIG. 9A) pre-diagnosis or when (FIG. 9B) tumor bearing. Incidence of IgG (black bar) and IgM (white bar) antibody detection for a panel with any 1 antigen, 2 antigens, 3 antigens and 4 antigens in sera obtained (FIG. 9C) pre-diagnosis or when (FIG. 9D) tumor bearing.

FIG. 10 shows tumor growth inhibition in TgMMTV-neu mice vaccinated with pre-diagnostic tumor antigens. TgMMTV-neu mice (n=5) were vaccinated three times in q14 day intervals with plasmids containing either Otud6b, Stk39, or Pdhx and then had 3×10⁵ MMC tumor cells implanted on day 0. Tumor growth inhibition shown over time since tumor implant. *** p<0.001.

FIG. 11 shows immune depletion of T cells but not B cells inhibiting vaccine protective effect on tumor growth. TgMMTV-neu mice (n=3 per group) were vaccinated with plasmid DNA encoding tumor antigens (FIG. 11A) Stk39, (FIG. 11B) Pdhx, and (FIG. 11C) Otud6B approximately every 14 days for 3 vaccinations and then given 3×10⁵ syngenic MMC cells subcutaneously on day 0. Depletion of CD3 (T cell), CD22 (B cell), or control IgG were performed starting 3 days before tumor implants. *** p<0.001 as compared to Vector+IgG (circles). NS no significant difference.

FIG. 12 illustrates knocking down expression of the pre-diagnostic antigens leading to decreased survival and increased apoptosis. Using four pooled siRNA for each target in the syngenic MMC mouse tumor cell line the pre-diagnostic (grey bars) antigens all have <60% survival and >1.5 fold increase in apoptosis. The siRNA knockdown was compared to mock transfected (mock), a non-targeting control (neg), and untransfected control (untx) (white bars) and positive apoptosis control (black bars). * p<0.05 ** p<0.01 *** p<0.001 **** p<0.0001.

FIG. 13 shows pooled targeted siRNA which knocked down expression of each pre-diagnostic target. MMC cells were either mock transfected (white bars) or transfected with four pooled siRNA for each of the pre-diagnostic target (grey bars) and amplified with specific primers using RT PCR. * p<0.05 **p<0.01 *** p<0.001 **** p<0.0001 reduced expression from mock.

### DETAILED DESCRIPTION

Breast cancer is immunogenic and breast tumor proteins have been shown to stimulate both antibody and T cell immune response. In some instances, breast cancer antigens identified in the literature and the Cancer Immune Database are from cancer patients with established tumors (e.g. HER2, MUC1, hTERT, CEA, etc.). In some cases, vaccines from the established tumor antigens have not shown clinical efficacy. It is proposed that in these cases, the lack of clinical efficacy can be due to the tumor antigens as not being antigens associated with early tumorigenesis.

Described herein are pre-diagnostic tumor antigens of breast cancer. The disclosure also provides compositions and methods that utilize pre-diagnostic tumor antigen for the prevention and/or treatment of breast cancer. The disclosure further provides methods of early detection (e.g., non-invasive screening) of women at risk (e.g., high risk) for developing breast cancer.

In some aspects, the disclosure provides compositions that comprise an isolated and purified plasmid comprising at least one nucleotide sequence encoding a polypeptide comprising at least 70% sequence identity to an epitope sequence of Otud6B, Pdhx, or Stk39; and an excipient and/or a carrier.

In additional aspects, the disclosure provides compositions that comprise a nucleic acid polymer that hybridizes to a target sequence encoding Otud6B, Pdhx, or Stk39, wherein the nucleic acid polymer modulates gene expression of the target sequence.

In other aspects, the disclosure provides a method of prevention or treatment of breast cancer, comprising administering to a subject in need thereof a composition that comprises an isolated and purified plasmid comprising at least one nucleotide sequence encoding a polypeptide comprising at least 70% sequence identity to an epitope sequence of Otud6B, Pdhx, or Stk39; or a nucleic acid polymer that hybridizes to a target sequence encoding Otud6B, Pdhx, or Stk39, wherein the nucleic acid polymer modulates gene expression of the target sequence.

Additionally, the disclosure provides a method of identifying a subject's risk for developing breast cancer, in which the method comprises a) incubating a biological sample from the subject with at least a first probe, wherein the biological sample comprises an autoantibody selected from Otud6B, Pdhx, Stk39, or a combination thereof and the first probe comprises a recombinant polypeptide comprising an antigen of Otud6B, Pdhx, Stk39, or a combination thereof; b) forming a first autoantibody-probe complex comprising the autoantibody and the first probe of step a); c) measuring the concentration of the first autoantibody-probe complex, thereby determining the concentration of the autoantibody; and d) identifying the subject as at risk for developing breast cancer if the subject has an elevated concentration of the autoantibody relative to a control.

### Identification of Antigens

The compositions and methods described herein include the identification and engineering of pre-diagnostic breast cancer antigens in a pharmaceutical composition (e.g., a vaccine) and include the detection of autoantibodies that recognize the pre-diagnostic breast cancer antigens for the early detection of individuals at risk for developing breast cancer. While any techniques known to one of ordinary skill in the art may be used to identify antigens expressed by a subject with or without breast cancer, in an exemplary case, suitable antigens may be identified using the methods described herein. In some cases, the methods may include screening sera from subjects to obtain the concentration levels of one or more antigens and compare the concentration levels of the antigens with those of a control. In some cases, the screening may be antibody screening. For example, the antibodies screened may be IgG or IgM antibodies. In some cases, the sera may be from a subject with breast cancer. In other cases, the sera may be from a subject who does not have breast cancer. The control may be the concentration levels of the antigens obtained from the sera of a subject who does not have breast cancer.

Cancer antigens described herein may be a portion of a protein or polypeptide. In some cases, the portion may be a percentage of a protein or polypeptide. In some cases, the percentage may be less than 1%, 5%, 10%, 15%, 20%, 25%, 30%, 35%, 40%, 45%, 50%, 55%, 60%, 65%, 70%, 75%, 80%, 85%, 90%, 95% or 100% of a protein or polypeptide. In some cases, the portion may be located at the C terminus of a protein or polypeptide. In other cases, the portion may be located near the C terminus of a protein or polypeptide. For example, near the C terminus may be within 1%, 5%, 10%, 15%, 20%, 25%, 30%, 35%, 40%, 45%, or 50% of the length of the total protein or polypeptide from the median. In some cases, the portion may be located at the N terminus of a protein or polypeptide. In other cases, the portion may be located near the N terminus of a protein or polypeptide. For example, near the N terminus may be within 1%, 5%, 10%, 15%, 20%, 25%, 30%, 35%, 40%, 45%, or 50% of the length of the total protein or polypeptide from the median. In some cases, the portion may be located near the middle of a protein or polypeptide. In other cases, the portion may be located near the middle of a protein or polypeptide. For example, near the middle may be within 1%, 5%, 10%, 15%, 20%, 25%, 30%, 35%, 40%, 45%, or 50% of the length of the total protein or polypeptide from the termini.

At least one antigen may be identified and screened for suitability as an antigen in a composition described herein (e.g., a vaccine). In some cases, one antigen may be identified and screened. In other cases, more than one antigen may be identified and screened, more than two antigens may be identified and screened, more than three antigens may be identified and screened, more than four antigens may be identified and screened, more than five antigens may be identified and screened, more than six antigens may be identified and screened, more than seven antigens may be identified and screened, more than eight antigens may be identified and screened, more than nine antigens may be identified and screened, more than ten antigens may be identified and screened, more than 11 antigens may be identified and screened, more than 12 antigens may be identified and screened, more than 13 antigens may be identified and screened, more than 14 antigens may be identified and screened, more than 15 antigens may be identified and screened, more than 20 antigens may be identified and screened, more than 25 antigens may be identified and screened, more than 30 antigens may be identified and screened, more than 35 antigens may be identified and screened, more than 40 antigens may be identified and screened, more than 45 antigens may be identified and screened or more than 50 antigens may be identified and screened for suitability in a vaccine. In an exemplary case, six antigens may be identified and screened for suitability in a vaccine. In a second exemplary case, three antigens may be identified and screened for suitability in a vaccine.

The antigens screened for suitability in a vaccine may be derived from any protein detected in the sera from a subject with or without breast cancer using the screening techniques known to one of ordinary skill in the art. The screening may be antibody screening. Often, the antigens may be immunogenic in both breast cancer subjects and subjects without breast cancer.

### Mapping Epitopes of Antigens

The compositions and methods provided herein include mapping of at least one epitope within the antigens, such that the epitopes result in a T cell mediated response when administered to a subject. The T cell mediated response can be a Th1 immune response or a Th2 immune response. The epitope may be a portion of an antigen (e.g., identified above). For example, the epitope may be a peptide of an antigenic protein and/or a portion of an antigenic protein.

In some cases, the epitopes may be human leukocyte antigen (HLA) class I epitopes derived from breast cancer antigens. For example, HLA class I epitopes may include epitopes which bind to HLA-A, -B, and -C molecules. In some cases, the epitopes may be class II epitopes derived from breast cancer antigens for cancer vaccine development. For example, HLA class II epitopes may include epitopes which bind to HLA-DP, -DM, -DOA, -DOB, -DQ and -DR molecules. In some cases, in addition to the methods described herein, epitopes may be mapped using the steps of, (1) determining if the epitopes bind MHC (e.g., with high affinity) by at least one HLA allele (e.g., HLA-DR, i.e., are universal epitopes), (2) determining the cytokines (e.g., IFNγ or IL-10) secreted by the epitopes, and (3) determining whether T-cells may recognize peptides (e.g., epitopes) processed by antigen presenting cells (APCs), i.e., are native epitopes. In some cases, T-cell lines may be used. For example, T-cell lines may be epitope-derived T-cell lines. In some cases, the T-cell may be an exogenous T-cell engineered to express a Chimeric Antigen Receptor construct that binds the epitope with high selectivity and avidity. In some cases, the epitopes may be derived from proteins (e.g., recombinant proteins). In other cases, the proteins may be native proteins. In some cases, the proteins may be processed endogenously. In other cases, the proteins may be processed exogenously. In some cases, the proteins may be processed endogenously by autologous APCs. In other cases, the proteins may be processed exogenously by autologous APCs.

In all cases, the peptides are epitopes mapped from antigens and may be identified using the methods described herein for the selection of peptide epitopes. In some cases, the epitopes may be derived from human proteins that may be used directly in a peptide based vaccine. In other cases, the epitopes may be derived from human proteins and the encoding nucleic acid sequences may be incorporated into a nucleic acid construct designed to induce expression of the epitope in the subject following administration. For example, the nucleic acid construct may allow for the immune response to at least one epitope to be entrained, amplified, attenuated, suppressed, or eliminated to specific sets of self-proteins. In some cases, the peptide or the nucleic acid construct may be optimized into a protein or plasmid-based vaccination to induce, amplify or entrain a T cell mediated response. In some cases, the peptide or the nucleic acid construct may be optimized into a protein or plasmid-based vaccination to suppress, attenuate or eliminate a pathological response, in a subject (e.g., human or animal) in need thereof.

In some cases, the peptides are located within portions of a protein or polypeptide such that the protein or polypeptide stimulates secretion of a Th1 type cytokine. Exemplary Th1 type cytokines include IFNγ, TNF-α, IL-2, and IL-10. In some cases, the peptides are located within portions of a protein or polypeptide such that the protein or polypeptide stimulates secretion of a Th2 type cytokine. Exemplary Th2 type cytokines include IL-4, IL-5, IL-6, IL-9, IL-10, and IL-13. In some cases, the peptides are located within portions of a protein or polypeptide such that the protein or polypeptide inhibits secretion of a Th1 type cytokine, a Th2 type cytokine, or a combination thereof. In additional cases, the peptide may further stimulate secretion of a Th1 type cytokine, inhibits secretion of a Th2 type cytokine, or vice versa.

In some cases, the amino acids comprising the peptide may be tuned such that the desired effect of the peptide on Th1 type secretion and/or the desired effect of the peptide on Th2 type secretion may be achieved. For example, a peptide which stimulates secretion of both IFNγ and IL-10 may be tuned such that the length of the peptide is shortened to eliminate amino acids which stimulate IL-10 secretion such that the peptide only stimulates secretion of IFNγ.

In some cases, identified epitopes may be included in vaccine compositions of extended epitope vaccines. In some cases, extended epitopes may be 40-80-mer peptides. In an exemplary case, either the nucleic acid sequences or the peptide sequences are juxtaposed for construction of extended epitope sequences. Juxtaposition (e.g., within 10 amino acids of each other) of selected peptides within the parent protein may allow for the construction of in-tandem extended epitopes that may contain tolerating and/or suppressive epitopes. For example, the in-tandem extended epitopes may contain short intervening, <10 amino acid sequences. Any of these peptides and/or extended epitopes (embodied either as the peptide itself, or as the corresponding nucleic acid construct) singularly, or in any combination, may be optimized into a protein or plasmid-based vaccination that will specifically induce, amplify or entrain a protective immune response, or alternatively, will suppress, attenuate or eliminate a pathological one, in a subject (human or animal) in need thereof.

In some cases, the epitopes may be a length of amino acids. In some cases, the epitopes may be less than five amino acids, less than 10 amino acids, less than 15 amino acids, less than 20 amino acids, less than 25 amino acids, less than 30 amino acids, less than 35 amino acids, less than 40 amino acids, less than 45 amino acids, less than 50 amino acids, less than 55 amino acids, less than 60 amino acids, less than 70 amino acids, less than 75 amino acids, less than 80 amino acids, less than 85 amino acids, less than 90 amino acids, less than 95 amino acids, less than 100 amino acids, less than 110 amino acids, less than 120 amino acids, less than 130 amino acids, less than 140 amino acids, less than 150 amino acids, less than 160 amino acids, less than 170 amino acids, less than 180 amino acids, less than 190 amino acids, less than 200 amino acids, less than 210 amino acids, less than 220 amino acids, less than 230 amino acids, less than 240 amino acids, less than 250 amino acids, less than 260 amino acids, less than 270 amino acids, less than 280 amino acids, less than 290 amino acids, less than 300 amino acids, less than 350 amino acids, less than 400 amino acids, less than 450 amino acids or less than 500 amino acids.

The epitopes used in the compositions and methods described herein can be used to generate a chimeric antigen receptor (CAR) T cell. The engineered T cell can express an antibody, such as a single chain variable fragment (scFv), and can recognize one or more of the epitopes described herein present on a breast cancer tumor cell. The expressed antibody can further induce an engineered immune response by the tumor cell. Sometimes, the one or more of the epitopes are selected from Otud6B, Pdhx, Stk39, Zfp238, Lgals8, or Vps35. The one or more of the epitopes can comprise at least 90%, at least 95%, or at least 99% sequence identity to at least 8 contiguous amino acids of SEQ ID NOs: 1-12. In some instances, the engineered T cell can express an antibody and can recognize one or more of the epitopes selected from Otud6B, Pdhx, Stk39, Zfp238, Lgals8, or Vps35 present on a breast cancer tumor cell. In additional instances, the engineered T cell can express an antibody and can recognize one or more of the epitopes comprising at least 90%, at least 95%, or at least 99% sequence identity to at least 8 contiguous amino acids of SEQ ID NOs: 1-12 present on a breast cancer tumor cell.

The epitopes used in the compositions and methods described herein can be used as suitable targets for engineered T cell receptors (TCRs). In some instances, the gene encoding the engineered T cell receptor is introduce into a T cell such as for example by a viral delivery method and subsequently expresses the engineered TCR. The engineered TCRs which can recognize one or more of the epitopes described herein can be used for engineered T Cell Receptor-based therapies including autologous and heterologous cell therapies. As disclosed above, the one or more of the epitopes are selected from Otud6B, Pdhx, Stk39, Zfp238, Lgals8, or Vps35. The one or more of the epitopes can comprise at least 90%, at least 95%, or at least 99% sequence identity to at least 8 contiguous amino acids of SEQ ID NOs: 1-12. In some instances, the engineered TCR can recognize one or more of the epitopes selected from Otud6B, Pdhx, Stk39, Zfp238, Lgals8, or Vps35. In additional instances, the engineered TCR can recognize one or more of the epitopes comprising at least 90%, at least 95%, or at least 99% sequence identity to at least 8 contiguous amino acids of SEQ ID NOs: 1-12.

The epitopes used in the compositions and methods described herein can be used in an array or in a format to serve as bait for capturing TCR or TCR constructs that bind to it with desired affinity properties. The epitopes can also be used as antigens or antigenic components of a construct for use in generating antibodies (e.g., monoclonal, polyclonal, fab, fab dimer, fv, scfv, diabody, nanobody, minibody, recombinant, veneer) that bind the epitope with desired affinity properties.

### Compositions Comprising Epitopes for a Breast Cancer Vaccine

The compositions described herein can include a composition that comprises an isolated and purified plasmid comprising at least one nucleotide sequence encoding a polypeptide comprising an epitope of a pre-diagnostic antigen of breast cancer. The compositions described herein can include a composition that comprises an isolated and purified plasmid comprising at least one nucleotide sequence encoding a polypeptide comprising Otud6B, Pdhx, or Stk39. The isolated and purified plasmid can comprise at least one nucleotide sequence encoding a polypeptide comprising at least 70% sequence identity to an epitope sequence of Otud6B, Pdhx, or Stk39. The isolated and purified plasmid can comprise at least one nucleotide sequence encoding a polypeptide comprising at least 80%, 85%, 90%, 95%, 99%, or 100% sequence identity to an epitope sequence of Otud6B. The isolated and purified plasmid can comprise at least one nucleotide sequence encoding a polypeptide consisting of Otud6B. The isolated and purified plasmid can comprise at least one nucleotide sequence encoding a polypeptide comprising at least 80%, 85%, 90%, 95%, 99%, or 100% sequence identity to an epitope sequence of Pdhx. The isolated and purified plasmid can comprise at least one nucleotide sequence encoding a polypeptide consisting of Pdhx. The isolated and purified plasmid can comprise at least one nucleotide sequence encoding a polypeptide comprising at least 80%, 85%, 90%, 95%, 99%, or 100% sequence identity to an epitope sequence of Stk39. The isolated and purified plasmid can comprise at least one nucleotide sequence encoding a polypeptide consisting of Stk39.

Otud6B can comprise the sequence as illustrated in SEQ ID NO: 1. Pdhx can comprise the sequence as illustrated in SEQ ID NO: 3. Stk39 can comprise the sequence as illustrated in SEQ ID NO: 5.

The isolated and purified plasmid can comprise at least one nucleotide sequence encoding a polypeptide comprising at least 70% sequence identity to an epitope sequence of SEQ ID NO: 1, SEQ ID NO: 3, or SEQ ID NO: 5. The isolated and purified plasmid can comprise at least one nucleotide sequence encoding a polypeptide comprising at least 80%, 85%, 90%, 95%, 99%, or 100% sequence identity to an epitope sequence of SEQ ID NO: 1. The isolated and purified plasmid can comprise at least one nucleotide sequence encoding a polypeptide consisting of SEQ ID NO: 1. The isolated and purified plasmid can comprise at least one nucleotide sequence encoding a polypeptide comprising at least 80%, 85%, 90%, 95%, 99%, or 100% sequence identity to an epitope sequence of SEQ ID NO: 3. The isolated and purified plasmid can comprise at least one nucleotide sequence encoding a polypeptide consisting of SEQ ID NO: 3. The isolated and purified plasmid can comprise at least one nucleotide sequence encoding a polypeptide comprising at least 80%, 85%, 90%, 95%, 99%, or 100% sequence identity to an epitope sequence of SEQ ID NO: 5. The isolated and purified plasmid can comprise at least one nucleotide sequence encoding a polypeptide consisting of SEQ ID NO: 5.

In some cases, the composition further comprises an isolated and purified plasmid comprising at least one nucleotide sequence encoding a polypeptide comprising an epitope sequence of Zfp238, Lgals8, or Vps35.

The isolated and purified plasmid can comprise at least one nucleotide sequence encoding a polypeptide comprising at least 70%, 80%, 85%, 90%, 95%, 99%, or 100% sequence identity to an epitope sequence of Zfp238. The isolated and purified plasmid can comprise at least one nucleotide sequence encoding a polypeptide consisting of Zfp238. The isolated and purified plasmid can comprise at least one nucleotide sequence encoding a polypeptide comprising at least 70%, 80%, 85%, 90%, 95%, 99%, or 100% sequence identity to an epitope sequence of Lgals8. The isolated and purified plasmid can comprise at least one nucleotide sequence encoding a polypeptide consisting of Lgals8. The isolated and purified plasmid can comprise at least one nucleotide sequence encoding a polypeptide comprising at least 70%, 80%, 85%, 90%, 95%, 99%, or 100% sequence identity to an epitope sequence of Vps35. The isolated and purified plasmid can comprise at least one nucleotide sequence encoding a polypeptide consisting of Vps35.

Zfp238 can comprise the sequence as illustrated in SEQ ID NO: 8, Lgals8 can comprise the sequence as illustrated in SEQ ID NO: 10. Vps35 can comprise the sequence as illustrated in SEQ ID NO: 12.

The isolated and purified plasmid can comprise at least one nucleotide sequence encoding a polypeptide comprising at least 70%, 80%, 85%, 90%, 95%, 99%, or 100% sequence identity to an epitope sequence of SEQ ID NO: 8 (Zfp238). The isolated and purified plasmid can comprise at least one nucleotide sequence encoding a polypeptide consisting of SEQ ID NO: 8 (Zfp238). The isolated and purified plasmid can comprise at least one nucleotide sequence encoding a polypeptide comprising at least 70%, 80%, 85%, 90%, 95%, 99%, or 100% sequence identity to an epitope sequence of SEQ ID NO: 10 (Lgals8). The isolated and purified plasmid can comprise at least one nucleotide sequence encoding a polypeptide consisting of SEQ ID NO: 10 (Lgals8). The isolated and purified plasmid can comprise at least one nucleotide sequence encoding a polypeptide comprising at least 70%, 80%, 85%, 90%, 95%, 99%, or 100% sequence identity to an epitope sequence of SEQ ID NO: 12 (Vps35). The isolated and purified plasmid can comprise at least one nucleotide sequence encoding a polypeptide consisting of SEQ ID NO: 12 (Vps35).

Administration of the composition that comprises the isolated and purified plasmid can elicit a T cell mediated immune response. The T cell mediated immune response can be a Th1 type response or a Th2 type response. Administration of the composition can elicit Th1 type cytokine secretions such as IFNγ, TNF-α, IL-2, and/or IL-10 secretion, Th2 type cytokine secretions such as IL-4, IL-5, IL-6, IL-9, IL-10, and/or IL-13, or a combination thereof. In some cases, the immune response can further be characterized by a ratio of Type I cytokine production to Type II cytokine production, such that the ratio is greater than 1. In other cases, the immune response is characterized by a ratio of Type I cytokine production to Type II cytokine production such that the ratio is less than 1.

The composition can further comprise an adjuvant and optionally a pharmaceutical carrier. In some cases, the adjuvant is GM-CSF.

The composition can be administered to a subject to elicit an immune response in a subject. In some cases, the composition is effective to eliminate a number of cells associated with breast cancer in a subject. Administration of the composition may prevent the growth of cells associated with breast cancer. Administration of the composition may further reduce or inhibit tumor growth in a subject.

Exemplary breast cancer subtypes can include ductal carcinoma in situ, lobular carcinoma in situ, invasive ductal carcinoma, infiltrating ductal carcinoma, inflammatory breast cancer, triple-negative breast cancer, paget disease of the nipple, phyllodes tumor, angiosarcoma, adenoid cystic carcinoma, adenocystic carcinoma, low-grade adenosquamous carcinoma, medullary carcinoma, mucinous carcinoma, colloid carcinoma, papillary carcinoma, tubular carcinoma, metaplastic carcinoma, spindle cell carcinoma, squamous carcinoma, micropapillary carcinoma, or mixed carcinoma.

The composition can be administered to a subject to prevent or to treat invasive breast cancer. The composition can be administered to the subject with invasive breast cancer to elicit an immune response, or to reduce or inhibit tumor growth in the subject.

The composition can also be administered to a subject to prevent or to treat ductal carcinoma in situ. The composition can be administered to the subject with ductal carcinoma in situ to elicit an immune response, or to reduce or inhibit tumor growth in the subject.

The composition can be formulated for subcutaneous, intramuscular, or intradermal administration.

The disclosure also provides for a kit for preparing the compositions described herein, the kit comprising instructions for preparing the composition. The disclosure also provides for a kit for administering the compositions described herein, the kit comprising instructions for administering the composition.

### Plasmids

A composition described herein may include a nucleic acid-based vaccine which comprises a plasmid encoding one or more epitopes selected from Otud6B, Pdhx, Stk39, Zfp238, Lgals8, or Vps35. Sometimes, a composition described herein may include a nucleic acid-based vaccine which comprises a plasmid encoding one or more epitopes selected from Otud6B, Pdhx, or Stk39. Sometimes, the epitopes may be derived from human proteins and the encoding nucleic acid sequences encoding the epitopes may be incorporated into a nucleic acid construct designed to induce expression of the epitope in a subject following administration. For example, epitopes encoded from the nucleic acid construct may allow for the immune response to at least one epitope to be entrained, amplified, attenuated, suppressed, or eliminated to specific sets of proteins (e.g., self-proteins).

The vaccine described herein can be a peptide based vaccine. The peptide based vaccine can comprise a plasmid which encodes one or more epitopes selected from Otud6B, Pdhx, Stk39, Zfp238, Lgals8, or Vps35. The peptide based vaccine can comprise a plasmid which encodes one or more epitopes selected from Otud6B, Pdhx, or Stk39. The epitopes may be derived from human proteins that may be used directly in a peptide based vaccine.

In some cases, the peptide or the nucleic acid construct may be optimized into a protein or plasmid-based vaccination to induce, amplify or entrain a T cell mediated immune response. Sometimes, the T cell mediated response is a Th1 immune response. The epitopes may be extended Th1 epitopes. Other times, the T cell mediated response is a Th2 immune response. The epitopes may be extended Th2 epitopes. In other cases, the peptide or the nucleic acid construct may be optimized into a protein or plasmid-based vaccination to suppress, attenuate or eliminate a pathological response, in a subject (e.g., human or animal) in need thereof.

The compositions described herein may include plasmids which contain nucleic acid sequences to express at least one epitope in a subject following administration of the composition (e.g., vaccine).

Any plasmid backbones (e.g., vectors) known to one of ordinary skill in the art suitable for pharmaceutical use for expression of a nucleic sequence may be used in the compositions described herein.

The vector can be a circular plasmid or a linear nucleic acid. The circular plasmid or linear nucleic acid can be capable of directing expression of a particular nucleotide sequence in an appropriate subject cell. The vector can have a promoter operably linked to the polypeptide-encoding nucleotide sequence, which can be operably linked to termination signals. The vector can also contain sequences required for proper translation of the nucleotide sequence. The vector comprising the nucleotide sequence of interest can be chimeric, meaning that at least one of its components is heterologous with respect to at least one of its other components. The expression of the nucleotide sequence in the expression cassette can be under the control of a constitutive promoter or of an inducible promoter, which can initiate transcription only when the host cell is exposed to some particular external stimulus.

The vector can be a plasmid. The plasmid can be useful for transfecting cells with nucleic acid encoding the polypeptide, which the transformed host cells can be cultured and maintained under conditions wherein expression of the polypeptide takes place.

The plasmid can comprise a nucleic acid sequence that encodes one or more of the various polypeptide disclosed herein. A single plasmid can contain coding sequence for a single polypeptide, or coding sequence for more than one polypeptide. Sometimes, the plasmid can further comprise coding sequence that encodes an adjuvant, such as an immune stimulating molecule, such as a cytokine.

The plasmid can further comprise an initiation codon, which can be upstream of the coding sequence, and a stop codon, which can be downstream of the coding sequence. The initiation and termination codon can be in frame with the coding sequence. The plasmid can also comprise a promoter that is operably linked to the coding sequence, and an enhancer upstream of the coding sequence. The enhancer can be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737.

The plasmid can also comprise a mammalian origin of replication in order to maintain the plasmid extrachromosomally and produce multiple copies of the plasmid in a cell. The plasmid can be pVAXI, pCEP4 or pREP4 from Invitrogen (San Diego, Calif.).

The plasmid can also comprise a regulatory sequence, which may be well suited for gene expression in a cell into which the plasmid is administered. The coding sequence can comprise a codon that can allow more efficient transcription of the coding sequence in the host cell.

In some cases, commercially available plasmid backbones may be used. For example, the plasmid pUMVC3 may be used. In some cases, commercially available plasmid backbones may be modified, mutated, engineered or cloned prior to use. In other cases, non-commercially available plasmid backbones may be used.

Additional plasmids can include pSE420 (Invitrogen, San Diego, Calif.), which can be used for protein production in Escherichia coli (E. coli). The plasmid can also be pYES2 (Invitrogen, San Diego, Calif.), which can be used for protein production in Saccharomyces cerevisiae strains of yeast. The plasmid can also be of the MAXBAC™ complete baculovirus expression system (Invitrogen, San Diego, Calif.), which can be used for protein production in insect cells. The plasmid can also be pcDNA I or pcDNA3 (Invitrogen, San Diego, Calif.), which can be used for protein production in mammalian cells such as Chinese hamster ovary (CHO) cells.

The vector can be circular plasmid, which can transform a target cell by integration into the cellular genome or exist extrachromosomally (e.g., autonomous replicating plasmid with an origin of replication). Exemplary vectors include pVAX, pcDNA3.0, or provax, or any other expression vector capable of expressing DNA encoding the antigen and enabling a cell to translate the sequence to an antigen that is recognized by the immune system.

The nucleic acid based vaccine can also be a linear nucleic acid vaccine, or linear expression cassette (“LEC”), that is capable of being efficiently delivered to a subject via electroporation and expressing one or more polypeptides disclosed herein. The LEC can be any linear DNA devoid of any phosphate backbone. The DNA can encode one or more polypeptides disclosed herein. The LEC can contain a promoter, an intron, a stop codon, and/or a polyadenylation signal. The expression of the polypeptide may be controlled by the promoter. The LEC cannot contain any antibiotic resistance genes and/or a phosphate backbone. The LEC cannot contain other nucleic acid sequences unrelated to the polypeptide expression.

The LEC can be derived from any plasmid capable of being linearized. The plasmid can express the polypeptide. Exemplary plasmids include: pNP (Puerto Rico/34), pM2 (New Caledonia/99), WLV009, pVAX, pcDNA3.0, provax, or any other expression vector capable of expressing DNA encoding the antigen and enabling a cell to translate the sequence to an antigen that is recognized by the immune system.

Prior to inserting the nucleic acid sequence of at least one epitope, the plasmid backbone may be less than about 500 bp, about 1.0 kB, about 1.2 kB, about 1.4 kB, about 1.6 kB, about 1.8 kB, about 2.0 kB, about 2.2 kB, about 2.4 kB, about 2.6 kB, about 2.8 kB, about 3.0 kB, about 3.2 kB, about 3.4 kB, about 3.6 kB, about 3.8 kB, about 4.0 kB, about 4.2 kB, about 4.4 kB, about 4.6 kB, about 4.8 kB, about 5.0 kB, about 5.2 kB, about 5.4 kB, about 5.6 kB, about 5.8 kB, about 6.0 kB, about 6.2 kB, about 6.4 kB, about 6.6 kB, about 6.8 kB, about 7.0 kB, about 7.2 kB, about 7.4 kB, about 7.6 kB, about 7.8 kB, about 8.0 kB, about 8.2 kB, about 8.4 kB, about 8.6 kB, about 8.8 kB, about 9.0 kB, about 9.2 kB, about 9.4 kB, about 9.6 kB, about 9.8 kB, about 10.0 kB, about 10.2 kB, about 10.4 kB, about 10.6 kB, about 10.8 kB, about 11.0 kB, about 11.2 kB, about 11.4 kB, about 11.6 kB, about 11.8 kB, about 12.0 kB, about 12.2 kB, about 12.4 kB, about 12.6 kB, about 12.8 kB, about 13.0 kB, about 13.2 kB, about 13.4 kB, about 13.6 kB, about 13.8 kB, about 14 kB, about 14.5 kB, about 15 kB, about 15.5 kB, about 16 kB, about 16.5 kB, about 17 kB, about 17.5 kB, about 18 kB, about 18.5 kB, about 19 kB, about 19.5 kB, about 20 kB, about 30 kB, about 40 kB, about 50 kB, about 60 kB, about 70 kB, about 80 kB, about 90 kB, about 100 kB, about 110 kB, about 120 kB, about 130 kB, about 140 kB, about 150 kB, about 160 kB, about 170 kB, about 180 kB, about 190 kB or about 200 kB in length. In an exemplary case, the plasmid is about 4 kB in length prior to addition of the nucleic acid sequence encoding at least one epitope.

In some cases, the compositions described herein may include one plasmid. In other cases, the compositions described herein may include more than one plasmid. For example, the compositions described herein may include two plasmids, three plasmids, four plasmids, five plasmids, six plasmids, seven plasmids, eight plasmids, nine plasmids, ten plasmids, 11 plasmids, 12 plasmids, 13 plasmids, 14 plasmids, 15 plasmids, 16 plasmids, 17 plasmids, 18 plasmids 19 plasmids, 20 plasmids or more than 20 plasmids.

The nucleic acids which encode at least one epitope of a plasmid may be derived from any species such that the epitope expressed from the nucleic acids results in an immune response in a subject. In some cases, the subject may be a rodent, a non-human primate or a human. The nucleic acids encoding the epitope of the plasmid may be isolated from any source of nucleic acids using methods and techniques known to one of ordinary skill in the art. The nucleic acids encoding the epitope of the plasmid may be cloned into the plasmid backbone using methods and techniques known to one of ordinary skill in the art.

In some cases, the nucleic acid sequence encoding the epitope may be an endogenous nucleic acid sequence to the subject. For example, the nucleic acid sequence for Otud6B from a human may be used to express Otud6B in a human. In other cases, the nucleic acid sequence for the antigenic epitope may be an exogenous nucleic acid sequence to the subject. For example, the nucleic acid sequence for Otud6B from a non-human may be used to express Otud6B in a human.

In some cases, the nucleic acid sequences to express the antigenic epitope may be wild-type nucleic acid sequences. For example, the naturally occurring nucleic acid sequence for Otud6B in the genome of a species may be used to express Otud6B in a subject. In other cases, the nucleic acid sequences encoding the epitope may be synthetic nucleic acid sequences. For example, the naturally occurring nucleic acid sequence for Otud6B in the genome of a species may be modified using molecular techniques known to one of ordinary skill in the art and may be used to express Otud6B in a subject.

In some cases, the nucleic acid sequence encoding the epitope may be an endogenous nucleic acid sequence to the subject. For example, the nucleic acid sequence for Pdhx from a human may be used to express Pdhx in a human. In other cases, the nucleic acid sequence for the antigenic epitope may be an exogenous nucleic acid sequence to the subject. For example, the nucleic acid sequence for Pdhx from a non-human may be used to express Pdhx in a human.

In some cases, the nucleic acid sequences to express the antigenic epitope may be wild-type nucleic acid sequences. For example, the naturally occurring nucleic acid sequence for Pdhx in the genome of a species may be used to express Pdhx in a subject. In other cases, the nucleic acid sequences encoding the epitope may be synthetic nucleic acid sequences. For example, the naturally occurring nucleic acid sequence for Pdhx in the genome of a species may be modified using molecular techniques known to one of ordinary skill in the art and may be used to express Pdhx in a subject.

In some cases, the nucleic acid sequence encoding the epitope may be an endogenous nucleic acid sequence to the subject. For example, the nucleic acid sequence for Stk39 from a human may be used to express Stk39 in a human. In other cases, the nucleic acid sequence for the antigenic epitope may be an exogenous nucleic acid sequence to the subject. For example, the nucleic acid sequence for Stk39 from a non-human may be used to express Stk39 in a human.

In some cases, the nucleic acid sequences to express the antigenic epitope may be wild-type nucleic acid sequences. For example, the naturally occurring nucleic acid sequence for Stk39 in the genome of a species may be used to express Stk39 in a subject. In other cases, the nucleic acid sequences encoding the epitope may be synthetic nucleic acid sequences. For example, the naturally occurring nucleic acid sequence for Stk39 in the genome of a species may be modified using molecular techniques known to one of ordinary skill in the art and may be used to express Stk39 in a subject.

In some cases, the nucleic acid sequence encoding the epitope may be an endogenous nucleic acid sequence to the subject. For example, the nucleic acid sequence for Zfp238 from a human may be used to express Zfp238 in a human. In other cases, the nucleic acid sequence for the antigenic epitope may be an exogenous nucleic acid sequence to the subject. For example, the nucleic acid sequence for Zfp238 from a non-human may be used to express Zfp238 in a human.

In some cases, the nucleic acid sequences to express the antigenic epitope may be wild-type nucleic acid sequences. For example, the naturally occurring nucleic acid sequence for Zfp238 in the genome of a species may be used to express Zfp238 in a subject. In other cases, the nucleic acid sequences encoding the epitope may be synthetic nucleic acid sequences. For example, the naturally occurring nucleic acid sequence for Zfp238 in the genome of a species may be modified using molecular techniques known to one of ordinary skill in the art and may be used to express Zfp238 in a subject.

In some cases, the nucleic acid sequence encoding the epitope may be an endogenous nucleic acid sequence to the subject. For example, the nucleic acid sequence for Lgals8 from a human may be used to express Lgals8 in a human. In other cases, the nucleic acid sequence for the antigenic epitope may be an exogenous nucleic acid sequence to the subject. For example, the nucleic acid sequence for Lgals8 from a non-human may be used to express Lgals8 in a human.

In some cases, the nucleic acid sequences to express the antigenic epitope may be wild-type nucleic acid sequences. For example, the naturally occurring nucleic acid sequence for Lgals8 in the genome of a species may be used to express Lgals8 in a subject. In other cases, the nucleic acid sequences encoding the epitope may be synthetic nucleic acid sequences. For example, the naturally occurring nucleic acid sequence for Lgals8 in the genome of a species may be modified using molecular techniques known to one of ordinary skill in the art and may be used to express Lgals8 in a subject.

In some cases, the nucleic acid sequence encoding the epitope may be an endogenous nucleic acid sequence to the subject. For example, the nucleic acid sequence for Vps35 from a human may be used to express Vps35 in a human. In other cases, the nucleic acid sequence for the antigenic epitope may be an exogenous nucleic acid sequence to the subject. For example, the nucleic acid sequence for Vps35 from a non-human may be used to express Vps35 in a human.

In some cases, the nucleic acid sequences to express the antigenic epitope may be wild-type nucleic acid sequences. For example, the naturally occurring nucleic acid sequence for Vps35 in the genome of a species may be used to express Vps35 in a subject. In other cases, the nucleic acid sequences encoding the epitope may be synthetic nucleic acid sequences. For example, the naturally occurring nucleic acid sequence for Vps35 in the genome of a species may be modified using molecular techniques known to one of ordinary skill in the art and may be used to express Vps35 in a subject.

Sometimes, plasmids comprising more than one epitope sequences may comprise spacers between each epitope sequence. In some cases, sequences of the epitopes may be encoded in tandem without the use of spacers. In some cases, sequences of epitopes may be encoded in tandem with the use of spacers. In some cases, the spacers may comprise sequences encoding from about 1 to about 50, about 3 to about 40, about 5 to about 35, or about 10 to about 30 amino acid residues. In some instances, the spacers may comprise sequences encoding about 1, 2, 3, 4, 5, 6, 7, 8, 9, 10, 11, 12, 13, 14, 15, 16, 17, 18, 19, 20, 25, or 30 amino acid residues.

In some cases, the plasmid may contain a nucleic acid sequence coding for at least one tag. In some cases, the tag may be translated into a peptide. Any nucleic acid sequence for a tag known to one of ordinary skill in the art may be used with the plasmids described herein. For example, the tag may be a histidine tag with three histidine residues, a histidine tag with four histidine residues, a histidine tag with five histidine residues, or a histidine tag with six histidine residues, or the like. Expression of the tag in a subject may be determined using any suitable technique known to one of ordinary skill in the art.

In some cases, plasmids may be sequenced using any sequencing technique known to one of ordinary skill in the art such that the results of the sequencing technique provides nucleotide level resolution of the entire plasmid.

In some aspects, the composition may be a multiantigen breast cancer vaccine. For example, the multiantigen breast cancer vaccine may contain a plurality of antigens. In some cases, expression of one antigen may impact expression of a different antigen. In some cases, expression of more than one antigen may impact expression of a different antigen. In some cases, expression of one antigen may impact expression of more than one different antigen. In some cases, expression of one antigen may not impact expression of a different antigen. In some cases, expression of more than one antigen may not impact expression of a different antigen. In some cases, expression of one antigen may not impact expression of more than one different antigen. For example, antigenic competition may limit the immunogenicity of multiantigen vaccines. Any techniques known to one of ordinary skill in the art may be used to determine if an immune response elicited following administration of a multiple antigen vaccine is of comparable magnitude to each antigen as a single antigen vaccine. For example, ELISPOT (e.g., for secretion of IFNγ) may determine the magnitude of the immune response. In some cases, the ELISPOT may detect rodent, non-human primate or human peptides. In some instances, the multiantigen breast cancer vaccine may comprise a plurality of epitopes derived from a plurality of antigens selected from Otud6B, Pdhx, Stk39, Zfp238, Lgals8, and/or Vps35.

### Nucleic Acids

An isolated nucleic acid molecule is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified, but indicates that the molecule does not include an entire genome or an entire chromosome in which the nucleic acid molecule is found in nature. An isolated nucleic acid molecule can include a gene. An isolated nucleic acid molecule that includes a gene is not a fragment of a chromosome that includes such gene, but rather includes the coding region and regulatory regions associated with the gene, but no additional genes that are naturally found on the same chromosome. An isolated nucleic acid molecule can also include a specified nucleic acid sequence flanked by (i.e., at the 5′ and/or the 3′ end of the sequence) additional nucleic acids that do not normally flank the specified nucleic acid sequence in nature (i.e., heterologous sequences).

Isolated nucleic acid molecule can include DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein or domain of a protein.

Nucleic acid molecules may refer to at least two nucleotides covalently linked together. A nucleic acid described herein can contain phosphodiester bonds, although in some cases, as outlined below (for example in the construction of primers and probes such as label probes), nucleic acid analogs are included that can have alternate backbones, comprising, for example, phosphoramide (Beaucage et al., Tetrahedron 49(10): 1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 (1986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989)), O-methylphosphoroamidite linkages (see Eckstein, Oligonucleotides and Analogues: A Practical Approach, Oxford University Press), and peptide nucleic acid (also referred to herein as “PNA”) backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996)), all of which are incorporated by reference. Other analog nucleic acids include those with bicyclic structures including, locked nucleic acids (also referred to herein as “LNA”) (Koshkin et al., J. Am. Chem. Soc. 120.13252 3 (1998)); positive backbones (Denpcy et al., Proc. Natl. Acad. Sci. USA 92:6097 (1995)); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)); and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169-176). Several nucleic acid analogs are described in Rawls, C & E News Jun. 2, 1997 page 35. “Locked nucleic acids” are also included within the definition of nucleic acid analogs. LNAs are a class of nucleic acid analogues in which the ribose ring is “locked” by a methylene bridge connecting the 2′-O atom with the 4′-C atom. All of these references are hereby expressly incorporated by reference. These modifications of the ribose-phosphate backbone can be done to increase the stability and half-life of such molecules in physiological environments. For example, PNA:DNA and LNA-DNA hybrids can exhibit higher stability and thus can be used in some embodiments. The target nucleic acids can be single stranded or double stranded, as specified, or contain portions of both double stranded or single stranded sequence. Depending on the application, the nucleic acids can be DNA (including, e.g., genomic DNA, mitochondrial DNA, and cDNA), RNA (including, e.g., mRNA and rRNA) or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

A recombinant nucleic acid molecule is a molecule that can include at least one of any nucleic acid sequence encoding any one or more proteins described herein operatively linked to at least one of any transcription control sequence capable of effectively regulating expression of the nucleic acid molecule(s) in the cell to be transfected. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein. In addition, the phrase “recombinant molecule” primarily refers to a nucleic acid molecule operatively linked to a transcription control sequence, but can be used interchangeably with the phrase “nucleic acid molecule” which is administered to an animal.

A recombinant nucleic acid molecule includes a recombinant vector, which is any nucleic acid sequence, typically a heterologous sequence, which is operatively linked to the isolated nucleic acid molecule encoding a fusion protein of the present invention, which is capable of enabling recombinant production of the fusion protein, and which is capable of delivering the nucleic acid molecule into a host cell according to the present invention. Such a vector can contain nucleic acid sequences that are not naturally found adjacent to the isolated nucleic acid molecules to be inserted into the vector. The vector can be either RNA or DNA, either prokaryotic or eukaryotic, and preferably in the present invention, is a virus or a plasmid. Recombinant vectors can be used in the cloning, sequencing, and/or otherwise manipulating of nucleic acid molecules, and can be used in delivery of such molecules (e.g., as in a DNA composition or a viral vector-based composition). Recombinant vectors are preferably used in the expression of nucleic acid molecules, and can also be referred to as expression vectors. Preferred recombinant vectors are capable of being expressed in a transfected host cell.

In a recombinant molecule of the present invention, nucleic acid molecules are operatively linked to expression vectors containing regulatory sequences such as transcription control sequences, translation control sequences, origins of replication, and other regulatory sequences that are compatible with the host cell and that control the expression of nucleic acid molecules of the present invention. In particular, recombinant molecules of the present invention include nucleic acid molecules that are operatively linked to one or more expression control sequences. The phrase “operatively linked” refers to linking a nucleic acid molecule to an expression control sequence in a manner such that the molecule is expressed when transfected (i.e., transformed, transduced or transfected) into a host cell.

Compositions Comprising Nucleic Acid Polymers that Hybridize to an Antigen Target Sequence for a Breast Cancer Vaccine

The compositions described herein further include a nucleic acid polymer that hybridizes to a target sequence encoding a pre-diagnostic antigen described herein. In some instances, the compositions described herein include a nucleic acid polymer that hybridizes to a target sequence encoding Otud6B, Pdhx, Stk39, Zfp238, Lgals8, or Vps35, wherein the nucleic acid polymer modulates gene expression of the target sequence. Sometimes, the compositions can include a nucleic acid polymer that hybridizes to a target sequence encoding Otud6B, Pdhx, or Stk39, wherein the nucleic acid polymer modulates gene expression of the target sequence. The composition can comprise a nucleic acid polymer that hybridizes to a target sequence encoding Zfp238, Lgals8, or Vps35, wherein the nucleic acid polymer modulates the gene expression of the target sequence. The compositions can further include a plurality of nucleic acid polymers that hybridize to a group of target sequences in which each target sequence from the group encodes a gene selected from Otud6B, Pdhx, Stk39, Zfp238, Lgals8, or Vps35, wherein the plurality of nucleic acid polymers modulates the gene expression of the group of target sequences.

The nucleic acid polymer can modulate gene expression of the target sequence. As used herein, gene expression encompasses the gene product of a target sequence. The gene product can include functional proteins or functional RNAs. In some instances, the nucleic acid polymer can decrease gene expression (e.g., gene product) of the target sequence. For example, the nucleic acid polymer can lead to an increase in the population of non-functional or aberrant version of proteins or RNAs and a decrease to the production of functional copies. Sometimes, a decrease in the gene expression of the target sequence can lead to an increase in apoptosis of tumor cells.

The nucleic acid polymer may be about 50 nucleotides in length. The nucleic acid polymer may be about 45, 40, 35, 30, 25, 24, 23, 22, 21, 20, 19, 18, 17, 16, 15, 14, 13, 12, 11, 10, 9, 8, 7, 6, or 5 nucleotides in length. Sometimes, the nucleic acid polymer may be between about 10 and about 50 nucleotides in length. The nucleic acid polymer may be between about 10 and about 45 nucleotides in length. The nucleic acid polymer may be between about 10 and about 40 nucleotides in length. The nucleic acid polymer may be between about 10 and about 35 nucleotides in length. The nucleic acid polymer may be between about 10 and about 20 nucleotides in length. The nucleic acid polymer may be between about 10 and about 25 nucleotides in length. The nucleic acid polymer may be between about 10 and about 20 nucleotides in length. The nucleic acid polymer may be between about 15 and about 50 nucleotides in length. The nucleic acid polymer may be between about 15 and about 40 nucleotides in length. The nucleic acid polymer may be between about 15 and about 35 nucleotides in length. The nucleic acid polymer may be between about 15 and about 30 nucleotides in length. The nucleic acid polymer may be between about 20 and about 45 nucleotides in length. The nucleic acid polymer may be between about 20 and about 40 nucleotides in length. The nucleic acid polymer may be between about 20 and about 30 nucleotides in length.

The nucleic acid polymer may comprise RNA, DNA, or a combination thereof. The nucleic acid polymer may comprise natural or artificial (or synthetic) nucleotide analogues, in which the artificial nucleotide analogues have equivalent complementation as DNA or RNA. The artificial nucleotide analogues may include modifications at one or more of ribose moiety, phosphate moiety, nucleoside moiety, or a combination thereof.

Artificial nucleotide analogues may comprise a nucleic acid with a modification at a 2′ hydroxyl group of the ribose moiety. The modification can be a 2′-O-methyl modification or a 2′-O-methoxyethyl (2′-O-MOE) modification. The 2′-O-methyl modification can add a methyl group to the 2′ hydroxyl group of the ribose moiety whereas the 2′O-methoxyethyl modification can add a methoxyethyl group to the 2′ hydroxyl group of the ribose moiety. Exemplary chemical structures of a 2′-O-methyl modification of an adenosine molecule and 2′O-methoxyethyl modification of a uridine are illustrated below.

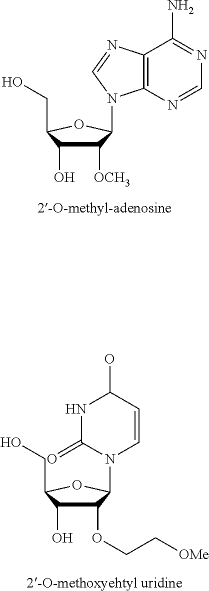

An additional modification at the 2′ hydroxyl group can include a 2′-O-aminopropyl sugar conformation which can involve an extended amine group comprising a propyl linker that binds the amine group to the 2′ oxygen. This modification can neutralize the phosphate derived overall negative charge of the oligonucleotide molecule by introducing one positive charge from the amine group per sugar and can thereby improve cellular uptake properties due to its zwitterionic properties. An exemplary chemical structure of a 2′-O-aminopropyl nucleoside phosphoramidite is illustrated below.

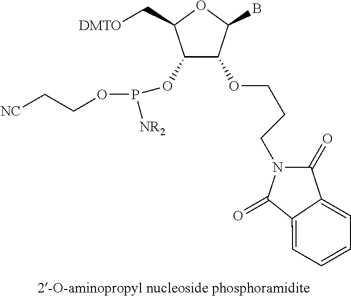

Another modification at the 2′ hydroxyl group can include a locked or bridged ribose conformation (e.g., locked nucleic acid or LNA) where the 4′ ribose position can also be involved. In this modification, the oxygen molecule bound at the 2′ carbon can be linked to the 4′ carbon by a methylene group, thus forming a 2′-C,4′-C-oxy-methylene-linked bicyclic ribonucleotide monomer. Exemplary representations of the chemical structure of LNA are illustrated below. The representation shown to the left highlights the chemical connectivities of an LNA monomer. The representation shown to the right highlights the locked 3′-endo (³E) conformation of the furanose ring of an LNA monomer.

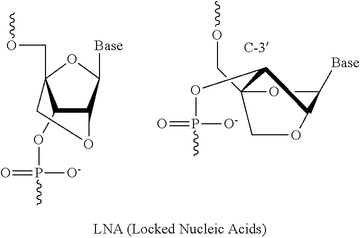

A further modification at the 2′ hydroxyl group may comprise ethylene nucleic acids (ENA) such as for example 2′-4′-ethylene-bridged nucleic acid, which locks the sugar conformation into a C₃′-endo sugar puckering conformation. ENA are part of the bridged nucleic acids class of modified nucleic acids that also comprises LNA. Exemplary chemical structures of the ENA and bridged nucleic acids are illustrated below.

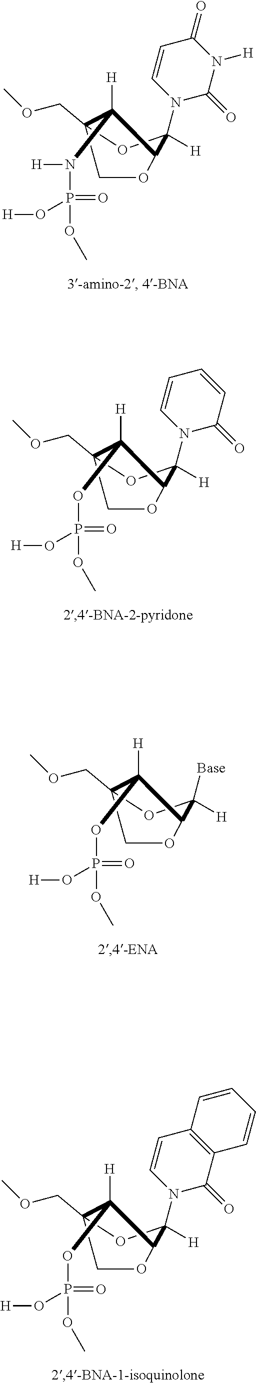

Still other modifications at the 2′ hydroxyl group can include 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O—NMA).

Nucleotide analogues may further comprise Morpholinos, peptide nucleic acids (PNAs), methylphosphonate nucleotides, thiolphosphonate nucleotides, 2′-fluoro N3-P5′-phosphoramidites, 1′, 5′-anhydrohexitol nucleic acids (HNAs), or a combination thereof. Morpholino or phosphorodiamidate morpholino oligo (PMO) comprises synthetic molecules whose structure mimics natural nucleic acid structure by deviates from the normal sugar and phosphate structures. Instead, the five member ribose ring can be substituted with a six member morpholino ring containing four carbons, one nitrogen and one oxygen. The ribose monomers can be linked by a phosphordiamidate group instead of a phosphate group. These backbone alterations can remove all positive and negative charges making morpholinos neutral molecules that can cross cellular membranes without the aid of cellular delivery agents such as those used by charged oligonucleotides.

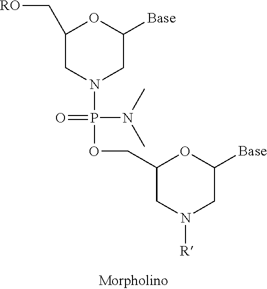

Peptide nucleic acid (PNA) does not contain sugar ring or phosphate linkage. Instead, the bases can be attached and appropriately spaced by oligoglycine-like molecules, therefore, eliminating a backbone charge.

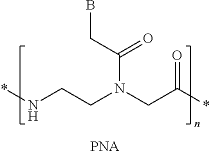

Modification of the phosphate backbone may also comprise methyl or thiol modifications such as methylphosphonate nucleotide and. Exemplary thiolphosphonate nucleotide (left) and methylphosphonate nucleotide (right) are illustrated below.

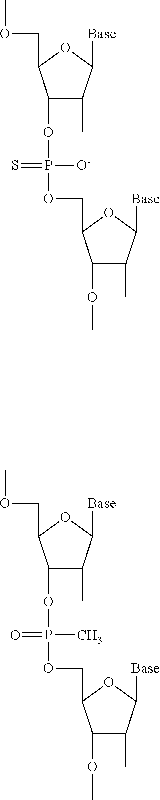

Furthermore, exemplary 2′-fluoro N3-P5′-phosphoramidites is illustrated as:

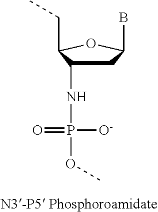

And exemplary hexitol nucleic acid (or 1′, 5′-anhydrohexitol nucleic acids (HNA)) is illustrated as:

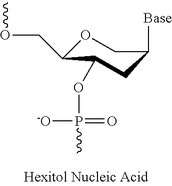

In addition to modification of the ribose moiety, phosphate backbone and the nucleoside, the nucleotide analogues can also be modified by for example at the 3′ or the 5′ terminus. For example, the 3′ terminus can include a 3′ cationic group, or by inverting the nucleoside at the 3′-terminus with a 3′-3′ linkage. In another alternative, the 3′-terminus can be blocked with an aminoalkyl group, e.g., a 3′ C5-aminoalkyl dT. The 5′-terminus can be blocked with an aminoalkyl group, e.g., a 5′-O-alkylamino substituent. Other 5′ conjugates can inhibit 5′-3′ exonucleolytic cleavage. Other 3′ conjugates can inhibit 3′-5′ exonucleolytic cleavage.

In some cases, one or more of the artificial nucleotide analogues described herein are resistant toward nucleases such as for example ribonuclease such as RNase H, deoxyribunuclease such as DNase, or exonuclease such as 5′-3′ exonuclease and 3′-5′ exonuclease when compared to natural polynucleic acid polymers. Artificial nucleotide analogues comprising 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O—NMA) modified, LNA, ENA, PNA, HNA, morpholino, methylphosphonate nucleotides, thiolphosphonate nucleotides, 2′-fluoro N3-P5′-phosphoramidites, or combinations thereof can be resistant toward nucleases such as for example ribonuclease such as RNase H, deoxyribunuclease such as DNase, or exonuclease such as 5′-3′ exonuclease and 3′-5′ exonuclease, 2′-O-methyl modified polynucleic acid polymer may be nuclease resistance (e.g., RNase H, DNase, 5′-3′ exonuclease or 3′-5′ exonuclease resistance), 2′O-methoxyethyl (2′-O-MOE) modified polynucleic acid polymer may be nuclease resistance (e.g., RNase H, DNase, 5′-3′ exonuclease or 3′-5′ exonuclease resistance), 2′-O-aminopropyl modified polynucleic acid polymer may be nuclease resistance (e.g., RNase H, DNase, 5′-3′ exonuclease or 3′-5′ exonuclease resistance), 2′-deoxy modified polynucleic acid polymer may be nuclease resistance (e.g., RNase H, DNase, 5′-3′ exonuclease or 3′-5′ exonuclease resistance). T-deoxy-2′-fluoro modified polynucleic acid polymer may be nuclease resistance (e.g., RNase H, DNase, 5′-3′ exonuclease or 3′-5′ exonuclease resistance), 2′-O-aminopropyl (2′-O-AP) modified polynucleic acid polymer may be nuclease resistance (e.g., RNase H, DNase, 5′-3′ exonuclease or 3′-5′ exonuclease resistance), 2′-O-dimethylaminoethyl (2′-O-DMAOE) modified polynucleic acid polymer may be nuclease resistance (e.g., RNase H, DNase, 5′-3′ exonuclease or 3′-5′ exonuclease resistance), 2′-O-dimethylaminopropyl (2′-O-DMAP) modified polynucleic acid polymer may be nuclease resistance (e.g., RNase H, DNase, 5′-3′ exonuclease or 3′-5′ exonuclease resistance). T-O-dimethylaminoethyloxyethyl (2′-O-DMAEOE) modified polynucleic acid polymer may be nuclease resistance (e.g., RNase H, DNase, 5′-3′ exonuclease or 3′-5′ exonuclease resistance), 2′-O—N-methylacetamido (2′-O—NMA) modified polynucleic acid polymer may be nuclease resistance (e.g., RNase H, DNase, 5′-3′ exonuclease or 3′-5′ exonuclease resistance). LNA modified polynucleic acid polymer may be nuclease resistance (e.g., RNase H, DNase, 5′-3′ exonuclease or 3′-5′ exonuclease resistance). ENA modified polynucleic acid polymer may be nuclease resistance (e.g., RNase H, DNase, 5′-3′ exonuclease or 3′-5′ exonuclease resistance). HNA modified polynucleic acid polymer may be nuclease resistance (e.g., RNase H, DNase, 5′-3′ exonuclease or 3′-5′ exonuclease resistance). Morpholinos may be nuclease resistance (e.g., RNase H, DNase, 5′-3′ exonuclease or 3′-5′ exonuclease resistance). PNA can be resistant to nucleases (e.g., RNase H, DNase, 5′-3′ exonuclease or 3′-5′ exonuclease resistance). Methylphosphonate nucleotides modified polynucleic acid polymer may be nuclease resistance (e.g., RNase H, DNase, 5′-3′ exonuclease or 3′-5′ exonuclease resistance). Thiolphosphonate nucleotides modified polynucleic acid polymer may be nuclease resistance (e.g., RNase H, DNase, 5′-3′ exonuclease or 3′-5′ exonuclease resistance). Polynucleic acid polymer comprising 2′-fluoro N3-P5′-phosphoramidites may be nuclease resistance (e.g., RNase H, DNase, 5′-3′ exonuclease or 3′-5′ exonuclease resistance).

One or more of the artificial nucleotide analogues described herein may also be modified to increase its stability. Artificial nucleotide analogues comprising 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O—NMA) modified, LNA, ENA, PNA, HNA, morpholino, methylphosphonate nucleotides, thiolphosphonate nucleotides, 2′-fluoro N3-P5′-phosphoramidites, or combinations thereof can be modified to increase its stability.

In some instances, the nucleic acid polymers have greater than 80% sequence identity to the target sequence. Sometimes, the nucleic acid polymer have greater than 85%, 90%, 91%, 92%, 93%, 94%, 95%, 96%, 97%, 98%, or 99% sequence identity to the target sequence.

In some cases, the nucleic acid polymer is an RNA polymer. The nucleic acid polymer can be an antisense polymer. The nucleic acid polymer can be a microRNA, a microRNA precursor, small interfering RNA (siRNA), or short hairpin RNA (shRNA). The nucleic acid polymer can be chemically synthesized or expressed in a cell or cell-free system.

Methods of synthesizing a nucleic acid polymer (e.g., RNA polymers) are known in the art, in particular, the chemical synthesis methods as described in Verma and Eckstein (1998) Annul Rev. Biochem. 67:99-134. For example, a nucleic acid polymer such as a ds-siRNA can be prepared by enzymatic processing of a long ds RNA having sufficient complementarity to the desired target mRNA. Processing of long ds RNA can be accomplished in vitro, for example, using appropriate cellular lysates and ds-siRNAs can be subsequently purified by gel electrophoresis or gel filtration. ds-siRNA can then be denatured according to art-recognized methodologies. In an example, RNA polymer can be purified from a mixture by extraction with a solvent or resin, precipitation, electrophoresis, chromatography, or a combination thereof. Alternatively, the RNA polymer may be used with no or a minimum of purification to avoid losses due to sample processing. Alternatively, the single-stranded RNA polymers can also be prepared by enzymatic transcription from synthetic DNA templates or from DNA plasmids isolated from recombinant bacteria. Typically, phage RNA polymerases are used such as T7, T3 or SP6 RNA polymerase (Milligan and Uhlenbeck (1989) Methods Enzymol. 180:51-62). The RNA polymer may be dried for storage or dissolved in an aqueous solution. The solution may contain buffers or salts to inhibit annealing, and/or promote stabilization of the single strands.

### Methods of Assessing an Individual's Risk for Developing Breast Cancer

Disclosed herein include methods for identifying an individual's risk for developing breast cancer. The individual can be at risk for developing ductal carcinoma in situ, lobular carcinoma in situ, invasive ductal carcinoma, infiltrating ductal carcinoma, inflammatory breast cancer, triple-negative breast cancer, paget disease of the nipple, phyllodes tumor, angiosarcoma, adenoid cystic carcinoma, adenocystic carcinoma, low-grade adenosquamous carcinoma, medullary carcinoma, mucinous carcinoma, colloid carcinoma, papillary carcinoma, tubular carcinoma, metaplastic carcinoma, spindle cell carcinoma, squamous carcinoma, micropapillary carcinoma, or mixed carcinoma. The individual can be at risk for developing invasive breast cancer. The individual can be at risk for developing ductal carcinoma in situ.

The method can comprise incubating a biological sample from a subject with a probe to detect the presence and concentration level of an autoantibody selected from Otud6B, Pdhx, Stk39, or a combination thereof; and based on the presence or the concentration level of the autoantibody, assessing whether the subject is at risk for developing breast cancer.

The method can also comprise incubating a biological sample from a subject with a panel of probes, in which each probe from the panel can detect the presence and concentration level of an autoantibody selected from Otud6B, Pdhx, Stk39, or a combination thereof; and based on the presence or the concentration level of the autoantibody, assessing whether the subject is at risk for developing breast cancer.

Sometimes, the method further includes detecting the presence and concentration levels of autoantibodies Zfp238, Lgals8, or Vps35, or a combination thereof.

In some cases, the method comprises the steps of a) incubating a biological sample from the subject with at least a first probe, wherein the biological sample comprises an autoantibody selected from Otud6B, Pdhx, Stk39, or a combination thereof and the first probe comprises a recombinant polypeptide comprising an antigen of Otud6B, Pdhx, Stk39, or a combination thereof; b) forming a first autoantibody-probe complex comprising the autoantibody and the first probe of step a); c) measuring the concentration of the first autoantibody-probe complex, thereby determining the concentration of the autoantibody; and d) identifying the subject as at risk for developing breast cancer if the subject has an elevated concentration of the autoantibody relative to a control.

The method can further comprise i) incubating the biological sample with at least a third probe, wherein the third probe comprises a second recombinant polypeptide comprising an antigen of Zfp238, Lgals8, or Vps35, or a combination thereof; ii) forming a second autoantibody-probe complex comprising the autoantibody and the third probe of step i); and iii) measuring the concentration of the second autoantibody-probe complex, thereby determining the concentration of an autoantibody of Zfp238, Lgals8, or Vps35, or a combination thereof.

In some instances, the method comprises the steps of a) incubating a biological sample from the subject with a panel of probes, wherein the biological sample comprises autoantibodies selected from Otud6B, Pdhx, Stk39, Zfp238, Lgals8, Vps35, or a combination thereof and the panel of probes comprise recombinant polypeptides in which each recombinant polypeptide comprises an antigen of Otud6B, Pdhx, Stk39, Zfp238, Lgals8, Vps35, or a combination thereof; b) forming an autoantibody-probe complex comprising an autoantibody and a probe from the panel of probes; c) measuring the concentration of the autoantibody-probe complex, thereby determining the concentration of the autoantibody; and d) identifying the subject as at risk for developing breast cancer if the subject has an elevated concentration of the autoantibody relative to a control.

The autoantibody can be an IgG or an IgM autoantibody. Alternatively, the autoantibody can be an IgA, an IgD, or an IgE autoantibody.

Sometimes, the method includes detecting the presence and concentration level of IgG, IgM, IgA, IgD, IgE autoantibodies or a combination thereof, to pre-diagnostic antigen Otud6B, Pdhx, Stk39, Zfp238, Lgals8, Vps35, or combinations thereof. The method can include detecting the presence and concentration level of IgG autoantibody to Otud6B, Pdhx, Stk39, Zfp238, Lgals8, Vps35, or combinations thereof. The method can include detecting the presence and concentration level of IgM autoantibody to Otud6B, Pdhx, Stk39, Zfp238, Lgals8, Vps35, or combinations thereof. The method can include detecting the presence and concentration level of IgA autoantibody to Otud6B, Pdhx, Stk39, Zfp238, Lgals8, Vps35, or combinations thereof. The method can include detecting the presence and concentration level of IgD autoantibody to Otud6B, Pdhx, Stk39, Zfp238, Lgals8, Vps35, or combinations thereof. The method can include detecting the presence and concentration level of IgE autoantibody to Otud6B, Pdhx, Stk39, Zfp238, Lgals8, Vps35, or combinations thereof.

Sometimes, the method includes detecting the presence and concentration levels of at least two types of autoantibodies (e.g., IgG and IgM, IgG and IgA, IgM and IgA) to Otud6B, Pdhx, Stk39, Zfp238, Lgals8, Vps35, or combinations thereof. Sometimes, the method includes detecting the presence and concentration levels of at least three types of autoantibodies (e.g., IgG, IgM, and IgA) to Otud6B, Pdhx, Stk39, Zfp238, Lgals8, Vps35, or combinations thereof. Sometimes, the method includes detecting the presence and concentration levels of both IgG and IgM autoantibodies to Otud6B, Pdhx, Stk39, or combinations thereof.

The control can be a biological sample obtained from a subject who is not at risk for developing breast cancer. The control can also be a biological sample obtained from a subject who does not have breast cancer.

The recombinant polypeptide can be a polypeptide comprising at least 70%, 80%, 85%, 90%, 95%, 99% or 100% sequence identity to Otud6B. The first recombinant polypeptide can be a polypeptide comprising at least 70%, 80%, 85%, 90%, 95%, 99% or 100% sequence identity to SEQ ID NO: 1 (Otud6B).

The recombinant polypeptide can be a polypeptide comprising at least 70%, 80%, 85%, 90%, 95%, 99% or 100% sequence identity to Pdhx. The first recombinant polypeptide can be a polypeptide comprising at least 70%, 80%, 85%, 90%, 95%, 99% or 100% sequence identity to SEQ ID NO: 3 (Pdhx).

The recombinant polypeptide can be a polypeptide comprising at least 70%, 80%, 85%, 90%, 95%, 99% or 100% sequence identity to Stk39. The first recombinant polypeptide can be a polypeptide comprising at least 70%, 80%, 85%, 90%, 95%, 99% or 100% sequence identity to SEQ ID NO: 5 (Stk39).

The second recombinant polypeptide can be a polypeptide comprising at least 70%, 80%, 85%, 90%, 95%, 99% or 100% sequence identity to Zfp238. The second recombinant polypeptide can be a polypeptide comprising at least 70%, 80%, 85%, 90%, 95%, 99% or 100% sequence identity to SEQ ID NO: 8 (Zfp238).

The second recombinant polypeptide can be a polypeptide comprising at least 70%, 80%, 85%, 90%, 95%, 99% or 100% sequence identity to Lgals8. The second recombinant polypeptide can be a polypeptide comprising at least 70%, 80%, 85%, 90%, 95%, 99% or 100% sequence identity to SEQ ID NO: 10 (Lgals8).

The second recombinant polypeptide can be a polypeptide comprising at least 70%, 80%, 85%, 90%, 95%, 99% or 100% sequence identity to Vps35. The second recombinant polypeptide can be a polypeptide comprising at least 70%, 80%, 85%, 90%, 95%, 99% or 100% sequence identity to SEQ ID NO: 12 (Vps35).

The method can further comprise incubating the biological sample with at least a second probe, wherein the second probe is a secondary antibody; forming an autoantibody-probe-second probe complex; and measuring the concentration of the autoantibody-probe-second probe complex, thereby determining the concentration of the autoantibody.

The biological sample can be a serum sample.

The method can further comprise administering to the subject a composition comprising an isolated and purified plasmid comprising at least one nucleotide sequence encoding a polypeptide comprising at least 70% sequence identity to an epitope sequence of SEQ ID NO: 1 (Otud6B), SEQ ID NO: 3 (Pdhx), or SEQ ID NO: 5 (Stk39); or a nucleic acid polymer that hybridizes to a target sequence encoding Otud6B, Pdhx, or Stk39, wherein the nucleic acid polymer modulates the gene expression of the target sequence; thereby reducing the risk of or preventing breast cancer in the subject.

As described above, the isolated and purified plasmid can comprise at least one nucleotide sequence encoding a polypeptide comprising at least 80%, 85%, 90%, 95%, 99%, or 100% sequence identity to an epitope sequence of SEQ ID NO: 1. The isolated and purified plasmid can comprise at least one nucleotide sequence encoding a polypeptide comprising at least 80%, 85%, 90%, 95%, 99%, or 100% sequence identity to an epitope sequence of SEQ ID NO: 3. The isolated and purified plasmid can comprise at least one nucleotide sequence encoding a polypeptide comprising at least 80%, 85%, 90%, 95%, 99%, or 100% sequence identity to an epitope sequence of SEQ ID NO: 5.

The composition can further comprise an isolated and purified plasmid comprising at least one nucleotide sequence encoding a polypeptide comprising at least 70%, 80%, 85%, 90%, 95%, 99%, or 100% sequence identity to an epitope sequence of SEQ ID NO: 8 (Zfp238). The composition can further comprise an isolated and purified plasmid comprising at least one nucleotide sequence encoding a polypeptide comprising at least 70%, 80%, 85%, 90%, 95%, 99%, or 100% sequence identity to an epitope sequence of SEQ ID NO: 10 (Lgals8). The composition can further comprise an isolated and purified plasmid comprising at least one nucleotide sequence encoding a polypeptide comprising at least 70%, 80%, 85%, 90%, 95%, 99%, or 100% sequence identity to an epitope sequence of SEQ ID NO: 12 (Vps35).

The composition can further comprise a nucleic acid polymer that hybridizes to a target sequence encoding Zfp238, Lgals8, or Vps35, wherein the nucleic acid polymer modulates the gene expression of the target sequence.

The composition can be formulated for subcutaneous, intramuscular, or intradermal administration. The composition can also be formulation for administration with an additional therapeutic agent, as a pharmaceutical composition or as a pharmaceutical combination.

### Diagnostic Methods

Methods for determining the presence and concentration levels of autoantibodies to pre-diagnostic antigens such as Otud6B, Pdhx, Stk39, Zfp238, Lgals8, and Vps35 are well known in the art. Exemplary methods include ELISA, radioimmunoassay (RIA), electrochemiluminescence (ECL), multiplexing technologies, or other similar methods.

For example, a disclosed autoantibody in a biological sample can be detected by means of a binding protein (e.g., a recombinant polypeptide described above) which is capable of interacting specifically with the disclosed autoantibody. In some cases, additional antibodies such as labeled secondary antibodies, binding portions thereof, or other binding partners are used for detection and quantification. The word “label” when used herein refers to a detectable compound or composition that is conjugated directly or indirectly to the antibody so as to generate a “labeled” antibody. In some embodiments, the label is detectable by itself (e.g., radioisotope labels or fluorescent labels) or, in the case of an enzymatic label, catalyzes chemical alteration of a substrate compound or composition that is detectable.

The additional antibodies (e.g., secondary antibodies) for detection of an autoantibody described herein can be either monoclonal or polyclonal in origin, or are synthetically or recombinantly produced. The amount of complexed protein, for example, the amount of autoantibody associated with the recombinant polypeptide and the amount of additional antibody to this complex, can be determined using standard protein detection methodologies known to those of skill in the art. A detailed review of immunological assay design, theory and protocols are found in numerous texts in the art (see, for example, Ausubel et al., eds. (1995) Current Protocols in Molecular Biology) (Greene Publishing and Wiley-Interscience, NY)); Coligan et al., eds. (1994) Current Protocols in Immunology (John Wiley & Sons, Inc., New York, N.Y.).

The choice of marker used to label the additional antibodies will vary depending upon the application. However, the choice of the marker is readily determinable to one skilled in the art. These labeled additional antibodies are used in immunoassays as well as in histological applications to detect the presence of an autoantibody of interest. The labeled additional antibodies are either polyclonal or monoclonal. Further, the additional antibodies for use in detecting an autoantibody of interest are labeled with a radioactive atom, an enzyme, a chromophoric or fluorescent moiety, or a colorimetric tag as described elsewhere herein. The choice of tagging label also will depend on the detection limitations desired. Enzyme assays (ELISAs) typically allow detection of a colored product formed by interaction of the enzyme-tagged complex with an enzyme substrate. Radionuclides that serve as detectable labels include, for example, I-131, I-123, I-125, Y-90, Re-188, Re-186, At-211, Cu-67, Bi-212, and Pd-109. Examples of enzymes that serve as detectable labels include, but are not limited to, horseradish peroxidase, alkaline phosphatase, beta-galactosidase, and glucose-6-phosphate dehydrogenase. Chromophoric moieties include, but are not limited to, fluorescein and rhodamine. The additional antibodies are conjugated to these labels by methods known in the art. For example, enzymes and chromophoric molecules are conjugated to the antibodies by means of coupling agents, such as dialdehydes, carbodiimides, dimaleimides, and the like. Alternatively, conjugation occurs through a ligand-receptor pair. Examples of suitable ligand-receptor pairs are biotin-avidin or biotin-streptavidin, and antibody-antigen.

The recombinant polypeptide can be either synthesized in a cell system or cell-free system. Suitable cell systems include prokaryotic and eukaryotic cell systems such as for example:

Prolaryotic cell lines such as E. coli cell lines comprising BL21 vector series, pBAD vector series, pBADM vector series, pET vector series, or pETM vector series;

Yeast cell lines such as Pichia pastoris yeast strains such as GS115, KM71H, SMD1168, SMD1168H, and X-33; and Saccharomyces cerevisiae yeast strain such as INVSc1;

Insect cell lines such as Drosophila S2 cells, Sf9 cells, Sf21 cells, High Five™ cells, and expresSF+® cells; and

Mammalian cell lines such as 293A cell line, 293FT cell line, 293F cells, 293 H cells, CHO DG44 cells, CHO-S cells, CHO-K1 cells, Flp-In™ T-REx™ 293 cell line, Flp-In™-293 cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

Suitable cell free systems can include systems such as Rapid Translation System RTS 100 (Roche), PURExprss® system (NEB), Expressway™ Expression System (ThermoFisher), 1-Step CHO High Yield IVT kit (ThermoFisher), and the like.

As described elsewhere herein, the sample for use in the methods can be from any fluid from a patient. Samples include, but are not limited, to whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract. In some cases, the sample is a blood serum sample. In some embodiments, the sample is a blood sample that is a venous, arterial, peripheral, tissue, cord blood sample.

The collection of a sample from the individual can be performed at regular intervals, such as, for example, one day, two days, three days, four days, five days, six days, one week, two weeks, weeks, four weeks, one month, two months, three months, four months, five months, six months, one year, daily, weekly, bimonthly, quarterly, biyearly or yearly.

### Pharmaceutical Compositions

The immunogenic compositions of the disclosure are preferably formulated as a vaccine for in vivo administration to the subject, such that they confer an antibody titer superior to the criterion for seroprotection for each antigenic component for an acceptable percentage of subjects. Antigens with an associated antibody titer above which a subject is considered to be seroconverted against the antigen are well known, and such titers are published by organizations such as WHO. In some instances, more than 80% of a statistically significant sample of subjects is seroconverted, more preferably more than 90%, still more preferably more than 93% and most preferably 96-100%.

### Adjuvants

The immunogenic compositions of the disclosure are preferably adjuvanted. An adjuvant can be used to enhance the immune response (humoral and/or cellular) elicited in a patient receiving the vaccine. Sometimes, adjuvants can elicit a T cell mediated immune response. The T cell mediated immune response can be a Th1-type response. The T cell mediated immune response can be a Th2-type response. A Th1-type response can be characterized by the production of cytokines such as IFN-γ as opposed to a Th2-type response which can be characterized by the production of cytokines such as IL-4, IL-5 and IL-10.

Adjuvant can comprise stimulatory molecules such as cytokines. Non-limiting examples of cytokines include: CCL20, α-interferon(IFN-a), β-interferon (IFN-β), γ-interferon, platelet derived growth factor (PDGF), TNFa, TNFp, granulocyte macrophage colony-stimulating factor (GM-CSF), epidermal growth factor (EGF), cutaneous T cell-attracting chemokine (CTACK), epithelial thymus-expressed chemokine (TECK), mucosae-associated epithelial chemokine (MEC), IL-12, IL-15, IL-28, MHC, CD80, CD86, IL-1, IL-2, IL-4, IL-5, IL-6, IL-10, IL-18, MCP-1, MIP-1a, MIP-1-, IL-8, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DRS, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-I, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAPI, and TAP2. In some instances, the adjuvant is GM-CSF.

Additional adjuvants include: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, IL-22, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

In some aspects, an adjuvant can be a modulator of a toll like receptor. Examples of modulators of toll-like receptors include TLR-9 agonists and are not limited to small molecule modulators of toll-like receptors such as Imiquimod. Other examples of adjuvants that are used in combination with a vaccine described herein can include and are not limited to saponin, CpG ODN and the like.

In some cases, an adjuvant can be a CD40 agonist. The CD40 agonist can be an antibody or fragments thereof or a small molecule. Exemplary CD40 agonists include: dacetuzmumab (SGN-40 or huS2C6 from Seattle Genetics), SEA-CD40 (Seattle Genetics), CP-870.893 (Pfizer), Chi Lob 7/4 (University of Southampton), or ADC-1013. Additional CD40 agonists can include those such as FGK-45 described in Medina-Echeverz et al., “Agonistic CD40 antibody induces immune-mediated liver damage and modulates tumor-induced myeloid suppressive cells” J. for ImmunoTherapy of Cancer 2(3):P174 (2014).

Sometimes, adjuvants may include an aluminum salt such as aluminum hydroxide gel (alum), aluminum phosphate, a salt of calcium, iron or zinc, or may be an insoluble suspension of acylated tyrosine, or acylated sugars, cationically or anionically derivatized polysaccharides, or polyphosphazenes.

Sometimes, suitable adjuvant systems which promote a predominantly Th1 response include. Monophosphoryl lipid A or a derivative thereof, particularly 3-de-O-acylated monophosphoryl lipid A, and a combination of monophosphoryl lipid A, preferably 3-de-O-acylated monophosphoryl lipid A (3D-MPL) together with an aluminum salt. An enhanced system involves the combination of a monophosphoryl lipid A and a saponin derivative, particularly the combination of QS21 and 3D-MPL as disclosed in WO 94/00153, or a less reactogenic composition where the QS21 is quenched with cholesterol as disclosed in WO 96/33739.

Sometimes, a suitable adjuvant system may include an adjuvant or immunostimulant such as but not limited to detoxified lipid A from any source and non-toxic derivatives of lipid A, saponins and other reagents capable of stimulating a Th1 type response. It has been known that enterobacterial lipopolysaccharide (LPS) is a potent stimulator of the immune system, although its use in adjuvants has been curtailed by its toxic effects. A non-toxic derivative of LPS, monophosphoryl lipid A (MPL), produced by removal of the core carbohydrate group and the phosphate from the reducing-end glucosamine, has been described by Ribi et al (1986, Immunology and hnmunopharmacology of bacterial endotoxins, Plenum Publ. Corp., NY. p407-419).

A further detoxified version of MPL results from the removal of the acyl chain from the 3-position of the disaccharide backbone, and is called 3-O-Deacylated monophosphoryl Lipid A (3D-MPL). It can be purified and prepared by the methods taught in GB 2122204B, which reference also discloses the preparation of diphosphoryl lipid A, and 3-O-deacylated variants thereof.

In some instances, 3D-MPL is in the form of an emulsion having a small particle size less than 0.2 μm in diameter, and its method of manufacture is disclosed in WO 94/21292. Aqueous formulations comprising monophosphoryl lipid A and a surfactant have been described in WO9843670A2. The bacterial lipopolysaccharide derived adjuvants to be formulated in the compositions of the present disclosure may be purified and processed from bacterial sources, or alternatively they may be synthetic. For example, purified monophosphoryl lipid A is described in Ribi et al 1986 (supra), and 3-O-Deacylated monophosphoryl or diphosphoryl lipid A derived from Salmonella sp. is described in GB 2220211 and U.S. Pat. No. 4,912,094. Other purified and synthetic lipopolysaccharides have been described (Hilgers et al, 1986. Int. ArchAllergy. Immunol, 79(4):392-6; Hilgers et al. 1987, Immunology. 60(1):141-6; and EP 0 549 074 BI). A particularly preferred bacterial lipopolysaccharide adjuvant is 3D-MPL.

Accordingly, the LPS derivatives that may be used in the present disclosure are those immunostimulants that are similar in structure to that of LPS or MPL or 3D-MPL. In another aspect of the present disclosure the LPS derivatives may be an acylated monosaccharide, which is a sub-portion to the above structure of MPL.

Saponins are taught in: Lacaille-Dubois, M and Wagner H. (1996. A review of the biological and pharmacological activities of saponins. Phytomedicine vol 2 pp 363-386). Saponins are steroid or triterpene glycosides widely distributed in the plant and marine animal kingdoms. Saponins are noted for forming colloidal solutions in water which foam on shaking, and for precipitating cholesterol. When saponins are near cell membranes they create pore-like structures in the membrane which cause the membrane to burst. Haemolysis of erythrocytes is an example of this phenomenon, which is a property of certain, but not all, saponins.

Saponins are known as adjuvants in vaccines for systemic administration. The adjuvant and haemolytic activity of individual saponins has been extensively studied in the art (Lacaille-Dubois and Wagner, supra). For example, Quil A (derived from the bark of the South American tree Quillaja Saponaria Molina), and fractions thereof, are described in U.S. Pat. No. 5,057,540 and “Saponins as vaccine adjuvants”, Kensil, C. R., CritRev TherDrug Carrier Syst, 1996, 12 (1-2):1-55; and EP 0362 279 B1.

Particulate structures, termed Immune Stimulating Complexes (ISCOMS), comprising fractions of Quil A are haemolytic and have been used in the manufacture of vaccines (Morein, B., EP 0 109 942 B1; WO 96/11711; WO 96/33739). The haemolytic saponins QS21 and QS17 (HPLC purified fractions of Quil A) have been described as potent systemic adjuvants, and the method of their production is disclosed in U.S. Pat. No. 5,057,540 and EP 0362279 B1. Other saponins which have been used in systemic vaccination studies include those derived from other plant species such as Gypsophila and Saponaria (Bomford et al., Vaccine, 10(9):572-577, 1992).

An enhanced system involves the combination of a non-toxic lipid A derivative and a saponin derivative particularly the combination of QS21 and 3D-MPL as disclosed in WO 94/00153, or a less reactogenic composition where the QS21 is quenched with cholesterol as disclosed in WO 96/33739.

Sometimes, an adjuvant is selected from bacteria toxoids, polyoxypropylene-polyoxyethylene block polymers, aluminum salts, liposomes, CpG polymers, oil-in-water emulsions, or a combination thereof.

Sometimes, an adjuvant is an oil-in-water emulsion. The oil-in-water emulsion can include at least one oil and at least one surfactant, with the oil(s) and surfactant(s) being biodegradable (metabolisable) and biocompatible. The oil droplets in the emulsion are generally less than 5 Lm in diameter, and may even have a sub-micron diameter, with these small sizes being achieved with a microfluidiser to provide stable emulsions. Droplets with a size less than 220 nm are preferred as they can be subjected to filter sterilization.

The oils used can include such as those from an animal (such as fish) or vegetable source. Sources for vegetable oils can include nuts, seeds and grains. Peanut oil, soybean oil, coconut oil, and olive oil, the most commonly available, exemplify the nut oils. Jojoba oil can be used e.g., obtained from the jojoba bean. Seed oils include safflower oil, cottonseed oil, sunflower seed oil, sesame seed oil, etc. The grain group can include: corn oil and oils of other cereal grains such as wheat, oats, rye, rice, teff, triticale, and the like. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk can be metabolizable and can therefore be used in with the vaccines described herein. The procedures for separation, purification, saponification and other means necessary for obtaining pure oils from animal sources are well known in the art. Fish can contain metabolizable oils which can be readily recovered. For example, cod liver oil, shark liver oils, and whale oil such as spermaceti can exemplify several of the fish oils which can be used herein. A number of branched chain oils can be synthesized biochemically in 5-carbon isoprene units and can be generally referred to as terpenoids. Shark liver oil contains a branched, unsaturated terpenoid known as squalene, 2,6,10,15,19,23-hexamethyl-2,6,10,14,18,22-tetracosahexaene. Squalane, the saturated analog to squalene, can also be used. Fish oils, including squalene and squalane, can be readily available from commercial sources or can be obtained by methods known in the art.

Other useful oils include tocopherols, can included in vaccines for use in elderly patients (e.g., aged 60 years or older) due to vitamin E been reported to have a positive effect on the immune response in this patient group. Further, tocopherols have antioxidant properties that can help to stabilize the emulsions. Various tocopherols exist (α, β, γ, δ, ε or ξ) but α is usually used. An example of α-tocopherol is DL-α-tocopherol, α-tocopherol succinate can be compatible with cancer vaccines and can be a useful preservative as an alternative to mercurial compounds.

Mixtures of oils can be used e.g. squalene and α-tocopherol. An oil content in the range of 2-20% (by volume) can be used.

Surfactants can be classified by their ‘HLB’ (hydrophile/lipophile balance). In some cases, surfactants have a HLB of at least 10, at least 15, and/or at least 16. Surfactants can include, but are not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Non-ionic surfactants can be used herein.

Mixtures of surfactants can be used e.g. Tween 80/Span 85 mixtures. A combination of a polyoxyethylene sorbitan ester and an octoxynol can also be suitable. Another combination can comprise laureth 9 plus a polyoxyethylene sorbitan ester and/or an octoxynol.

The amounts of surfactants (% by weight) can be: polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

Specific oil-in-water emulsion adjuvants include, but are not limited to:

A submicron emulsion of squalene, polysorbate 80, and sorbitan trioleate. The composition of the emulsion by volume can be about 5% squalene, about 0.5% polysorbate 80 and about 0.5% Span 85. In weight terms, these ratios become 4.3% squalene, 0.5% polysorbate 80 and 0.48% Span 85. This adjuvant is known as ‘MF59’. The MF59 emulsion advantageously includes citrate ions e.g., 10 mM sodium citrate buffer.

A submicron emulsion of squalene, a tocopherol, and polysorbate 80. These emulsions can have from 2 to 10% squalene, from 2 to 10% tocopherol and from 0.3 to 3% polysorbate 80, and the weight ratio of squalene:tocopherol is preferably ≦1 (e.g., 0.90) as this can provide a more stable emulsion. Squalene and polysorbate 80 may be present at a volume ratio of about 5:2 or at a weight ratio of about 11:5. One such emulsion can be made by dissolving Tween 80 in PBS to give a 2% solution, then mixing 90 ml of this solution with a mixture of (5 g of DL-α-tocopherol and 5 ml squalene), then microfluidising the mixture. The resulting emulsion has submicron oil droplets e.g., with an average diameter of between 100 and 250 nm, preferably about 180 nm. The emulsion may also include a 3-de-O-acylated monophosphoryl lipid A (3d-MPL). Another useful emulsion of this type may comprise, per human dose, 0.5-10 mg squalene, 0.5-11 mg tocopherol, and 0.1-4 mg polysorbate 80.

An emulsion of squalene, a tocopherol, and a Triton detergent (e.g., Triton X-100). The emulsion can also include a 3d-MPL (see below). The emulsion can contain a phosphate buffer.

An emulsion comprising a polysorbate (e.g., polysorbate 80), a Triton detergent (e.g., Triton X-100) and a tocopherol (e.g., an α-tocopherol succinate). The emulsion can include these three components at a mass ratio of about 75:11:10 (e.g., 750 μml polysorbate 80, 110 μml Triton X-100 and 100 μ/ml α-tocopherol succinate), and these concentrations should include any contribution of these components from antigens. The emulsion can also include squalene. The emulsion may also include a 3d-MPL. The aqueous phase can contain a phosphate buffer.

An emulsion of squalane, polysorbate 80 and poloxamer 401 (“Pluronic™ L121”). The emulsion can be formulated in phosphate buffered saline, pH 7.4. This emulsion can be a useful delivery vehicle for muramyl dipeptides, and can be used with threonyl-MDP in the “SAF-1” adjuvant (0.05-1% Thr-MDP, 5% squalane, 2.5% Pluronic L121 and 0.2% polysorbate 80). It can also be used without the Thr-MDP, as in the “AF” adjuvant (5% squalane, 1.25% Pluronic L121 and 0.2% polysorbate 80).

An emulsion comprising squalene, an aqueous solvent, a polyoxyethylene alkyl ether hydrophilic nonionic surfactant (e.g., polyoxyethylene (12) cetostearyl ether) and a hydrophobic nonionic surfactant (e.g., a sorbitan ester or mannide ester, such as sorbitan monoleate or ‘Span 80’). The emulsion can be thermoreversible and/or has at least 90% of the oil droplets (by volume) with a size less than 200 nm. The emulsion can also include one or more of: alditol; a cryoprotective agent (e.g., a sugar, such as dodecylmaltoside and/or sucrose); and/or an alkylpolyglycoside. The emulsion can include a TLR4 agonist. Such emulsions can be lyophilized.

An emulsion of squalene, poloxamer 105 and Abil-Care. The final concentration (weight) of these components in adjuvanted vaccines can be 5% squalene, 4% poloxamer 105 (pluronic polyol) and 2% Abil-Care 85 (Bis-PEG/PPG-16/16 PEG/PPG-16/16 dimethicone; caprylic/capric triglyceride).

An emulsion having from 0.5-50% of an oil, 0.1-10% of a phospholipid, and 0.05-5% of a non-ionic surfactant. Phospholipid components can include phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.

A submicron oil-in-water emulsion of a non-metabolisable oil (such as light mineral oil) and at least one surfactant (such as lecithin, Tween 80 or Span 80). Additives can include, QuilA saponin, cholesterol, a saponin-lipophile conjugate (such as GPI-0100, produced by addition of aliphatic amine to desacylsaponin via the carboxyl group of glucuronic acid), dimethyldioctadecylammonium bromide and/or N,N-dioctadecyl-N,N-bis (2-hydroxyethyl)propanediamine.

### Carriers and Excipients

In some instances, a composition described herein may further comprise carriers and excipients (including but not limited to buffers, carbohydrates, mannitol, proteins, polypeptides or amino acids such as glycine, antioxidants, bacteriostats, chelating agents, suspending agents, thickening agents and/or preservatives), water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, adjuvants, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel sodium stearate, glycerol monostearate, talc, sodium chloride, dried skim milk, glycerol, propylene, glycol, water, ethanol and the like. In another instances, the pharmaceutical preparation is substantially free of preservatives. In other instances, the pharmaceutical preparation can contain at least one preservative. General methodology on pharmaceutical dosage forms is found in Ansel et al., Pharmaceutical Dosage Forms and Drug Delivery Systems (Lippencott Williams & Wilkins, Baltimore Md. (1999)). It will be recognized that, while any suitable carrier known to those of ordinary skill in the art can be employed to administer the pharmaceutical compositions described herein, the type of carrier will vary depending on the mode of administration.

The composition may include a surfactant. Exemplary surfactants may include octylphenoxy polyoxyethanols and polyoxyethylene sorbitan esters, as described in “Surfactant Systems” Eds: Attwood and Florence (1983, Chapman and Hall). Octylphenoxy polyoxyethanols (the octoxynols), including t-octylphenoxypolyethoxyethanol (Triton X-100™) are also described in Merck Index Entry 6858 (Page 1162, 12th Edition, Merck & Co. Inc., Whitehouse Station. N.J., USA; ISBN 0911910-12-3). The polyoxyethylene sorbitan esters, including polyoxyethylene sorbitan monooleate (Tween 80™) are described in Merck Index Entry 7742 (Page 1308, 12th Edition. Merck & Co. Inc., Whitehouse Station, N.J., USA; ISBN 0911910-12-3). Both may be manufactured using methods described therein, or purchased from commercial sources such as Sigma Inc.

Exemplary non-ionic surfactants may include Triton X-45, t-octylphenoxy polyethoxyethanol (Triton X-100), Triton X-102, Triton X-114, Triton X-165, Triton X-205, Triton X-305, Trito-57, Triton-101, Trito-128, Breij 35, polyoxyethylene-9-lauryl ether (laureth 9) and polyoxyethylene-9-stearyl ether (steareth 9). Polyoxyethylene ethers may include polyoxyethylene-8-stearyl ether, polyoxyethylene-4-lauryl ether, polyoxyethylene-35-lauryl ether, and polyoxyethylene-23-lauryl ether.

Alternative terms or names for polyoxyethylene lauryl ether are disclosed in the CAS registry. The CAS registry number of polyoxyethylene-9 lauryl ether is: 9002-92-0. Polyoxyethylene ethers such as polyoxyethylene lauryl ether are described in the Merck index (12th ed: entry 7717, Merck & Co. Inc., Whitehouse Station, N.J., USA; ISBN 0911910-12-3). Laureth 9 is formed by reacting ethylene oxide with dodecyl alcohol and has an average of nine ethylene oxide units.

The ratio of the length of the polyoxyethylene section to the length of the alkyl chain in the surfactant (i.e., the ratio of n: alkyl chain length), affects the solubility of this class of surfactant in an aqueous medium. Thus, the surfactants of the present disclosure may be in solution or may form particulate structures such as micelles or vesicles. As a solution, the surfactants of the present disclosure are safe, easily sterilisable, simple to administer, and maybe manufactured in a simple fashion without the GMP and QC issues associated with the formation of uniform particulate structures. Some polyoxyethylene ethers, such as laureth 9, are capable of forming non-vesicular solutions. However, polyoxyethylene-8 palmitoyl ether (C18E8) is capable of forming vesicles. Accordingly, vesicles of polyoxyethylene-8 palmitoyl ether in combination with at least one additional non-ionic surfactant, can be employed in the formulations of the present disclosure.

Within the inherent experimental variability of such a biological assay, the polyoxyethylene ethers, or surfactants of general formula (I), of the present disclosure preferably have a haemolytic activity, of approximately between 0.5-0.0001%, more preferably between 0.05-0.0001%, even more preferably between 0.005-0.0001%, and most preferably between 0.003-0.0004%. Ideally, said polyoxyethylene ethers or esters should have a haemolytic activity similar (i.e., within a ten-fold difference) to that of either polyoxyethylene-9 lauryl ether or polyoxyethylene-8 stearyl ether.

Two or more non-ionic surfactants from the different groups of surfactants described may be present in the vaccine formulation described herein. In particular, a combination of a polyoxyethylene sorbitan ester such as polyoxyethylene sorbitan monooleate (Tween 80™) and an octoxynol such as t-octylphenoxypolyethoxyethanol (Triton) X-100™ is preferred. Another particularly preferred combination of non-ionic surfactants comprises laureth 9 plus a polyoxyethylene sorbitan ester or an octoxynol or both.

Preferably each non-ionic surfactant is present in the final vaccine formulation at a concentration of between 0.001 to 20%, more preferably 0.01 to 10%, and most preferably up to about 2% (w/v). Where one or two surfactants are present, these are generally present in the final formulation at a concentration of up to about 2% each, typically at a concentration of up to about 0.6% each. One or more additional surfactants may be present, generally up to a concentration of about 1% each and typically in traces up to about 0.2% or 0.1% each. Any mixture of surfactants may be present in the vaccine formulations according to the disclosure. Non-ionic surfactants such as those discussed above have preferred concentrations in the final vaccine composition as follows: polyoxyethylene sorbitan esters such as Tween 80™: 0.01 to 1%, most preferably about 0.1% (w/v); octyl- or nonylphenoxy polyoxyethanols such as Triton X-100™ or other detergents in the Triton series: 0.001 to 0.1%, most preferably 0.005 to 0.02% (w/v); polyoxyethylene ethers of general formula (I) such as laureth 9: 0.1 to 20%, preferably 0.1 to 10% and most preferably 0.1 to 1% or about 0.5% (w/v).

A composition may be encapsulated within liposomes using well-known technology. Biodegradable microspheres can also be employed as carriers for the pharmaceutical compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344 and 5,942,252.

The composition may be administered in liposomes or microspheres (or microparticles). Methods for preparing liposomes and microspheres for administration to a patient are well known to those of skill in the art. U.S. Pat. No. 4,789,734, the contents of which are hereby incorporated by reference, describes methods for encapsulating biological materials in liposomes. Essentially, the material is dissolved in an aqueous solution, the appropriate phospholipids and lipids added, along with surfactants if required, and the material dialyzed or sonicated, as necessary. A review of known methods is provided by G. Gregoriadis. Chapter 14, “Liposomes,” Drug Carriers in Biology and Medicine, pp. 2.sup 87-341 (Academic Press, 1979).

Microspheres formed of polymers or proteins are well known to those skilled in the art, and can be tailored for passage through the gastrointestinal tract directly into the blood stream. Alternatively, the compound can be incorporated and the microspheres, or composite of microspheres, implanted for slow release over a period of time ranging from days to months. See, for example, U.S. Pat. Nos. 4,906,474, 4,925,673 and 3,625,214, and Jein, TIPS 19:155-157 (1998), the contents of which are hereby incorporated by reference.

A composition may include preservatives such as thiomersal or 2-phenoxyethanol. In some instances, the vaccine is substantially free from (e.g., <10 g/ml) mercurial material e.g., thiomersal-free α-Tocopherol succinate may be used as an alternative to mercurial compounds.

For controlling the tonicity, a physiological salt such as sodium salt can be included in the vaccine. Other salts can include potassium chloride, potassium dihydrogen phosphate, disodium phosphate, and/or magnesium chloride, or the like.

A composition may have an osmolality of between 200 mOsm/kg and 400 mOsm/kg, between 240-360 mOsm/kg, or within the range of 290-310 mOsm/kg.

A composition may comprise one or more buffers, such as a Tris buffer; a borate buffer; a succinate buffer; a histidine buffer (particularly with an aluminum hydroxide adjuvant); or a citrate buffer. Buffers, in some cases, are included in the 5-20 mM range.

The pH of the composition may be between about 5.0 and about 8.5, between about 6.0 and about 8.0, between about 6.5 and about 7.5, or between about 7.0 and about 7.8.

A composition may be sterile. The vaccine can be non-pyrogenic e.g. containing <1 EU (endotoxin unit, a standard measure) per dose, and can be <0.1 EU per dose. The composition can be gluten free.

A composition may include detergent e.g. a polyoxyethylene sorbitan ester surfactant (known as ‘Tweens’), an octoxynol (such as octoxynol-9 (Triton X-100) or t-octylphenoxypolyethoxyethanol), a cetyl trimethyl ammonium bromide (‘CTAB’), or sodium deoxycholate, particularly for a split or surface antigen vaccine. The detergent can be present only at trace amounts. Thus the vaccine can include less than 1 mg/ml of each of octoxynol-10 and polysorbate 80. Other residual components in trace amounts can be antibiotics (e.g. neomycin, kanamycin, polymyxin B).

A composition may be formulated as a sterile solution or suspension, in suitable vehicles, well known in the art. The pharmaceutical compositions may be sterilized by conventional, well-known sterilization techniques, or may be sterile filtered. The resulting aqueous solutions may be packaged for use as is, or lyophilized, the lyophilized preparation being combined with a sterile solution prior to administration. Suitable formulations and additional carriers are described in Remington “The Science and Practice of Pharmacy” (20^{th} Ed., Lippincott Williams & Wilkins, Baltimore Md.), the teachings of which are incorporated by reference in their entirety herein.

A composition may be formulated with one or more pharmaceutically acceptable salts. Pharmaceutically acceptable salts can include those of the inorganic ions, such as, for example, sodium, potassium, calcium, magnesium ions, and the like. Such salts can include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the agent(s) contain a carboxy group or other acidic group, it can be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine, triethanolamine, and the like.

Additional salts may comprise a bile acid or a derivative thereof. These include derivatives of cholic acid and salts thereof, in particular sodium salts of cholic acid or cholic acid derivatives. Examples of bile acids and derivatives thereof include cholic acid, deoxycholic acid, chenodeoxycholic acid, lithocholic acid, ursodeoxycholic acid, hyodeoxycholic acid and derivatives such as glyco-, tauro-, amidopropyl-1-propanesulfonic-, amidopropyl-2-hydroxy-1-propanesulfonic derivatives of the aforementioned bile acids, or N,N-bis (3Dgluconoamidopropyl) deoxycholamide. A particularly preferred example is sodium deoxycholate (NaDOC) which may be present in the final vaccine dose.

A composition described herein may further be bound to any nucleic acid molecule, such as a peptide, an antisense molecule, or other chemicals to facilitate delivery to a site of interest. In the context of the nucleic acid polymer described above, the nucleic acid polymer may be tethered to a protein, a peptide, an antisense molecule, or other chemicals to facilitate delivery. In some instances, the other chemicals include non-peptide or nucleic acid based molecules, such as a small molecule (e.g., a drug). Sometimes, the small molecule has a MW of less than 900 Da, 800 Da, 700 Da, 600 Da, 500 Da, 400 Da, or 300 Da.

A composition described herein may further be formulated with a cell penetrating peptide (CPP) for delivery to a site to interest. A skilled person will understand that any suitable CPP may be conjugated with the composition described herein (e.g., a nucleic acid polymer) to aid delivery of the composition to a site of interest. Such CPPs may be any suitable CPP technology described by Boisguërin et al, (2015), Advanced Drug Delivery Reviews doi: 10.1016/j.addr.2015.02.008), which is herein incorporated by reference. Suitable delivery vehicles for conjugation to the composition are also described in Lochmann et al ((European Journal of Pharmaceutics and Biopharmaceutics 58 (2004) 237-251), which is herein incorporated by reference).

The CPP may be an arginine and/or lysine rich peptide, for example, wherein the majority of residues in the peptide is either lysine or arginine. The CPP may comprise a poly-L-lysine (PLL). Alternatively, the CPP may comprise a poly-arginine. Suitable CPPs may be selected from the group comprising Penetratin; R6-Penetratin; Transportan; oligo-arginines; F-3; B-peptide; B-MSP; Pip peptides, such as Pip1, Pip2a, Pip2b, Pip5e, Pip5f, Pip5h, Pip5j; Pip5k, Pip51, Pip5m, Pip5n, Pip5o, Pip6a, Pip6b, Pip6c, Pip6d, Pip6e, Pip6f, Pip6g, or Pip6h; peptide of sequence PKKKRKV; Penatratin; Lys₄; SPACE; Tat; Tat-DRBD (dsRNA-binding domain); (RXR)₄; (RFF)₃RXB; (KFF)₃K; R_{g}F₂; T-cell derived CPP; Pep-3; PEGpep-3; MPG-8; MPG-8-Chol; PepFect6; P5RHH; R₁₅; and Chol-R₉; or functional variants thereof (e.g. see Boisguérin et al, (2015), Advanced Drug Delivery Reviews doi: 10.1016/j.addr.2015.02.008).

In some instances, the CPP comprises or consists of a Pip peptide. The Pip peptide may be selected from the group comprising Pip1, Pip2a, Pip2b, Pip5e, Pip5f, Pip5h, Pip5j; Pip5k, Pip51, Pip5m, Pip5n, Pip5o, Pip6a, Pip6b, Pip6c, Pip6d, Pip6c, Pip6f, Pip6g, and Pip6h.

In some cases, the delivery vehicle may comprise a peptide-based nanoparticle (PBN), wherein a plurality of CPPs (for example one or more suitable CPPs discussed herein) form a complex with the composition (e.g., the nucleic acid polymer) through charge interactions. Such nanoparticles may be between about 50 nm and 250 nm in size. In some instances, the nanoparticles may be about 70-200 nm in size. In other instances, the nanoparticles may be about 70-100 nm in size or 125-200 nm in size.

A composition comprising an active agent such as a peptide or a nucleic acid described herein, in combination with one or more adjuvants may be formulated in conventional manner using one or more physiologically acceptable carriers, comprising excipients, diluents, and/or auxiliaries, e.g., which facilitate processing of the active agents into preparations that can be administered. Proper formulation may depend at least in part upon the route of administration chosen. The agent(s) described herein may be delivered to a patient using a number of routes or modes of administration, including oral, buccal, topical, rectal, transdermal, transmucosal, subcutaneous, intravenous, and intramuscular applications, as well as by inhalation.

The active agents may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol.

For injectable formulations, the vehicle may be chosen from those known in the art to be suitable, including aqueous solutions or oil suspensions, or emulsions, with sesame oil, corn oil, cottonseed oil, or peanut oil, as well as elixirs, mannitol, dextrose, or a sterile aqueous solution, and similar pharmaceutical vehicles. The formulation may also comprise polymer compositions which are biocompatible, biodegradable, such as poly(lactic-co-glycolic)acid. These materials may be made into micro or nanospheres, loaded with drug and further coated or derivatized to provide superior sustained release performance. Vehicles suitable for periocular or intraocular injection include, for example, suspensions of therapeutic agent in injection grade water, liposomes and vehicles suitable for lipophilic substances. Other vehicles for periocular or intraocular injection are well known in the art.

In some instances, a composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it may be dispensed with an infusion bottle containing sterile pharmaceutical grade water or saline. Where the composition is administered by injection, an ampoule of sterile water for injection or saline can be provided so that the ingredients may be mixed prior to administration.

When administration is by injection, the active agent may be formulated in aqueous solutions, specifically in physiologically compatible buffers such as Hanks solution. Ringer's solution, or physiological saline buffer. The solution may contain formulatory agents such as suspending, stabilizing and/or dispersing agents. Alternatively, the active compound may be in powder form for constitution with a suitable vehicle, e.g., sterile pyrogen-free water, before use. In another embodiment, the pharmaceutical composition does not comprise an adjuvant or any other substance added to enhance the immune response stimulated by the peptide. In another embodiment, the pharmaceutical composition comprises a substance that inhibits an immune response to the peptide. Methods of formulation are known in the art, for example, as disclosed in Remington's Pharmaceutical Sciences, latest edition, Mack Publishing Co., Easton P.

In addition to the formulations described above, the active agents may also be formulated as a depot preparation. Such long acting formulations may be administered by implantation or transcutaneous delivery (for example subcutaneously or intramuscularly), intramuscular injection or use of a transdermal patch. Thus, for example, the agents may be formulated with suitable polymeric or hydrophobic materials (for example as an emulsion in an acceptable oil) or ion exchange resins, or as sparingly soluble derivatives, for example, as a sparingly soluble salt.

In some cases, compositions comprising one or more agents exert local and regional effects when administered topically or injected at or near particular sites of infection. Direct topical application, e.g., of a viscous liquid, solution, suspension, dimethylsulfoxide (DMSO)-based solutions, liposomal formulations, gel, jelly, cream, lotion, ointment, suppository, foam, or aerosol spray, can be used for local administration, to produce for example local and/or regional effects. Pharmaceutically appropriate vehicles for such formulation include, for example, lower aliphatic alcohols, polyglycols (e.g., glycerol or polyethylene glycol), esters of fatty acids, oils, fats, silicones, and the like. Such preparations can also include preservatives (e.g., p-hydroxybenzoic acid esters) and/or antioxidants (e.g., ascorbic acid and tocopherol). See also Dermatological Formulations: Percutaneous absorption, Barry (Ed.), Marcel Dekker Incl, 1983. In another embodiment, local/topical formulations comprising a transporter, carrier, or ion channel inhibitor are used to treat epidermal or mucosal viral infections.

Compositions may contain a cosmetically or dermatologically acceptable carrier. Such carriers are compatible with skin, nails, mucous membranes, tissues and/or hair, and can include any conventionally used cosmetic or dermatological carrier meeting these requirements. Such carriers can be readily selected by one of ordinary skill in the art. In formulating skin ointments, an agent or combination of agents can be formulated in an oleaginous hydrocarbon base, an anhydrous absorption base, a water-in-oil absorption base, an oil-in-water water-removable base and/or a water-soluble base. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. The construction and use of transdermal patches for the delivery of pharmaceutical agents is well known in the art. See, e.g., U.S. Pat. Nos. 5,023,252, 4,992,445 and 5,001,139. Such patches can be constructed for continuous, pulsatile, or on demand delivery of pharmaceutical agents.

Lubricants which may be used to form pharmaceutical compositions and dosage forms can include, but are not limited to, calcium stearate, magnesium stearate, mineral oil, light mineral oil, glycerin, sorbitol, mannitol, polyethylene glycol other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

The compositions may be in any form suitable for topical application, including aqueous, aqueous-alcoholic or oily solutions, lotion or serum dispersions, aqueous, anhydrous or oily gels, emulsions obtained by dispersion of a fatty phase in an aqueous phase (O/W or oil in water) or, conversely. (W/O or water in oil), microemulsions or alternatively microcapsules, microparticles or lipid vesicle dispersions of ionic and/or nonionic type. These compositions may be prepared according to conventional methods. Other than the agents of the invention, the amounts of the various constituents of the compositions according to the invention are those conventionally used in the art. These compositions in particular constitute protection, treatment or care creams, milks, lotions, gels or foams for the face, for the hands, for the body and/or for the mucous membranes, or for cleansing the skin. The compositions can also consist of solid preparations constituting soaps or cleansing bars.

Compositions may contain adjuvants such as hydrophilic or lipophilic gelling agents, hydrophilic or lipophilic active agents, preserving agents, antioxidants, solvents, fragrances, fillers, sunscreens, odor-absorbers and dyestuffs. The amounts of these various adjuvants are those conventionally used in the fields considered and, for example, are from about 0.01% to about 20% of the total weight of the composition. Depending on their nature, these adjuvants can be introduced into the fatty phase, into the aqueous phase and/or into the lipid vesicles.

For oral administration, the active agent(s) may be formulated readily by combining the active agent(s) with pharmaceutically acceptable carriers well known in the art. Such carriers enable the agents of the invention to be formulated as tablets, including chewable tablets, pills, dragees, capsules, lozenges, hard candy, liquids, gels, syrups, slurries, powders, suspensions, elixirs, wafers, and the like, for oral ingestion by a patient to be treated. Such formulations can comprise pharmaceutically acceptable carriers including solid diluents or fillers, sterile aqueous media and various non-toxic organic solvents. A solid carrier may be one or more substances which can also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets contain from about one (1) to about seventy (70) percent of the active compound. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Generally, the active agents may be included at concentration levels ranging from about 0.5%, about 5%, about 10%, about 20%, or about 30% to about 50%, about 60%, about 70%, about 80% or about 90% by weight of the total composition of oral dosage forms, in an amount sufficient to provide a desired unit of dosage.

Aqueous suspensions for oral use may contain active agent(s) with pharmaceutically acceptable excipients, such as a suspending agent (e.g., methyl cellulose), a wetting agent (e.g., lecithin, lysolecithin and/or a long-chain fatty alcohol), as well as coloring agents, preservatives, flavoring agents, and the like.

Oils or non-aqueous solvents can be required to bring the active agents into solution, due to, for example, the presence of large lipophilic moieties. Alternatively, emulsions, suspensions, or other preparations, for example, liposomal preparations, can be used. With respect to liposomal preparations, any known methods for preparing liposomes for treatment of a condition can be used. See, for example, Bangham et al., J. Mol. Biol. 23: 238-252 (1965) and Szoka et al:, Proc. Natl. Acad. Sci. USA 75: 4194-4198 (1978), incorporated herein by reference. Ligands can also be attached to the liposomes to direct these compositions to particular sites of action.

Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; flavoring elements, cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents can be added, such as the cross-linked polyvinyl pyrrolidone, agar, or alginic acid or a salt thereof such as sodium alginate. The agents can also be formulated as a sustained release preparation.

Dragee cores may be provided with suitable coatings. For this purpose, concentrated sugar solutions can be used, which can optionally contain gum arabic, talc, polyvinyl pyrrolidone, carbopol gel, polyethylene glycol and/or titanium dioxide, lacquer solutions, and suitable organic solvents or solvent mixtures. Dyestuffs or pigments can be added to the tablets or dragee coatings for identification or to characterize different combinations of active agents.

Pharmaceutical preparations that may be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, and/or lubricants such as talc or magnesium stearate and, optionally, stabilizers. In soft capsules, the active agents may be dissolved or suspended in suitable liquids, such as fatty oils, liquid paraffin, or liquid polyethylene glycols. In addition, stabilizers can be added. All formulations for oral administration may be in dosages suitable for administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions may be prepared in solutions, for example, in aqueous propylene glycol solutions or can contain emulsifying agents, for example, such as lecithin, sorbitan monooleate, or acacia. Aqueous solutions may be prepared by dissolving the active component in water and adding suitable colorants, flavors, stabilizers, and thickening agents. Aqueous suspensions may be prepared by dispersing the finely divided active component in water with viscous material, such as natural or synthetic gums, resins, methylcellulose, sodium carboxymethylcellulose, and other well known suspending agents. Suitable fillers or carriers with which the compositions may be administered include agar, alcohol fats, lactose, starch, cellulose derivatives, polysaccharides, polyvinylpyrrolidone, silica, sterile saline and the like, or mixtures thereof used in suitable amounts. Solid form preparations include solutions, suspensions, and emulsions, and can contain, in addition to the active component, colorants, flavors, stabilizers, buffers, artificial and natural sweeteners, dispersants, thickeners, solubilizing agents, and the like.

A syrup or suspension may be made by adding the active compound to a concentrated, aqueous solution of a sugar, e.g., sucrose, to which can also be added any accessory ingredients. Such accessory ingredients may include flavoring, an agent to retard crystallization of the sugar or an agent to increase the solubility of any other ingredient, e.g., as a polyhydric alcohol, for example, glycerol or sorbitol.

When formulating compounds for oral administration, it may be desirable to utilize gastroretentive formulations to enhance absorption from the gastrointestinal (GI) tract. A formulation which is retained in the stomach for several hours may release compounds of the invention slowly and provide a sustained release that can be used herein. Disclosure of such gastroretentive formulations are found in Klausner, E. A.; Lavy, E.; Barta, M.; Cserepes, E.; Friedman, M.; Hoffman, A, 2003 “Novel gastroretentive dosage forms; evaluation of gastroretentivity and its effect on levodopa in humans.” Pharm. Res. 20, 1466-73. Hoffman, A.; Stepensky, D.; Lavy, E.; Eyal, S. Klausner, E.; Friedman, M. 2004 “Pharmacokinetic and pharmacodynamic aspects of gastroretentive dosage forms” Int. J. Pharm. 11, 141-53, Streubel, A.; Siepmann, J; Bodmeier, R.; 2006 “Gastroretentive drug delivery systems” Expert Opin. Drug Deliver. 3, 217-3, and Chavanpatil, M. D.; Jain, P.; Chaudhari, S.; Shear, R.; Vavia, P. R. “Novel sustained release, swellable and bioadhesive gastroretentive drug delivery system for olfoxacin” Int. J. Pharm. 2006 epub March 24. Expandable, floating and bioadhesive techniques can be utilized to maximize absorption of the compounds of the invention.

The solubility of the components of the compositions may be enhanced by a surfactant or other appropriate co-solvent in the composition. Such cosolvents include polysorbate 20, 60, and 80. Pluronic F68, F-84 and P-103, cyclodextrin, or other agents known to those skilled in the art. Such co-solvents can be employed at a level of from about 0.01% to 2% by weight.

The compositions may be packaged in multidose form. Preservatives may be preferred to prevent microbial contamination during use. Suitable preservatives include: benzalkonium chloride, thimerosal, chlorobutanol methyl paraben, propyl paraben, phenylethyl alcohol, edetate disodium, sorbic acid, Onamer M, or other agents known to those skilled in the art. In the prior art ophthalmic products, such preservatives can be employed at a level of from 0.004% to 0.02%. In the compositions of the present application the preservative, preferably benzalkonium chloride, can be employed at a level of from 0.001% to less than 0.01%, e.g. from 0.001% to 0.008%, preferably about 0.005% by weight. It has been found that a concentration of benzalkonium chloride of 0.005% can be sufficient to preserve the compositions of the present invention from microbial attack.

In instances relating to topical/local application, the compositions may include one or more penetration enhancers. For example, the formulations may comprise suitable solid or gel phase carriers or excipients that increase penetration or help delivery of agents or combinations of agents of the invention across a permeability barrier, e.g., the skin. Many of these penetration-enhancing compounds are known in the art of topical formulation, and include, e.g., water, alcohols (e.g., terpenes like methanol ethanol, 2-propanol), sulfoxides (e.g., dimethyl sulfoxide, decylmethyl sulfoxide, tetradecylmethyl sulfoxide), pyrrolidones (e.g, 2-pyrrolidone, N-methyl-2-pyrrolidone, N-(2-hydroxyethyl)pyrrolidone), laurocapram, acetone, dimethylacetamide, dimethylformamide, tetrahydrofurfuryl alcohol, L-α-amino acids, anionic, cationic, amphoteric or nonionic surfactants (e.g., isopropyl myristate and sodium lauryl sulfate), fatty acids, fatty alcohols (e.g., oleic acid), amines, amides, clofibric acid amides, hexamethylene lauramide, proteolytic enzymes, α-bisabolol d-limonene, urea and N,N-diethyl-m-toluamide, and the like. Additional examples include humectants (e.g., urea), glycols (e.g., propylene glycol and polyethylene glycol), glycerol monolaurate, alkanes, alkanols, ORGELASE, calcium carbonate, calcium phosphate, various sugars, starches, cellulose derivatives, gelatin, and/or other polymers. In another embodiment, the compositions may include one or more such penetration enhancers.

The compositions for local/topical application may include one or more antimicrobial preservatives such as quaternary ammonium compounds, organic mercurials, p-hydroxy benzoates, aromatic alcohols, chlorobutanol, and the like.

The compositions may be formulated into aerosol solutions, suspensions or dry powders. The aerosol can be administered through the respiratory system or nasal passages. For example, one skilled in the art will recognize that a composition of the present invention can be suspended or dissolved in an appropriate carrier, e.g., a pharmaceutically acceptable propellant, and administered directly into the lungs using a nasal spray or inhalant. For example, an aerosol formulation comprising a transporter, carrier, or ion channel inhibitor can be dissolved, suspended or emulsified in a propellant or a mixture of solvent and propellant, e.g., for administration as a nasal spray or inhalant. Aerosol formulations can contain any acceptable propellant under pressure, such as a cosmetically or dermatologically or pharmaceutically acceptable propellant, as conventionally used in the art.

An aerosol formulation for nasal administration is generally an aqueous solution designed to be administered to the nasal passages in drops or sprays. Nasal solutions can be similar to nasal secretions in that they are generally isotonic and slightly buffered to maintain a pH of about 5.5 to about 6.5, although pH values outside of this range may additionally be used. Antimicrobial agents or preservatives may also be included in the formulation.

An aerosol formulation for inhalations and inhalants may be designed so that the agent or combination of agents is carried into the respiratory tree of the subject when administered by the nasal or oral respiratory route. Inhalation solutions may be administered, for example, by a nebulizer. Inhalations or insufflations, comprising finely powdered or liquid drugs, may be delivered to the respiratory system as a pharmaceutical aerosol of a solution or suspension of the agent or combination of agents in a propellant, e.g., to aid in disbursement. Propellants may be liquefied gases, including halocarbons, for example, fluorocarbons such as fluorinated chlorinated hydrocarbons, hydrochlorofluorocarbons, and hydrochlorocarbons, as well as hydrocarbons and hydrocarbon ethers.

Halocarbon propellants may include fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Halocarbon propellants are described in Johnson, U.S. Pat. No. 5,376,359, issued Dec. 27, 1994; Byron et al., U.S. Pat. No. 5,190,029, issued Mar. 2, 1993; and Purewal et al., U.S. Pat. No. 5,776,434, issued Jul. 7, 1998. Hydrocarbon propellants useful in the invention include, for example, propane, isobutane, n-butane, pentane, isopentane and neopentane. A blend of hydrocarbons may also be used as a propellant. Ether propellants include, for example, dimethyl ether as well as the ethers. An aerosol formulation of the invention may also comprise more than one propellant. For example, the aerosol formulation may comprise more than one propellant from the same class, such as two or more fluorocarbons; or more than one, more than two, more than three propellants from different classes, such as a fluorohydrocarbon and a hydrocarbon. Pharmaceutical compositions of the present invention may also be dispensed with a compressed gas, e.g., an inert gas such as carbon dioxide, nitrous oxide or nitrogen.

Aerosol formulations may also include other components, for example, ethanol, isopropanol, propylene glycol, as well as surfactants or other components such as oils and detergents. These components may serve to stabilize the formulation and/or lubricate valve components.

The aerosol formulation may be packaged under pressure and can be formulated as an aerosol using solutions, suspensions, emulsions, powders and semisolid preparations. For example, a solution aerosol formulation may comprise a solution of an agent of the invention such as a transporter, carrier, or ion channel inhibitor in (substantially) pure propellant or as a mixture of propellant and solvent. The solvent may be used to dissolve the agent and/or retard the evaporation of the propellant. Solvents may include, for example, water, ethanol and glycols. Any combination of suitable solvents may be use, optionally combined with preservatives, antioxidants, and/or other aerosol components.

An aerosol formulation may be a dispersion or suspension. A suspension aerosol formulation may comprise a suspension of an agent or combination of agents of the instant invention, e.g., a transporter, carrier, or ion channel inhibitor, and a dispersing agent. Dispersing agents may include, for example, sorbitan trioleate, oleyl alcohol oleic acid, lecithin and corn oil. A suspension aerosol formulation may also include lubricants, preservatives, antioxidant, and/or other aerosol components.

An aerosol formulation may similarly be formulated as an emulsion. An emulsion aerosol formulation may include, for example, an alcohol such as ethanol, a surfactant, water and a propellant, as well as an agent or combination of agents of the invention, e.g., a transporter, carrier, or ion channel. The surfactant used may be nonionic, anionic or cationic. One example of an emulsion aerosol formulation comprises, for example, ethanol, surfactant, water and propellant. Another example of an emulsion aerosol formulation comprises, for example, vegetable oil, glyceryl monostearate and propane.

The compounds may be formulated for administration as suppositories. A low melting wax, such as a mixture of triglycerides, fatty acid glycerides, Witepsol S55 (trademark of Dynamite Nobel Chemical, Germany), or cocoa butter is first melted and the active component is dispersed homogeneously, for example, by stirring. The molten homogeneous mixture is then poured into convenient sized molds, allowed to cool, and to solidify.

The compounds may be formulated for vaginal administration. Pessaries, tampons, creams, gels, pastes, foams or sprays containing in addition to the active ingredient such carriers as are known in the art to be appropriate.

The compounds may be attached releasably to biocompatible polymers for use in sustained release formulations on, in or attached to inserts for topical, intraocular, periocular, or systemic administration. The controlled release from a biocompatible polymer can be utilized with a water soluble polymer to form an instillable formulation, as well. The controlled release from a biocompatible polymer, such as for example. PLGA microspheres or nanospheres, may be utilized in a formulation suitable for intra ocular implantation or injection for sustained release administration, as well. Any suitable biodegradable and biocompatible polymer may be used.

### Dosages, Routes of Administration, and Therapeutic Regimens

The compositions and methods described herein may elicit an immune response to an epitope of an antigenic peptide in a subject. In some cases, the compositions may be breast cancer vaccines. In some cases, the breast cancer vaccine may be a multiantigen breast cancer vaccine.

In some cases, the subject may be tumor bearing prior to administration of the vaccine. In other cases, the subject may not be tumor bearing prior to administration of the vaccine. In other cases, the subject may not be tumor bearing prior to administration of the vaccine but become tumor bearing after administration of the vaccine. In other cases, the subject may not be tumor bearing prior to administration of the vaccine and may not become tumor bearing after administration of the vaccine. In some instances, the tumors may be breast cancer tumors. Sometimes, the breast cancer tumors in humans can be triple negative tumors in humans.

The vaccine described herein may be delivered via a variety of routes. Delivery routes may include oral (including buccal and sub-lingual), rectal, nasal, topical, transdermal patch, pulmonary, vaginal, suppository, or parenteral (including intramuscular, intraarterial, intrathecal, intradermal, intraperitoneal, subcutaneous and intravenous) administration or in a form suitable for administration by aerosolization, inhalation or insufflation. General information on drug delivery systems can be found in Ansel et al., Pharmaceutical Dosage Forms and Drug Delivery Systems (Lippencott Williams & Wilkins, Baltimore Md. (1999)). The vaccine described herein can be administered to muscle, or can be administered via intradermal or subcutaneous injections, or transdermally, such as by iontophoresis. Epidermal administration of the vaccine can be employed.

In some instances, the vaccine may also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The vaccine may be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion.

The vaccine may include material for a single immunization, or may include material for multiple immunizations (i.e. a ‘multidose’ kit). The inclusion of a preservative is preferred in multidose arrangements. As an alternative (or in addition) to including a preservative in multidose compositions, the compositions can be contained in a container having an aseptic adaptor for removal of material.

The vaccine may be administered in a dosage volume of about 0.1, 0.2, 0.3, 0.4, 0.5, 0.6, 0.7, 0.8, 0.9, or 1.0 mL. Sometimes the vaccine can be administered in a higher dose e.g., of more than 1 ml.

In some cases, the subject may be immunized with one dose of the vaccine. In other cases, the subject may be immunized with more than one dose of the vaccine. For example, the subject may be immunized with more than one, more than two, more than three, more than four, more than five, more than six, more than seven, more than eight, more than nine, more than ten, more than 11, more than 12, more than 13, more than 14, more than 15, more than 16, more than 17, more than 18, more than 19 or more than 20 doses of the vaccine. In an exemplary case, the subject is immunized with three doses of the vaccine.

In the cases that a subject receives more than one dose of the vaccine, time may elapse between the first dose and each subsequent dose of the vaccine. In some cases, the time that elapses between the first dose an each subsequent dose of the vaccine may be seconds, minutes, hours, days, weeks, months or years. For example, more than one dose may be administered to the subject by intervals. In some cases, the intervals may occur over seconds, minutes, hours, days, weeks, months or years. In some cases, subjects may receive a booster dose. For example, the booster may be administered to the subject more than one, more than two, more than three, more than four, more than five, more than six, more than seven, more than eight, more than nine, more than ten, more than 11, more than 12, more than 13, more than 14, more than 15, more than 16, more than 17, more than 18, more than 19 or more than 20 booster doses of the vaccine. In an exemplary case, the subject may receive up to three boosters of the vaccine.

In some cases, intervals may be the same between each dose of the vaccine. In some cases, intervals may be the same between each booster of the vaccine. In some cases, intervals may be different between each dose of the vaccine. In some cases, intervals may be different between each booster of the vaccine.

In an exemplary case, more than one dose is administered to the subject over an interval of at least one day. In some cases, the interval may be a one day, two day, three day, four day, five day, six day, seven day, eight day, nine day, ten day, 11 day, 12 day, 13 day, 14 day, 15 day, 16 day, 17 day, 18 day, 19 day, 20 day, 21 day, 22 day, 23 day, 24 day, 25 day, 26 day, 27 day, 28 day, 29 day or 30 day interval. In other cases, the interval may be a range of days, for example, the range of days may be 1-5 days, 1-7 days, 1-10 days, 3-15 days, 5-10 days, 5-15 days, 5-20 days, 7-10 days, 7-15 days, 7-20 days, 7-25 days, 10-15 days, 10-20 days, 10-25 days, 15-20 days, 15-25 days, 15-30 days, 20-30 days, 20-35 days, 20-40 days, 20-50 days, 25-50 days, 30-50 days, 35-50 days, or 40-50 days.

Subjects may be evaluated after administration of the vaccine. In some cases, the subject may be evaluated within one month (e.g., short term) of the final administration of the vaccine. For example, short term may be one day, two days, three days, four days, five days, six days, seven days, eight days, nine days, ten days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or 31 days after the final administration of the vaccine. In some cases, the subject may be evaluated within four month (e.g., long term) of the final administration of the vaccine. For example, short term may be one week, two weeks, three weeks, four weeks, five weeks, six weeks, seven weeks, eight weeks, nine weeks, ten weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks or 31 weeks after the final administration of the vaccine.

In some cases, the subject may receive at least one booster dose of the vaccine after the final administration of the vaccine doses. For example, at least one booster dose may be administered to the subject one week, two weeks, three weeks, four weeks, five weeks, six weeks, seven weeks, eight weeks, nine weeks, ten weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks or 31 weeks after the final administration of the vaccine doses. In some cases, the subject may receive one booster, two boosters, three boosters, four boosters, five boosters, six boosters, seven boosters, eight boosters, nine boosters, ten boosters, 11 boosters, 12 boosters, 13 boosters, 14 boosters, 15 boosters, 16 boosters, 17 boosters, 18 boosters, 19 boosters, 20 boosters, 21 boosters, 22 boosters, 23 boosters, 24 boosters, 25 boosters, 26 boosters, 27 boosters, 28 boosters, 29 boosters or 30 booster doses.

The disclosure provides in a further aspect a pharmaceutical kit comprising an intradermal administration device and a vaccine formulation as described herein. The device is preferably supplied already filled with the vaccine. Preferably the vaccine is in a liquid volume smaller than for conventional intramuscular vaccines as described herein, particularly a volume of between about 0.05 ml and 0.2 ml. Preferably the device is a short needle delivery device for administering the vaccine to the dermis.

Suitable devices for use with the intradermal vaccines described herein include short needle devices such as those described in U.S. Pat. No. 4,886,499, U.S. Pat. No. 5,190,521, U.S. Pat. No. 5,328,483, U.S. Pat. No. 5,527,288, U.S. Pat. No. 4,270,537, U.S. Pat. No. 5,015,235, U.S. Pat. No. 5,141,496, U.S. Pat. No. 5,417,662. Intradermal vaccines may also be administered by devices which limit the effective penetration length of a needle into the skin, such as those described in WO99/34850, incorporated herein by reference, and functional equivalents thereof. Also suitable are jet injection devices which deliver liquid vaccines to the dermis via a liquid jet injector or via a needle which pierces the stratum corneum and produces a jet which reaches the dermis. Jet injection devices are described for example in U.S. Pat. No. 5,480,381, U.S. Pat. No. 5,599,302, U.S. Pat. No. 5,334,144, U.S. Pat. No. 5,993,412, U.S. Pat. No. 5,649,912, U.S. Pat. No. 5,569,189, U.S. Pat. No. 5,704,911, U.S. Pat. No. 5,383,851, U.S. Pat. No. 5,893,397, U.S. Pat. No. 5,466,220, U.S. Pat. No. 5,339,163, U.S. Pat. No. 5,312,335, U.S. Pat. No. 5,503,627, U.S. Pat. No. 5,064,413, U.S. Pat. No. 5,520,639, U.S. Pat. No. 4,596,556 U.S. Pat. No. 4,790,824, U.S. Pat. No. 4,941,880, U.S. Pat. No. 4,940,460, WO 97/37705 and WO 97/13537. Also suitable are ballistic powder/particle delivery devices which use compressed gas to accelerate vaccine in powder form through the outer layers of the skin to the dermis. Additionally, conventional syringes may be used in the classical mantoux method of intradermal administration. However, the use of conventional syringes requires highly skilled operators and thus devices which are capable of accurate delivery without a highly skilled user are preferred.

Another case of the disclosure relates to a method to immunize a subject or population of subjects against a disease in order to prevent a disease, and/or reduce the severity of disease in the subject or population of subjects. The method includes the step of administering to a subject or population of subjects that is not infected with the disease (or believed not to be infected with the disease), a composition of the disclosure.

The composition of one case of the disclosure may be administered using techniques well known to those in the art. Preferably, compounds are formulated and administered by genetic immunization. Techniques for formulation and administration may be found in “Remington's Pharmaceutical Sciences”, 18th ed, 1990, Mack Publishing Co., Easton, Pa. Suitable routes may include parenteral delivery, such as intramuscular, intradermal subcutaneous, intramedullary injections, as well as, intrathecal, direct intraventricular, intravenous, intraperitoneal, intranasal, or intraocular injections, just to name a few. Other routes include oral or transdermal delivery. For injection, the composition of one case of the disclosure may be formulated in aqueous solutions, preferably in physiologically compatible buffers such as Hanks' solution, Ringer's solution, or physiological saline buffer.

For parenteral application, which includes intramuscular, intradermal, subcutaneous, intranasal, intracapsular, intraspinal, intrasternal, and intravenous injection, particularly suitable are injectable, sterile solutions, preferably oily or aqueous solutions, as well as suspensions, emulsions, or implants, including suppositories. Formulations fix injection may be presented in unit dosage form, e.g., in ampoules or in multi-dose containers, with an added preservative. The compositions may take such forms as suspensions, solutions or emulsions in oily or aqueous vehicles, and may contain formulator agents such as suspending, stabilizing and/or dispersing agents. Alternatively, the active ingredient may be in powder form for constitution with a suitable vehicle, e.g., sterile pyrogen-free water, before use.

For enteral application, particularly suitable are tablets, dragees, liquids, drops, suppositories, or capsules. The pharmaceutical compositions may be prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. A syrup, elixir, or the like can be used wherein a sweetened vehicle is employed.

Sustained or directed release compositions can be formulated, e.g., liposomes or those wherein the active compound is protected with differentially degradable coatings, e.g., by microencapsulation, multiple coatings, etc. It is also possible to freeze dry the new compounds and use the lyophilizates obtained, for example, for the preparation of products for injection.

For administration by inhalation, the compounds for use according to one case of the present disclosure are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch. For topical, or transdermal, application, there are employed as non-sprayable forms, viscous to semi-solid or solid forms comprising a carrier compatible with topical application and having a dynamic viscosity preferably greater than water. Suitable formulations include but are not limited to solutions, suspensions, emulsions, creams, ointments, powders, liniments, salves, aerosols, etc., which are, if desired, sterilized or mixed with auxiliary agents, e.g., preservatives, stabilizers, wetting agents, buffers or salts for influencing osmotic pressure, etc. For topical application, also suitable are sprayable aerosol preparations wherein the active ingredient; preferably in combination with a solid or liquid inert carrier material, is packaged in a squeeze bottle or in admixture with a pressurized volatile, normally gaseous propellant, e.g., a freon. The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

In accordance with one case of the present disclosure the compositions may comprise a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g., intravenous, cutaneous or subcutaneous, intramucosal (e.g., gut), intranasal, intramuscular, or intraperitoneal routes.

In general the term “biologically active” indicates that a compound (including a protein or peptide) has at least one detectable activity that has an effect on the metabolic or other processes of a cell or organism, as measured or observed in vivo (i.e., in a natural physiological environment) or in vitro (i.e., under laboratory conditions).

### Immunogenicity of Compositions

The immunogenicity of the compositions described herein may be evaluated in a subject. In some cases, the epitope encoded by the composition (e.g., plasmid-based vaccines) may be evaluated in a recipient subject. For example, the recipient subject may be a rodent, a non-human primate or a human. In some cases, the rodent is a mouse. For example, the mouse may be a neu-TG mouse, a C3 mouse, or a FVB mouse.

The compositions and methods described herein may elicit an immune response to an epitope of an antigenic peptide in a subject. In some instances, the compositions may be a breast cancer vaccine. In some cases, the breast cancer vaccine may be a multiantigen breast cancer vaccine.

The immune response may be a T cell mediated response. The immune response may be a Type I immune response, a Type II immune response or both Type I and Type II immune responses. In some cases, a Type I immune response may result in the secretion of inflammatory cytokines (e.g., IFNγ, TNFα) by antigen specific T-cells. The inflammatory cytokines (e.g., Type I cytokines) may activate cytotoxic T-cells which, for example, may kill cells which express at least one epitope encoded for (e.g., nucleic acids, plasmids) or delivered (e.g., peptide, protein) by the vaccine. In some cases, the Th1 cytokines may activate additional immune cells. In some cases, a Type II immune response may result in the secretion of immunosuppressive cytokines (e.g., IL-10, IL-4 and IL-5) by regulatory T-cells. The immunosuppressive cytokines (e.g., Type II cytokines) may activate regulatory T-cells which, for example, may not kill cells which express at least one antigenic epitope encoded for (e.g., nucleic acids, plasmids) or delivered (e.g., peptide, protein) by the vaccine but rather suppress the Th1 immune response.

Whether a Th1 or a Th2 immune response, or both, may occur in a subject may be the result of the affinity between the epitope and the MHC-T cell receptor interaction. In some cases, the affinity of the binding peptides for MHC molecules may be high. In other cases, the affinity of the binding peptides for MHC molecules may be low. In some cases, low affinity binding peptides may induce a Th2 response. In other cases, high affinity binding peptides may induce a Th1 response. The affinity of candidate binding peptides for MHC molecules may be screened. For example, IFNγ and IL-10 secretion induced by a candidate binding peptide may be determined as described herein or using techniques known to one of ordinary skill in the art.

The immunogenicity of the vaccine may be analyzed in the subject using any of the plurality of methods known to one of ordinary skill in the art. In some cases, immunogenicity may be analyzed by detecting expression of peptides in the subject encoded by the vaccine administered to the subject. For example, detection methods may include ELISPOT, ELISA, Western blotting, flow cytometry, histology, chromatography, mass spectrometry and the like. Often, immunogenicity to isolated peptides produced in the subject in response to the vaccine may be analyzed. In some cases, a sample of tumor cells, cancer cells, spleen cells or normal cells taken from the subject may be analyzed.

In some cases, lymphocytes may be isolated from the subject for analysis of immunogenicity. For example, lymphocytes may be isolated from the spleen, from the lymph nodes and/or from the draining lymph nodes. In some cases, the lymphocytes may be isolated after administration of the single dose of the vaccine. In other cases, the lymphocytes may be isolated after administration of the last dose of a plurality of doses of the vaccine. For example, lymphocytes may be isolated one day, two days, three days, four days, five days, six days, seven days, eight days, nine days, ten days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days or 30 days after administration of either the single dose of the vaccine.

In some cases, the lymphocytes may be isolated after administration of the last dose of a plurality of doses of the vaccine. In other cases, the lymphocytes may be isolated after administration of the last dose of a plurality of doses of the vaccine. For example, lymphocytes may be isolated one day, two days, three days, four days, five days, six days, seven days, eight days, nine days, ten days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days or 30 days after administration of the last dose of a plurality of doses of the vaccine.

In some cases, a protein detection method may be used to determine the amount of each peptide encoded for by the nucleic acids of the composition (e.g., the plasmid-based vaccine) produced by the subject. For example, an ELISPOT may be performed and the ELISPOT may detect IFNγ. For another example, a different ELISPOT may be performed and the ELISPOT may detect Granzyme B. In some cases, a protein detection method may be used to determine the presence of protein specific T-cells in response to the composition (e.g., plasmid-based vaccine) produced by the subject. For example, an ELISPOT may be performed and the ELISPOT may detect IFNγ. For another example, a different ELISPOT may be performed and the ELISPOT may detect Granzyme B.

Immunogenicity of the peptides encoded by the vaccine may be determined by comparing the results from subjects after administration of the composition (e.g., vaccine) to the results of the methods described herein from subjects after administration of a control composition (e.g., nothing encoded by the plasmids or no peptides). In some cases, the control may be the adjuvant alone. In other cases, the control may be a negative control (e.g., blank plasmids lacking antigenic peptide epitopes). Immunogenicity may be determined by an increase in the amount of IFNγ produced (e.g., IFNγ positive spots on an ELISPOT) or increase in the amount of tumor specific Granzyme B produced (Granzyme B positive spots on an ELISPOT). The increase may be observed in subjects after administration of the composition (e.g., vaccine) compared to subjects administered a control composition. In some cases, the increase may be statistically different than the control as indicated by a P value (e.g., p<0.05). Often, statistically different at p<0.05 is statistically significant.

For example, the statistical significance of immunogenicity may be determined by comparing two groups (n=10 subjects per group) for a 98% power where at least the two-sided level may be 0.05 and the true effect size may be 2.0. In some cases, the effect size may be defined as the difference in mean specific T-cell response level divided by the common standard deviation. A true effect size of about 1.5 or less would not be significant.

Additional parameters may be analyzed after administration of at least one dose of the vaccine. In some cases, blood may be isolated from a subject and a plurality of tests performed on the blood known to one of ordinary skill in the art. For example, a basic metabolic panel and/or a complete blood count performed. In some cases, additional tissues may be examined. For example, the spleen, skin, skeletal muscle, lymph node, bone, bone marrow, ovary, oviduct, uterus, peripheral nerve, brain, heart, thymus, lung, kidney, liver and/or pancreas may be examined after administration of at least one dose of the vaccine.

### Efficacy of the Compositions Using Model Systems

The compositions described herein may be utilized with a plurality of mouse model systems. In some cases, the mouse models may include genetically diverse mouse models. In some cases, the mouse model may be a tumor implant model. For example, the mice may include, TgMMTV-neu (neu-TG) and TgC3(I)-Tag (C3). In some cases, a genetically similar mouse model may be used. For example, the neu-TG mouse model system may have a genotype similar to two different types of human cancers, (1) human luminal cancer and is estrogen receptor negative (ER−), and (2) HER2+ human breast cancer and overexpresses the neu oncogene. In other cases, the C3 mouse may have a genotype that may be similar to basal breast cancer and/or triple negative breast cancer. The mouse model of DMBA induced breast cancers in FVB mice may be heterogeneic and may have tumors comparable to multiple subtypes of human breast cancers. For example, a mouse model of genetically similar may be Medroxyprogesterone-DMBA-induced tumors in FVB mice (DMBA).

In some cases, the mouse model may be a tumor implant model. For example, a tumor implant model may be used to analyze the therapeutic efficacy of the compositions described herein. For example, the composition may be a breast cancer vaccine. In some cases, tumor cells may be implanted subcutaneously in the mouse. For example, at least 1,000, 2.500, 5,000, 7.500, 10,000, 12,500, 15,000, 17,500, 20,000, 22,500, 25,000, 27,500, 30,000, 35,000, 40,000, 45,000, 50,000, 75,000, 100,000, 125,000, 150,000, 175,000, 200,000, 225,000, 250,000, 275,000, 300,000, 350,000, 400,000, 450,000, 500,000, 750,000, 1,000,000, 1,250,000, 1,500,000, 1,750,000, 2,000,000, 2,500,000, 3,000,000, 3,500,000, 4,000,000, 4,500,000, 5,000,000, 5,500,000, 6,000,000, 6,500,000, 7,000,000, 7,500,000, 8,000,000, 8,500,000, 9,000,000, 9,500,000 or at least 1,000,000,000 tumor cells may be implanted subcutaneously in the mouse. In some cases, the tumor cells may be MMA cells.

Tumor growth may be measured using methods known to one of ordinary skill in the art. For example, methods of measurement may include tumor diameter, tumor volume, tumor mass and the like. In some cases, imaging, extraction or histologic techniques may be used. For example, any of the techniques may include use of a contrast agent.

In some cases, the efficacy of the vaccine may be determined by the size of tumor growth relative to a control (e.g., unvaccinated mouse or a mouse treated with a control vaccine). For example, in the absence of vaccination, greater than 90% of the mice may develop tumors and in the presence of vaccination, a 60% inhibition of tumor growth may be observed. In some cases, vaccination may inhibit at least 2%, 5%, 7%, 10%, 15%, 20%, 25%, 30%, 35%, 40%, 45%, 50%, 55%, 60%, 65%, 70%, 75%, 80%, 85%, 90%, 95% or at least 99% of tumor growth.

After administration of the vaccine, the subject may be 100% tumor free. In other cases, the subject may be less than 100% tumor free after administration of the vaccine. For example, the subject may be less than 99%, 95%, 90%, 85%, 80%, 75%, 70%, 65%, 60%, 55%, 50%, 45%, 40%, 35%, 30%, 25%, 20%, 15% or less than 10% tumor free after administration of the vaccine. In some cases, the subject may become tumor free hours after administration of the vaccine. For example, the subject may become tumor free one hour, two hours, three hours, four hours, five hours, six hours, seven hours, eight hours, nine hours, ten hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours or more after administration of the vaccine. In other cases, the subject may become tumor free days after administration of the vaccine. For example, the subject may become tumor free one day, two days, three days, four days, five days, six days, seven days, eight days, nine days, ten days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days or more after administration of the vaccine. In other cases, the subject may become tumor free weeks after administration of the vaccine. For example, the subject may become tumor free one week, two weeks, three weeks, four weeks, five weeks, six weeks, seven weeks, eight weeks, nine weeks, ten weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks or more after administration of the vaccine. In other cases, the subject may become tumor free months after administration of the vaccine. For example, the subject may become tumor free one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, 49 months, 50 months or more after administration of the vaccine. In other cases, the subject may become tumor free years after administration of the vaccine.

For example, the subject may become tumor free one year, two years, three years, four years, five years, six years, seven years, eight years, nine years, ten years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years or more after administration of the vaccine.

In some cases, the efficacy of the vaccine may be determined by the amount of IFNγ produced in a vaccinated subject (e.g., mouse) relative to a control (e.g., unvaccinated mouse). In some cases, the efficacy of the vaccine may be determined by the amount of IL-10 produced in a vaccinated subject (e.g., mouse) relative to a control (e.g., unvaccinated mouse).

In some aspects, polyclonality of the epitope-specific immune response may be evaluated. In some cases, an evaluation of polyclonality may be performed by assessing the production of IgG antibodies in response to epitopes of the administered vaccine. In some cases, IgGs may be elicted to one antigen. In other cases, IgGs may be elicted to multiple antigens. In some cases, a lysate may be prepared from a sample taken from a subject and evaluated from the pre-immunization and post-immunization serum of the subject. For example, a subject may be a mouse of the neu-TG mouse model and IgGs detected using a method of peptide detection, such as ELISA or ELISPOT.

In some cases, the response to each antigen between pre-vaccination subjects (e.g., mice) and post-vaccination subjects (e.g., mice) may be analyzed using statistical methods. For example, statistical methods may include analysis using single factor ANOVA. In some cases, an analysis of the number of antigens to which subjects (e.g., mice) developed immunity during the course of vaccination may be performed.

### Toxicity and Safety Profile of Compositions

The compositions described herein may be assessed for toxicity and safety. Methods to assess toxicity and safety known to one of ordinary skill in the art may be used with the compositions described herein. In some cases, a dose escalation study may be performed. In some cases, toxicity and safety studies may screen for the development of diseases in the subject, damage to organs in the subject, damage to tissues in the subject, damage to cells in the subject, blood disorders and the like. For example, diseases may include autoimmune diseases.

### Manufacture and Quality Control of Compositions

Manufacture and testing of the compositions described herein (e.g., plasmid-based vaccines) may be performed in compliance with current standards of cGMP Biologics Production Facilities (BPF). Process development may include the transfer of the candidate cells (e.g., cell line(s)) each containing the appropriate plasmid constructs with the kanamycin selection marker to the cGMP BPF. In some cases, a research bank may be generated from the bacterial stock. For example, a scaled pilot production that may match a later cGMP manufacture may be utilized to assess plasmid yield and purity. In some cases, the preliminary manufacturing batch records and quality control testing schedules may be established. For example, the master cell bank(s) may be generated from each bacterial stock. In some cases, quality control testing may be performed inclusive of; plasmid and host cell identity, plasmid copy number, purity, viability, and retention of antibiotic resistance (plasmid retention).

In some cases, finalized and approved manufacturing batch records and standard operating procedures may be followed for cGMP production and purification of the vaccine plasmid(s) and lot release criteria may be developed. In some cases, the final bulk/pooled purified product may be quality control tested in accordance with current regulatory guidelines and then may be vialed as single dose units following validated fill and finish standard operating procedures. In compliance with cGMP regulations, the vialed product may undergo quality control testing prior to final product release.

### Applications

The compositions described herein may be administered to a subject in need of a vaccine for preventing breast cancer. The methods described herein may be combined with the compositions described herein for administration to a subject in need of a vaccine for preventing breast cancer. In some cases, administration of the vaccine may initiate the elimination of cells as the cells begin to express increased levels proteins that are components of the vaccine. In some cases, the proteins may be stem cell/EMT associated. For example, increased levels of proteins may be expressed during the malignant transformation of normal cells into cancer cells, such as for example breast cancer cells. In some instances, elimination of the breast cancer before the disease becomes clinically evident may prevent the occurrence of cancer in a subject. In some cases, elimination of the breast cancer cells before the disease becomes clinically evident may prevent the occurrence of breast cancer in a subject.

The vaccine for preventing breast cancer may be administered in a single dose administered to the subject, the dose of at least 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 86 μg, 87 μg, 88 μg, 89 μg, 90 μg, 91 μg, 92 μg, 93 μg, 4 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, or at least 200 μg/plasmid. In an exemplary case, the single dose administered to the subject is 100 μg/plasmid.

The vaccine for preventing breast cancer may be administered in more than one dose administered to the subject, each dose of at least 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 86 μg, 87 μg, 88 μg, 89 μg, 90 μg, 91 μg, 92 μg, 93 μg, 4 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 114 μg, 1135 μg, 114 μg, 115 μg, 116 μg, 117 μg, 118 μg, 119 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, or at least 200 μg/plasmid. In some cases, each dose administered to the subject may be greater than or less than the previous dose administered to the subject.

The compositions described herein may be administered to a subject in need thereof of a vaccine for treating breast cancer. The methods described herein may be combined with the compositions described herein for administration to a subject in need thereof of a vaccine for treating breast cancer. In some cases, administration of the vaccine may initiate the elimination of cells that express increased levels proteins that are components of the vaccine. In some cases, the proteins may be stem cell/EMT associated. For example, increased levels of proteins may be expressed by cancer cells, such as for example breast cancer cells. In some cases, elimination of cancer cells after the disease becomes clinically evident may prevent the persistence and propagation of breast cancer in a subject. In some cases, elimination of the breast cancer cells after the disease becomes clinically evident may prevent the persistence and propagation of breast cancer in a subject.

### Subjects

The compositions described herein may be administered to a subject in need of a vaccine for breast cancer. The methods described herein may be combined with the compositions described herein for administration to a subject in need of a vaccine for breast cancer. In some cases, the vaccine may be administered to a subject who does not have breast cancer. In other cases, the vaccine may be administered to a subject who has had breast cancer. In yet other cases, the vaccine may be administered to a subject who has breast cancer.

In some cases, the subject may be a healthy individual. In some cases, the subject may be an individual with breast cancer. For example, the individual may be a patient. In some cases, the subject is a human individual. In other cases, the subject is a non-human individual. For example, non-human individuals may be a non-human primate, including such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term “subject” does not denote a particular age. Thus, both adult and newborn individuals are intended to be covered.

### Breast Cancer

Disclosed herein include a vaccine for treating a breast cancer. Also disclosed herein is a vaccine for prevention of breast cancer. Further disclosed herein is a method for reducing the tumor size of a breast cancer.

### Types of Breast Cancer

The compositions described herein may be administered to a subject in need of a vaccine for cancer, often the cancer is breast cancer. The methods described herein may be combined with the compositions described herein for administration to a subject in need of a vaccine for cancer. Often, the breast cancer may be any type of breast cancer, for example, the breast cancer may be ductal carcinoma in situ, lobular carcinoma in situ, invasive ductal carcinoma, infiltrating ductal carcinoma, inflammatory breast cancer, triple-negative breast cancer, paget disease of the nipple, phyllodes tumor, angiosarcoma, adenoid cystic carcinoma, adenocystic carcinoma, low-grade adenosquamous carcinoma, medullary carcinoma, mucinous carcinoma, colloid carcinoma, papillary carcinoma, tubular carcinoma, metaplastic carcinoma, spindle cell carcinoma, squamous carcinoma, micropapillary carcinoma and mixed carcinoma.

In some cases, the subject may be classified with a particular grade of breast cancer. For example, the grades of breast cancer may be Grade X, Grade 1, Grade 2, Grade 3 or Grade 4. For another example, breast cancers may be indicated by a category of tubule formation, nuclear grade and/or the mitotic rate. Each category may also be assigned a specific score between one and three. In some cases, the subject may have a particular stage of breast cancer. In some cases, the stages may be assigned based on the tumor, the regional lymph nodes and/or distant metastasis. For example, the stages assigned to the tumor may be TX, T0, Tis, T1, T2, T3 or T4. For example, the stages assigned to the regional lymph nodes may be NX, N0, N1, N2 or N3. For example, the stages assigned to the distant metastasis may be MX, M0 or M1. In some cases, the stages may be stage 0, stage I, stage II, stage III or stage IV. Often the breast cancer is classified as more than one grade, or stage of cancer.

### Additional Therapeutic Agents

In some instances, the breast cancer vaccine described herein is administered to a patient in combination with an additional therapeutic agent. In some instances, the additional therapeutic agent is a chemotherapeutic agent, a steroid, an immunotherapeutic agent, a targeted therapy, or a combination thereof.

In some embodiments, the additional therapeutic agent is selected from: Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriccin sodium; gemcitabine; gemcitabine hydrochloride; hydroxyurca; idarubicin hydrochloride; ifosfamide; iimofosine; interleukin II (including recombinant interleukin II, or rlL2); interferon alfa-2a; interferon alfa-2b; interferon alfa-n1; interferon alfa-n3; interferon beta-1 a; interferon gamma-1 b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

In some embodiments, the additional therapeutic agent is selected from: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen; prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriccin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-such as for example growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safiningol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

In some embodiments, the additional therapeutic agent is selected from: agents which act by arresting cells in the G2-M phases due to stabilized microtubules, e.g., Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone, Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt, Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt, Sinai School of Medicine, also known as MF-191), TMPN (Arizona State University), Vanadocene acetylacetonate, T-138026 (Tularik), Monsatrol, nanocine (also known as NSC-698666), 3-1AABE (Cytoskeleton/Mt, Sinai School of Medicine), A-204197 (Abbott), T-607 (Tuiarik, also known as T-900607), RPR-115781 (Aventis), Eleutherobins (such as Desmethyleleutherobin, Desaetyleleutherobin, soeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

In some embodiments, the additional therapeutic agent is selected from: agents that affect the tumor micro-environment such as cellular signaling network (e.g. phosphatidylinositol 3-kinase (PI3K) signaling pathway, signaling from the B-cell receptor and the IgE receptor). Examples of agents that affect the tumor micro-environment include PI3K signaling inhibitor, syc kinase inhibitor, Protein Kinase Inhibitors such as for example dasatinib, erlotinib, everolimus, gefitinib, imatinib, lapatinib, nilotinib, pazonanib, sorafenib, sunitinib, temsirolimus; Other Angiogenesis Inhibitors such as for example GT-111, JI-101, R1530; Other Kinase Inhibitors such as for example AC220, AC480, ACE-041, AMG 900, AP24534, Arry-614, AT7519, AT9283, AV-951, axitinib, AZD1152, AZD7762, AZD8055, AZD8931, bafetinib, BAY 73-4506, BGJ398, BGT226, BI 811283, B16727, BIBF 1120, BIBW 2992, BMS-690154, BMS-777607, BMS-863233, BSK-461364, CAL-101, CEP-11981, CYC116, DCC-2036, dinaciclib, dovitinib lactate, E7050, EMD 1214063, ENMD-2076, fostamatinib disodium, GSK2256098, GSK690693, INCB 18424, INNO-406, JNJ-26483327, JX-594, KX2-391, linifanib, LY2603618, MGCD265, MK-0457, MK1496, MLN8054, MLN8237, MP470, NMS-1116354, NMS-1286937, ON 01919.Na, OSI-027, OSI-930, Btk inhibitor, PF-00562271, PF-02341066, PF-03814735, PF-04217903, PF-04554878, PF-04691502, PF-3758309, PHA-739358, PLC3397, progenipoietin, R547, R763, ramucirumab, regorafenib, RO5185426, SAR103168, SCH 727965, SGI-1176, SGX523, SNS-314, TAK-593, TAK-901, TK1258, TLN-232, TTP607, XL147, XL228, XL281RO5126766, XL418, XL765.

In some embodiments, the additional therapeutic agent is selected from: inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; mTOR inhibitors; and antibodies (e.g., rituxan).

In some embodiments, the additional therapeutic agent is selected from: interferons, interleukins, Tumor Necrosis Factors, Growth Factors, or the like.

In some embodiments, the additional therapeutic agent is selected from: ancestim, filgrastim, lenograstim, molgramostim, pegfilgrastim, sargramostim; Interferons such as for example interferon alfa natural, interferon alfa-2a, interferon alfa-2b, interferon alfacon-1, interferon alfa-n1, interferon beta natural, interferon beta-1a, interferon beta-1b, interferon gamma, peginterferon alfa-2a, peginterferon alfa-2b; Interleukins such as for example aldesleukin, oprelvekin; Other Immunostimulants such as for example BCG vaccine, glatiramer acetate, histamine dihydrochloride, immunocyanin, lentinan, melanoma vaccine, mifamurtide, pegademase, pidotimod, plerixafor, poly I:C, poly ICLC, roquinimex, tasonermin, thymopentin; Immunosuppressants such as for example abatacept, abetimus, alefacept, antilymphocyte immunoglobulin (horse), antithymocyte immunoglobulin (rabbit), eculizumab, efalizumab, everolimus, gusperimus, leflunomide, muromab-CD3, mycophenolic acid, natalizumab, sirolimus; TNF alpha Inhibitors such as for example adalimumabh, afelimomab, certolizumab pegol, etanercept, golimumab, infliximab; Interleukin Inhibitors such as for example anakinra, basiliximab, canakinumab, daclizumab, mepolizumab, rilonacept, tocilizumab, ustekinumab; Calcineurin Inhibitors such as for example ciclosporin, tacrolimus; Other Immunosuppressants such as for example azathioprine, lenalidomide, methotrexate, thalidomide.

In some embodiments, the additional therapeutic agent is selected from: Adalimumab, Alemtuzumab, Basiliximab, Bevacizumab, Cetuximab, Certolizumab pegol, Daclizumab, Eculizumab, Efalizumab, Gemtuzumab, Ibritumomab tiuxetan, Infliximab, Muromonab-CD3, Natalizumab, Panitumumab, Ranibizumab, Rituximab, Tositumomab, Trastuzumab, or the like, or a combination thereof.

In some embodiments, the additional therapeutic agent is selected from: Monoclonal Antibodies such as for example alemtuzumab, bevacizumab, catumaxomab, cetuximab, edrecolomab, gemtuzumab, panitumumab, rituximab, trastuzumab; Immunosuppressants, eculizumab, efalizumab, muromab-CD3, natalizumab; TNF alpha Inhibitors such as for example adalimumab, afelimomab, certolizumab pegol, golimumab, infliximab; Interleukin Inhibitors, basiliximab, canakinumab, daclizumab, mepolizumab, tocilizumab, ustekinumab; Radiopharmaceuticals, ibritumomab tiuxetan, tositumomab; Others Monoclonal Antibodies such as for example abagovomab, adecatumumab, alemtuzumab, anti-CD30 monoclonal antibody Xmab2513, anti-MET monoclonal antibody MetMab, apolizumab, apomab, arcitumomab, basiliximab, bispecific antibody 2B1, blinatumomab, brentuximab vedotin, capromab pendetide, cixutumumab, claudiximab, conatumumab, dacetuzumab, denosumab, eculizumab, epratuzumab, epratuzumab, ertumaxomab, etaracizumab, figitumumab, fresolimumab, galiximab, ganitumab, gemtuzumab ozogamicin, glembatumumab, ibritumomab, inotuzumab ozogamicin, ipilimumab, lexatumumab, lintuzumab, lintuzumab, lucatumumab, mapatumumab, matuzumab, milatuzumab, monoclonal antibody CC49, necitumumab, nimotuzumab, oregovomab, pertuzumab, ramacurimab, ranibizumab, siplizumab, sonepcizumab, tanezumab, tositumomab, trastuzumab, tremelimumab, tucotuzumab celmoleukin, veltuzumab, visilizumab, volociximab, zalutumumab.

In some embodiments, the additional therapeutic agent is selected from: Nitrogen Mustards such as for example, bendamustine, chlorambucil, chlormethine, cyclophosphamide, ifosfamide, melphalan, prednimustine, trofosfamide; Alkyl Sulfonates like busulfan, mannosulfan, treosulfan; Ethylene Imines like carboquone, thiotepa, triaziquone; Nitrosoureas like carmustine, fotemustine, lomustine, nimustine, ranimustine, semustine, streptozocin; Epoxides such as for example, etoglucid; Other Alkylating Agents such as for example dacarbazine, mitobronitol, pipobroman, temozolomide; Folic Acid Analogues such as for example methotrexate, permetrexed, pralatrexate, raltitrexed; Purine Analogs such as for example cladribine, clofarabine, fludarabine, mercaptopurine, nelarabine, tioguanine; Pyrimidine Analogs such as for example azacitidine, capecitabine, carmofur, cytarabine, decitabine, fluorouracil, gemcitabine, tegafur; Vinca Alkaloids such as for example vinblastine, vincristine, vindesine, vinflunine, vinorelbine; Podophyllotoxin Derivatives such as for example etoposide, teniposide; Colchicine derivatives such as for example demecolcine; Taxanes such as for example docetaxel, paclitaxel, paclitaxel poliglumex; Other Plant Alkaloids and Natural Products such as for example trabectedin; Actinomycines such as for example dactinomycin; Antracyclines such as for example aclarubicin, daunorubicin, doxorubicin, epirubicin, idarubicin, mitoxantrone, pirarubicin, valrubicin, zorubincin; Other Cytotoxic Antibiotics such as for example bleomycin, ixabepilone, mitomycin, plicamycin; Platinum Compounds such as for example carboplatin, cisplatin, oxaliplatin, satraplatin; Methylhydrazines such as for example procarbazine; Sensitizers such as for example aminolevulinic acid, efaproxiral, methyl aminolevulinate, porfimer sodium, temoporfin; Protein Kinase Inhibitors such as for example dasatinib, erlotinib, everolimus, gefitinib, imatinib, lapatinib, nilotinib, pazonanib, sorafenib, sunitinib, temsirolimus; Other Antineoplastic Agents such as for example alitretinoin, altretamine, amzacrine, anagrelide, arsenic trioxide, asparaginase, bexarotene, bortezomib, celecoxib, denileukin diftitox, estramustine, hydroxycarbamide, irinotecan, lonidamine, masoprocol, miltefosein, mitoguazone, mitotane, oblimersen, pegaspargase, pentostatin, romidepsin, sitimagene ceradenovec, tiazofurine, topotecan, tretinoin, vorinostat; Estrogens such as for example diethylstilbenol, ethinylestradiol, fosfestrol, polyestradiol phosphate; Progestogens such as for example gestonorone, medroxyprogesterone, megestrol; Gonadotropin Releasing Hormone Analogs such as for example buserelin, goserelin, leuprorelin, triptorelin; Anti-Estrogens such as for example fulvestrant, tamoxifen, toremifene; Anti-Androgens such as for example bicalutamide, flutamide, nilutamide; Enzyme Inhibitors such as for example aminoglutethimide, anastrozole, exemestane, formestane, letrozole, vorozole; Other Hormone Antagonists such as for example abarelix, degarelix; Immunostimulants such as for example histamine dihydrochloride, mifamurtide, pidotimod, plerixafor, roquinimex, thymopentin; Immunosuppressants such as for example everolimus, gusperimus, leflunomide, mycophenolic acid, sirolimus; Calcineurin Inhibitors such as for example ciclosporin, tacrolimus; Other Immunosuppressants such as for example azathioprine, lenalidomide, methotrexate, thalidomide; and Radiopharmaceuticals such as for example, iobenguane.

In some embodiments, the additional therapeutic agent is selected from a checkpoint inhibitor. Exemplary checkpoint inhibitors include:

PD-L inhibitors such as Genentech's MPDL3280A (RG7446), Anti-mouse PD-L antibody Clone 10F.9G2 (Cat #BE0101) from BioXcell, anti-PD-L1 monoclonal antibody MDX-1105 (BMS-936559) and BMS-935559 from Bristol-Meyer's Squibb, MSB0010718C, mouse anti-PD-L Clone 29E.2A3, and AstraZeneca's MEDI4736:

PD-L2 inhibitors such as GlaxoSmithKline's AMP-224 (Amplimmune), and rHIgM12B7;

PD-1 inhibitors such as anti-mouse PD-1 antibody Clone J43 (Cat #BE0033-2) from BioXcell, anti-mouse PD-1 antibody Clone RMP1-14 (Cat #BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1 antibody known as ANB011, antibody MDX-1 106 (ONO-4538), Bristol-Myers Squibb's human IgG4 monoclonal antibody nivolumab (Opdivo®, BMS-936558, MDX1106). AstraZeneca's AMP-514 and AMP-224, and Pidilizumab (CT-011) from CureTech Ltd;

CTLA-4 inhibitors such as Bristol Meyers Squibb's anti-CTLA-4 antibody ipilimumab (also known as Yervoy®, MDX-010, BMS-734016 and MDX-101), anti-CTLA4 Antibody, clone 9H10 from Millipore, Pfizer's tremelimumab (CP-675,206, ticilimumab), and anti-CTLA4 antibody clone BNI3 from Abcam;

LAG3 inhibitors such as anti-Lag-3 antibody clone eBioC9B7W (C9B7W) from eBioscience, anti-Lag3 antibody LS-B2237 from LifeSpan Biosciences, IMP321 (ImmuFact) from Immutep, anti-Lag3 antibody BMS-986016, and the LAG-3 chimeric antibody A9H12;

B7-H3 inhibitors such as MGA271;

KIR inhibitors such as Lirilumab (IPH2101);

CD137 (41BB) inhibitors such as urelumab (BMS-663513, Bristol-Myers Squibb), PF-05082566 (anti-4-1BB, PF-2566, Pfizer), or XmAb-5592 (Xencor):

PS inhibitors such as Bavituximab;

and inhibitors such as an antibody or fragments (e.g., a monoclonal antibody, a human, humanized, or chimeric antibody) thereof. RNAi molecules, or small molecules to TIM3, CD52, CD30, CD20, CD33, CD27, OX40 (CD134), GITR, ICOS, BTLA (CD272), CD160, 2B4, LAIR1, TIGHT, LIGHT, DR3, CD226, CD2, or SLAM.

In some instances, the additional therapeutic agent is a CD40 agonist. The CD40 agonist can be an antibody or fragments thereof or small molecule. Exemplary CD40 agonist include: dacetuzmumab (SGN-40 or huS2C6 from Seattle Genetics), SEA-CD40 (Seattle Genetics), CP-870,893 (Pfizer), Chi Lob 7/4 (University of Southampton), or ADC-1013. Additional CD40 agonist can include those such as FGK-45 described in Medina-Echeverz et al., “Agonistic CD40 antibody induces immune-mediated liver damage and modulates tumor-induced myeloid suppressive cells” J. for ImmunoTherapy of Cancer 2(3):P174 (2014).

### Samples

A sample for analysis of the immunogenicity, safety and/or toxicity may be isolated from an individual. In some cases, the sample may be selected from the group consisting of: whole blood, fractionated blood, serum, plasma, sweat, tears, car flow, sputum, lymph, bone marrow suspension, lymph, urine, saliva, semen, vaginal flow, feces, transcervical lavage, cerebrospinal fluid, brain fluid, ascites, breast milk, vitreous humor, aqueous humor, sebum, endolymph, peritoneal fluid, pleural fluid, cerumen, epicardial fluid, and secretions of the respiratory, intestinal and genitourinary tracts. In some cases, the sample may be tissue, often a biopsy sample. For example, the biopsy may contain skin tissue, breast tissue, glandular tissue, skeletal muscle tissue and/or adipose tissue.

### Kits

Kits and articles of manufacture are also provided herein for use with one or more methods described herein. The kits can contain one or more of the polypeptides and/or one or more of the nucleic acid molecules described herein, such as the polypeptides and nucleic acid molecules identified as SEQ ID NOs: 1-12, or polypeptides and/or nucleic acid molecules having a sequence at least 40%, 50%, 60%, 70%, 80%, 90%, 95%, or more sequence homology with a polypeptide or nucleic acid molecule selected from the group consisting of SEQ ID NOs: 1-12. The kits can also contain nucleic acids that encode one or more of the polypeptides described herein. The kits can further contain adjuvants, reagents, and buffers necessary for the makeup and delivery of the vaccines.

The kits can also include a carrier, package, or container that is compartmentalized to receive one or more containers such as vials, tubes, and the like, each of the container(s) comprising one of the separate elements, such as the polypeptides and adjuvants, to be used in a method described herein. Suitable containers include, for example, bottles, vials, syringes, and test tubes. The containers can be formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions will also typically be included.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g., constant or intermittent) of a health care worker (e.g., a doctor, a registered nurse, a nurse practitioner, a physician's assistant, an orderly or a hospice worker).

### EXAMPLES

The invention is further illustrated by the following non-limiting examples.

### Example 1

### Identification of Pre-Diagnostic Tumor Antigens

This example describes the use of the TgMMTV-neu mouse mammary tumor model to identify pre-diagnostic tumor antigens and assess their function as both as a pre-diagnostic biomarker and as a vaccine to inhibit breast tumor growth.

TgMMTV-neu mice are a genetically engineered mouse model of mammary cancer which is genetically similar to human luminal breast cancer and these mice have similar endogenous immune responses and same mechanisms of immune suppression as women with breast cancer.

This study uses the TgMMTV-neu mouse mammary tumor model to identify pre-diagnostic tumor antigens and assess their function as both as a pre-diagnostic biomarker and as a vaccine to inhibit breast tumor growth.

### Methods

Mouse Serum Collection.

TgMMTV-neu mice were bred under specific pathogen free conditions. Serum samples were collected every 2 weeks by retro-orbital bleeding from 2 months old until tumor growth dictated euthanasia or until the animal was ˜1 year old. Serum samples derived from the parental FVB wild type mice were used as the control.

Serological Screening of cDNA Expression Libraries (SEREX).

A SEREX antigen screen was performed using cDNA expression library form a syngeneic tumor cell line and serum samples taken at two time points just prior to the development of palpable tumor were used for the identification of autoantibodies. A total of 3×10⁶ recombinant clones were screened for each individual. Positive clones did not react with the FVB wild-type mice. The cDNA were identified by BLAST analysis.

ELISA.

Measurement of the serum antibodies to Otud6B and Stk39 in mice were carried out by indirect ELISA with commercially available proteins. Measurement of serum antibodies to the tumor antigen Pdhx was carried out using a lysate ELISA modified to evaluate both IgM and IgG responses.

Mouse Vaccine Studies.

50 ug of the pre-diagnostic tumor antigens were used as a vaccine. Each of the antigens targets were used to vaccinate TgMMTV-neu mice with implanted tumors (3×10⁵ MMC, mouse mammary carcinoma cell line) and were compared to tumor implanted TgMMTV-neu mice vaccinated with empty vector. Vaccine efficacy is improved when the vaccine has multiple targets. Therefore, a mixture of three of the pre-diagnostic tumor targets (Pdhx, Otud6b, and Stk39) were used as a vaccine in TgMMTV-neu mice to inhibit spontaneous tumorigenesis as compared to a mixture of antigens recovered from mice who had established tumors (Arhgef2, GSN, and Swap70) and plasmid alone.

Autoantibody Detection in Pre-Diagnostic Sera in Women.

The pre-diagnostic serum samples were obtained from the Women's Health Initiative study, an observational study following 161.808 healthy post menopausal women. The samples were 188 pre-diagnostic sera, 94 from women who would eventually develop breast cancer and 94 matched controls. For the early tumor autoantibody studies 48 of the cases/controls were collected over 150 days prior to the woman developing breast cancer and 46 were collected within 150 days of the woman developing breast cancer. The expression of IgM and IgG for the early tumor targets OTUD6B, STK39, and PDHX was assessed by a customized protein microarray.

Statistical Analysis.

For all analysis, significance was assessed using a two-tailed T test or ANOVA with p<0.05 being considered significant.

### Analysis

Pre-diagnostic tumor antigen vaccines inhibits tumor growth in TgMMTV-neu mice.

Vaccinating with a panel of pre-diagnostic tumor antigens inhibits tumor growth whereas vaccinating with a panel of established tumor antigens does not inhibit tumor growth.

Autoantibodies against pre-diagnostic tumor differentiate transgenic mice that will develop mammary tumors from control mice.

Autoantibodies against these antigens are also detected in the pre-diagnostic serum of women who will develop invasive breast cancer but not in matched control women

### Example 2

### Identification and Detection of Pre-Diagnostic Tumor Antigens in Mice and Humans

This example describes the identification of pre-diagnostic tumor antigens in the TgMMTV-neu mouse and subsequent identification of transgenic mice that develop breast cancer from the control parental mice. This example also describes the demonstration that the autoantibodies to the pre-diagnostic tumor antigens are also detected in women and can identify women who would develop breast cancer from matched control women.

### Methods

Mouse Serum Collection.

TgMMTV-neu mice were bred under specific pathogen free conditions. Serum samples were collected every 2 weeks by retro-orbital bleeding from 2 months old until tumor growth dictated euthanasia or until the animal was ˜1 year old. Serum samples derived from the parental FVB wild type mice were used as the control.

Serological Screening of cDNA Expression Libraries (SEREX).

A SEREX antigen screen was performed using cDNA expression library form a syngeneic tumor cell line and serum samples taken at two time points just prior to the development of palpable tumor were used for the identification of autoantibodies. A total of 3×10⁶ recombinant clones were screened for each individual. Positive clones did not react with the FVB wild-type mice. The cDNA were identified by BLAST analysis.

ELISA.

Measurement of the serum antibodies to Otud6B, Stk39, and Lgals8 in mice were carried out by indirect ELISA with commercially available proteins. Measurement of serum antibodies to the tumor antigen Pdhx was carried out using a lysate ELISA modified to evaluate both IgM and IgG responses. Established tumor antigen ELISA as published in Lu, et al., “Evaluation of known oncoantibodies Her2, p53, and Cyclin B1 in prediagnostic breast cancer sera,” Cancer Prevention Research 5(8): 1036-1043 (2012).

Early Tumor Antigen Expression in Human Breast Cancer.

The GEO dataset GSE26304 was analyzed for expression of the genes encoding the pre-diagnostic antigens. The published gene expression data was from 31 ductal carcinoma in situ (DCIS) samples and 36 invasive ductal carcinoma (IDC) samples with 6 normal breast control. The expression of the early tumor antigens in DCIS and IDC was compared to 2 standard deviations above the mean value of expression in normal breast.

Autoantibody Detection in Pre-Diagnostic Sera in Women.

The pre-diagnostic serum samples were obtained from the Women's Health Initiative study, an observational study following 161.808 healthy post menopausal women. The samples were 188 pre-diagnostic sera, 94 from women who would eventually develop breast cancer and 94 matched controls. For the early tumor autoantibody studies 48 of the cases/controls were collected over 150 days prior to the woman developing breast cancer and 46 were collected within 150 days of the woman developing breast cancer. For the HER2, P53, and CYCB1 studies, 33 cases and 45 matched controls were used from the WHI pre-diagnostic sera. The expression of IgM and IgG for the early tumor targets OTUD6B, STK39, and PDHX was assessed by a customized protein microarray.

Tables 1(A) and 1(B) below provide a panel of “pre-diagnostic” tumor autoantibodies, both IgG and IgM, which are predictive of mice that will develop breast cancer.

TABLE 1(A)

| | | Subcellular | |
|---|---|---|---|
| Genes | Full Gene Name | Location | Homology |
| Pdhx | Pyruvate dehydrogenase complex | Mitochondria | 89% |
| Otud6b | OTU domain containing 6B | Cytoplasm and | 95% |
| | | Nucleus | |
| Stk39 | Serine/threonine kinase 39 | Cytoplasm and | 94% |
| | (STE20/SPS1 homolog, yeast) | Nucleus | |
| Zfp238 | Zinc finger protein 238 | Nucleus | 100% |
| Lgals8 | Lectin, galactoside-binging, | Cytoplasm | 80% |
| | soluble, 8 | | |
| Vps35 | Vacuolar protein sorting 35 | Cytoplasm | 99% |

TABLE 1 (B)

| | IgG | | IgM | | IgG + IgM | |
|---|---|---|---|---|---|---|
| | Pre- | | Pre- | | Pre- | |
| | diag- | Tumor | diag- | Tumor | diag- | Tumor |
| Antigens | nostic | bearing | nostic | bearing | nostic | bearing |
| Pdhx | 0.507 | 0.507 | 0.500 | 0.500 | 0.507 | 0.507 |
| Otud6b | 0.784 | 0.784 | 0.782 | 0.574 | 0.818 | 0.658 |
| Stk39 | 0.640 | 0.640 | 0.582 | 0.547 | 0.704 | 0.591 |
| Lgals8 | 0.781 | 0.813 | 0.732 | 0.574 | 0.737 | 0.593 |
| Vps35 | 0.500 | 0.500 | 0.503 | 0.500 | 0.508 | 0.500 |
| Znf238 | 0.522 | 0.548 | 0.661 | 0.541 | 0.648 | 0.534 |
| Otud6b + | 0.868 | 0.871 | 0.841 | 0.676 | 0.924 | 0.676 |
| Stk39 + | | | | | | |
| Lgals8 | | | | | | |

Table 1 (A) identifies six pre-diagnostic tumor antigens recovered from the SEREX screen of TgMMTV-neu mice. Table 1(B) identifies a panel of IgM and IgG autoantibodies to the pre-diagnostic tumor antigens comparing pre-diagnostic MMTV-neu mice verses tumor bearing mice (n=21 mice). With a panel of Otud6B, Stk39, and Lgals8 the area under the curve (AUC) was 0.924 (CI 0.81-1.0 p<0.001) with sensitivity of 0.85 and specificity of 0.9.

### Analysis

Autoantibodics against pre-diagnostic tumor antigens recovered from a SEREX screen of TgMMTV-neu mice differentiate transgenic mice that will develop mammary tumors from control mice.

Autoantibodies against these antigens are also detected in the pre-diagnostic serum of women who will develop invasive breast cancer but not in matched control women.

The panel of pre-diagnostic tumor autoantibodies have similar diagnostic utility to a previously published panel of pre-diagnostic breast cancer markers (P53, CYCB1, and HER2) (1).

There is an additive effect when the study pre-diagnostic antigens (PDHX, OTUD6B, and STK39) and the established pre-diagnostic antigens (P53, CYCB1, and HER2) panels are combined suggesting that these two panels can detect different pre-malignant sera.

Furthermore, the results are illustrated in the following figures.

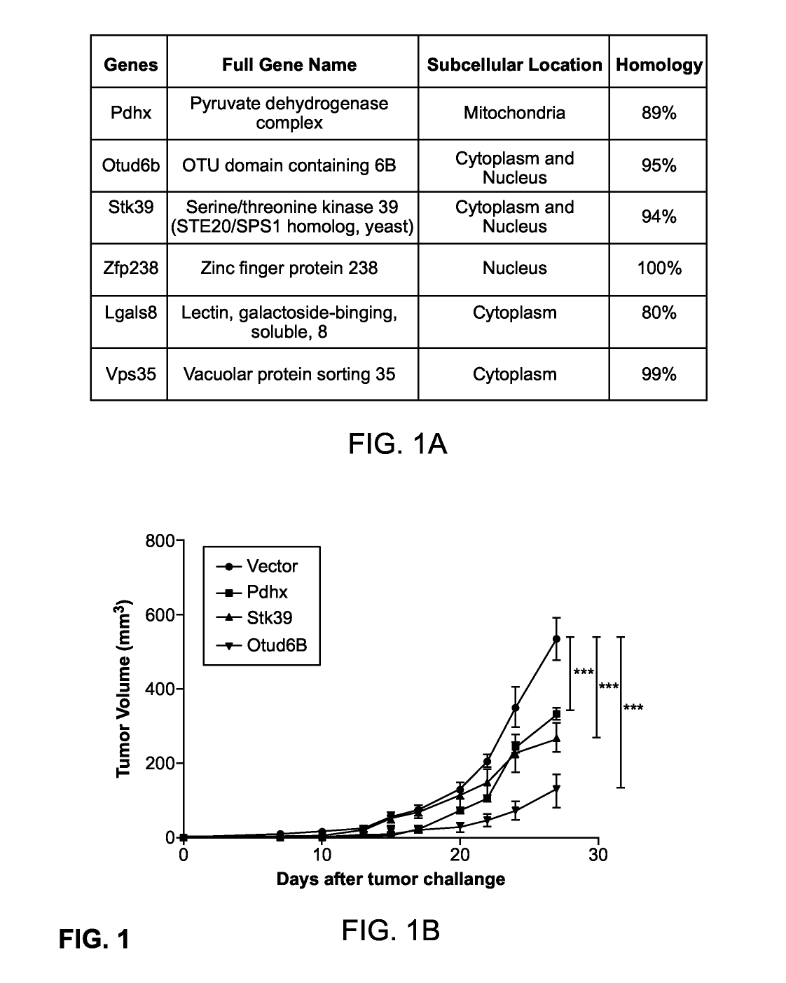

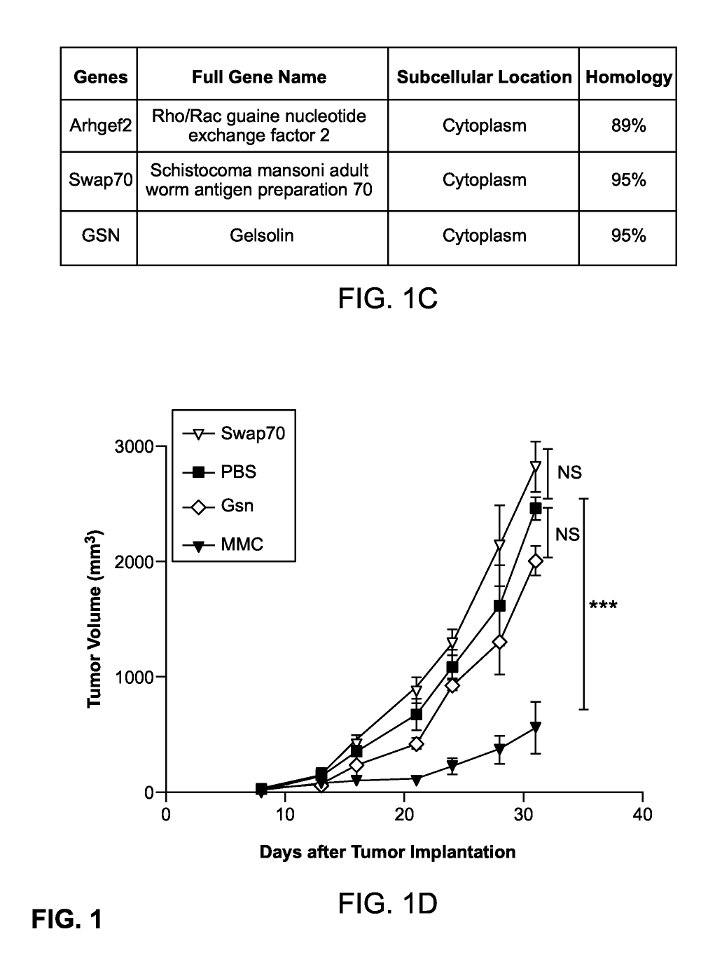

FIG. 1 shows vaccination with individual pre-diagnostic tumor antigens but not with established tumor antigens inhibits tumor growth. FIG. 1A shows six pre-diagnostic tumor antigens which are recovered from the SEREX screen of pre-diagnostic TgMMTV-neu mice. FIG. 1B shows TgMMTV-neu mice challenged with implanted 3×10⁵ MMC tumor cells after 3 vaccinations q14d with either the pre-diagnostic tumor antigens or vector control. FIG. 1C shows three established tumors recovered from SEREX screen of TgMMTV-neu mice with established tumors (adapted Lu et al Cancer Research 2006, 1). FIG. 1D shows TgMMTV-neu mice (n=3) challenged with 3×10⁵ MMC tumor cells after 3 vaccinations q14d with either the established tumor antigens, positive control irradiated MMC tumor cells, or PBS. *** p<0.001, NS no statistical difference (personal communication H. Lu).

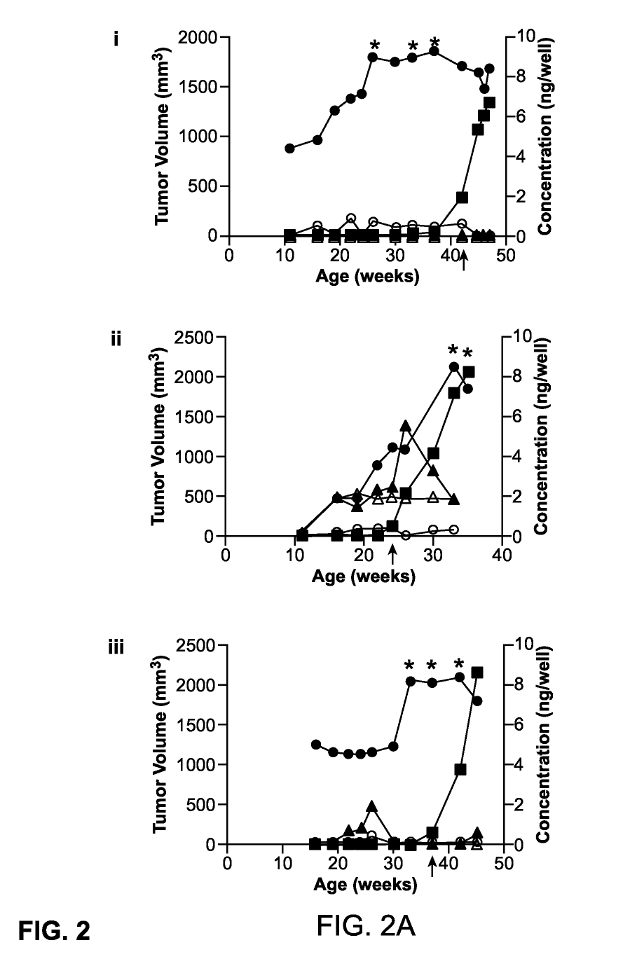

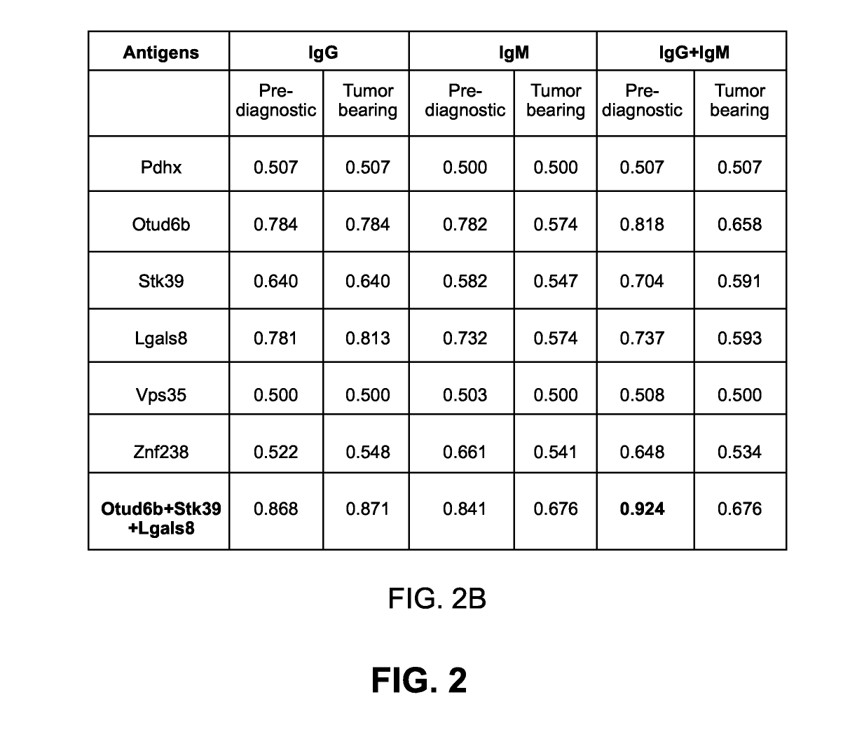

FIG. 2 illustrates pre-diagnostic tumor autoantibodies that are elevated in the serum of TgMMTV-neu mice prior to tumor development and can discriminate mice that will develop tumors. FIG. 2A shows pre-diagnostic tumor autoantibodies Pdhx (Panel i), Otud6b (Panel ii), and Stk39 (Panel iii), which are detectable in mice prior to developing palpable tumor. IgG (), IgM (⋄) antibody level and tumor growth (▪) measured in each animal with specific antibody response to antigen. IgG ( ) and IgM ( [symbol] ) measured over time from control animals. Left Y axis: Tumor volume; right Y axis: antibody titer; X axis: mouse age (in weeks). Arrow shows the time point when tumor is palpable. * indicates p<0.05 from initial value. FIG. 2B shows panel of IgM and IgG autoantibodies to the pre-diagnostic tumor antigens comparing pre-diagnostic MMTV-neu mice verses tumor bearing mice (n=21 mice). With a panel of Otud6B, Stk39, and Lgals8 the area under the curve (AUC) was 0.924 (CI 0.81-1.0 p<0.001) with sensitivity of 0.85 and specificity of 0.9.

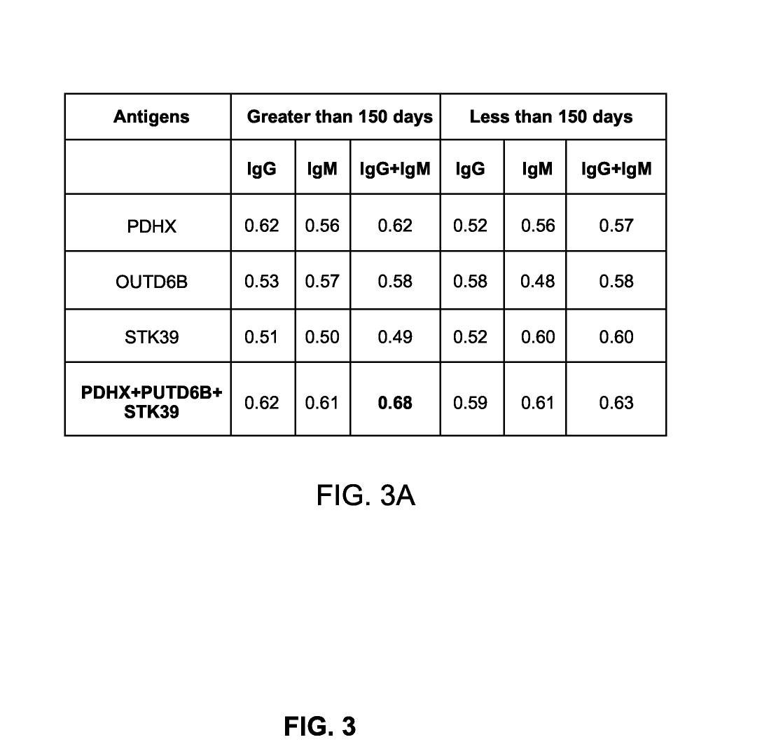

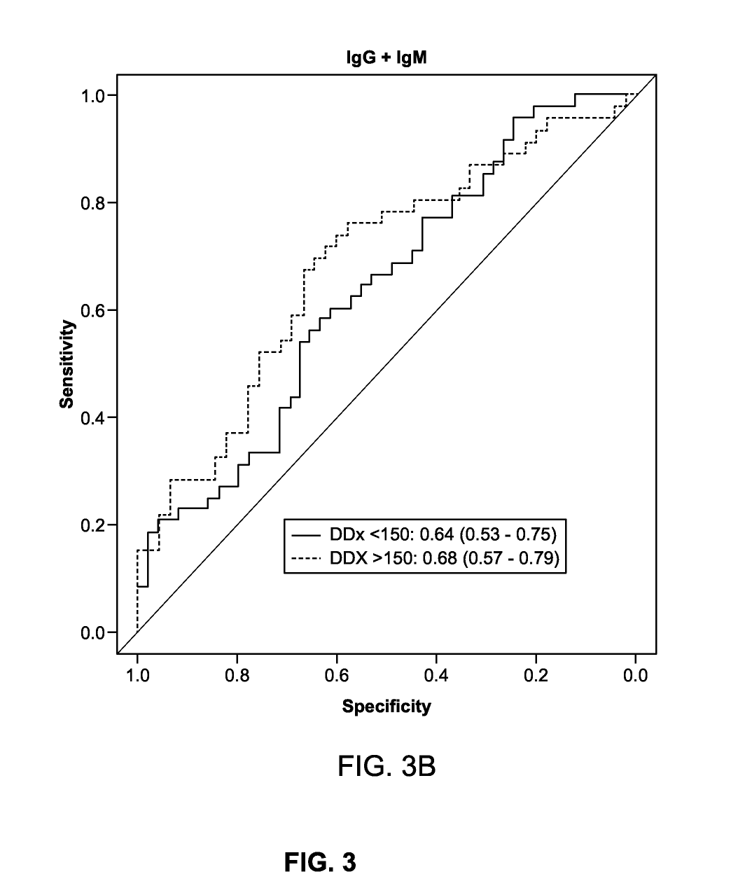

FIG. 3 shows pre-diagnostic tumor autoantibodies identified in mice that can discriminate women who will develop breast cancer from matched controls. FIG. 3A shows panel of IgM and IgG autoantibodies to early tumor antigens in pre-diagnostic sera from women who would develop breast cancer in over 150 days (n=48) verses women who would develop breast cancer within 150 days (n=46) from the WHI study. With the panel of PDHX, OTUD6B, and STK39 over 150 days prior to diagnosis of malignancy the AUC was 0.68 (CI 0.565-0.787 p=0.003) with sensitivity of 67% and specificity of 65%. FIG. 3B shows comparison of AUC of a panel of PDHX, OTUD6B, and STK39 IgG and IgM autoantibodies in women diagnosed with breast cancer more or less than 150 days prior to diagnosis.

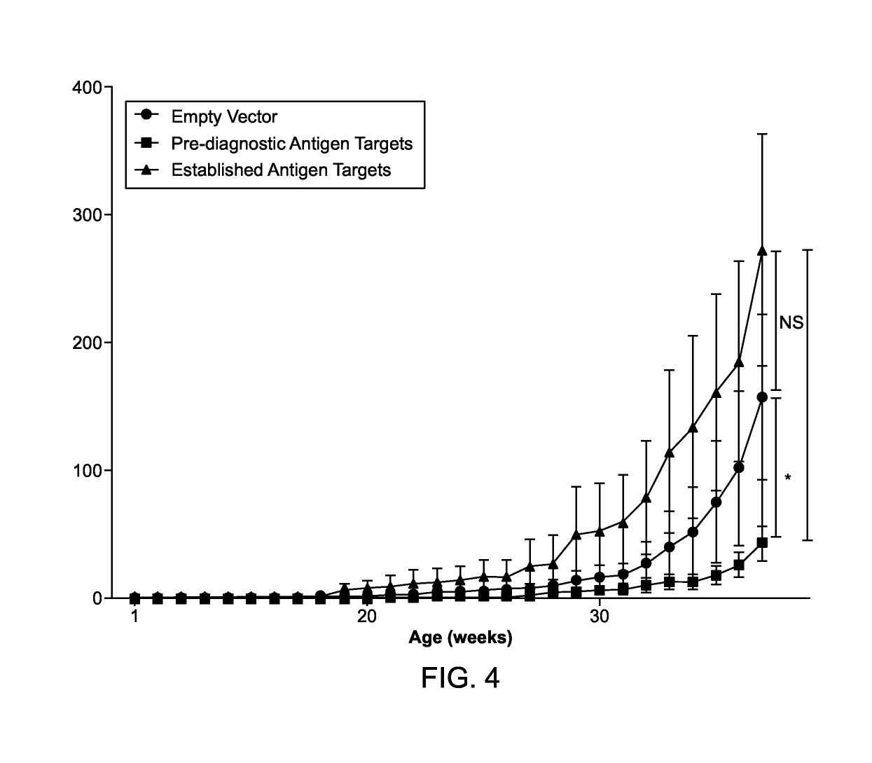

FIG. 4 shows vaccination with a panel of pre-diagnostic tumor antigens but not a panel of established tumor antigens inhibits tumor growth. Spontaneous tumor growth at 37 weeks demonstrates 34.8% decreased tumor volume as compared to vector vaccinated mice (*p=0.02). The established antigen vaccinated mice have 31.3% more growth than the pre-diagnostic antigen vaccinated mice (** p=0.005) where the growth between the established tumor and vector-vaccinated mice is not statistically different (p=0.69, NS).

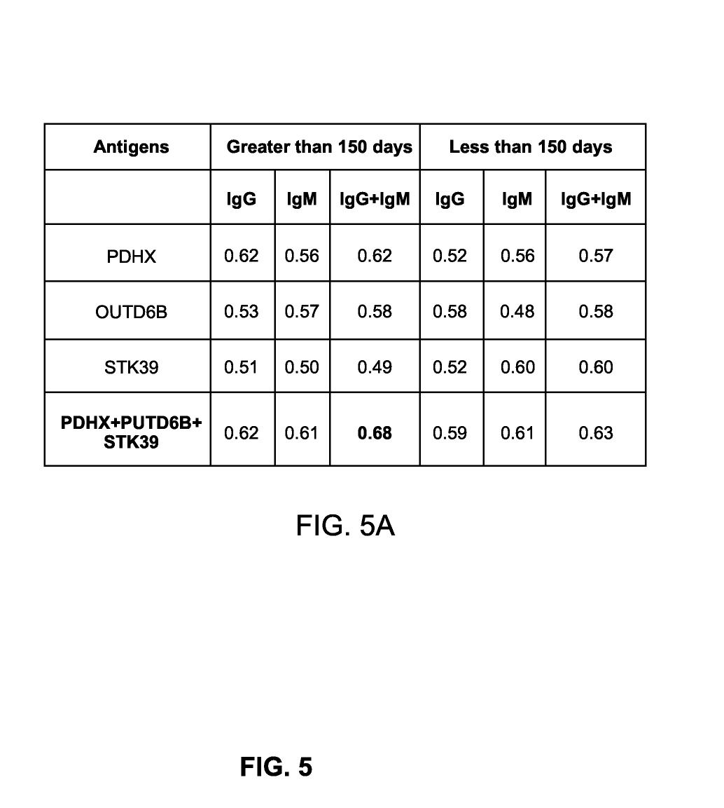

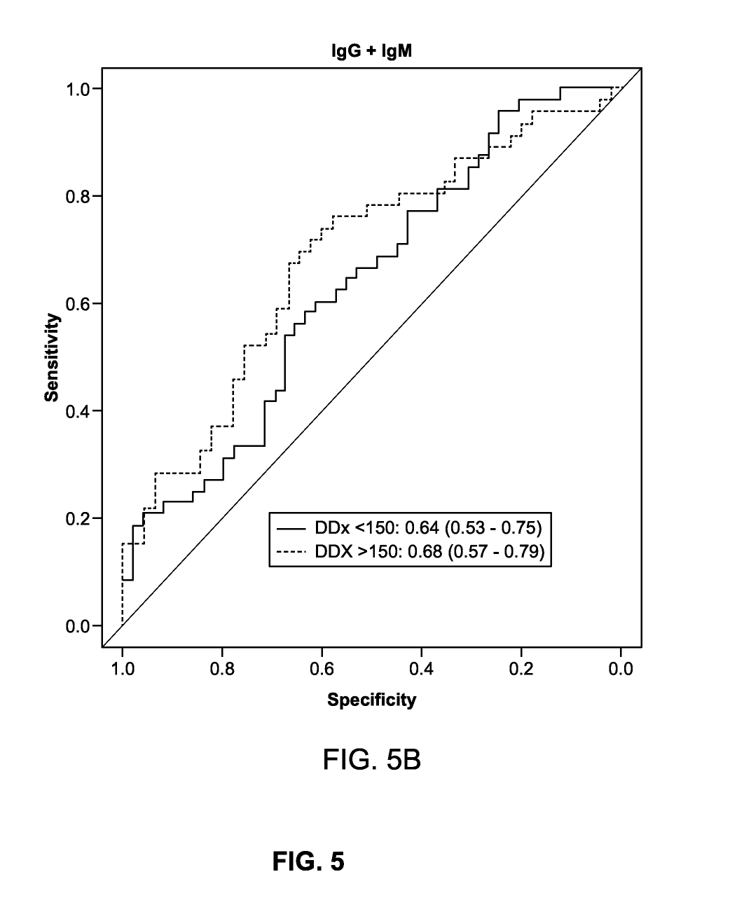

FIG. 5 shows pre-diagnostic autoantibodies identified in mice can discriminate women who will develop breast cancer from matched controls. FIG. 5A illustrates panel of IgM and IgG autoantibodies to early tumor antigens in pre-diagnostic sera from women who would develop breast cancer in over 150 days (n=48) verses women who would develop breast cancer within 150 days (n=46) from the WHI study. With the panel of PDHX, OTUD6B, and STK39 over 150 days prior to diagnosis of malignancy the AUC was 0.68 (CI 0.565-0.787 p=0.003) with sensitivity of 67% and specificity of 65%. FIG. 5B shows a comparison of AUC of a panel of PDHX, OTUD6B, and STK39 IgG and IgM autoantibodies in women diagnosed with breast cancer more or less than 150 days prior to diagnosis.

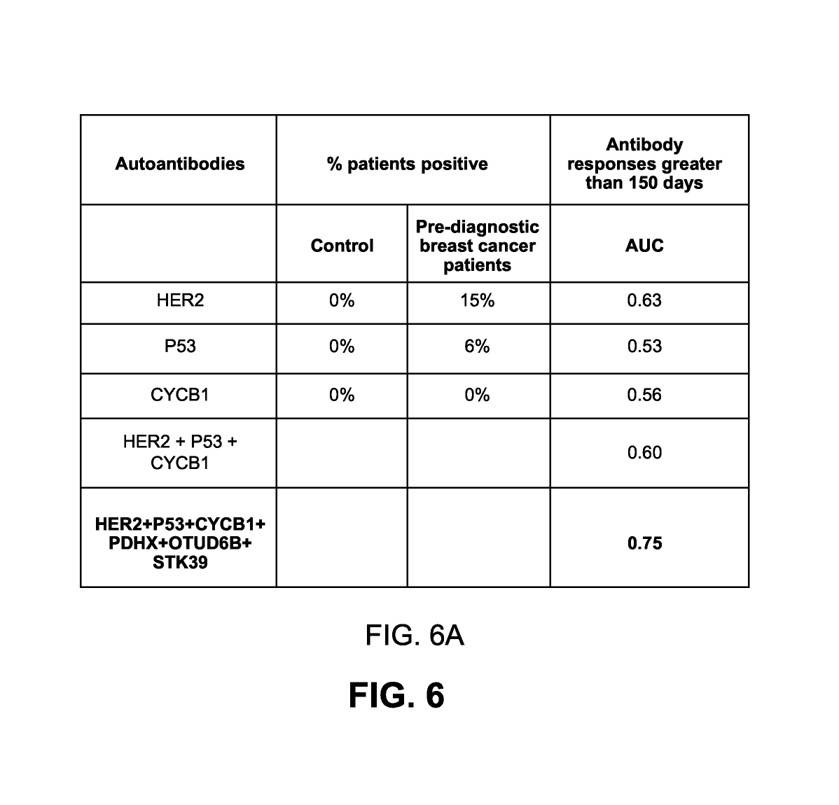

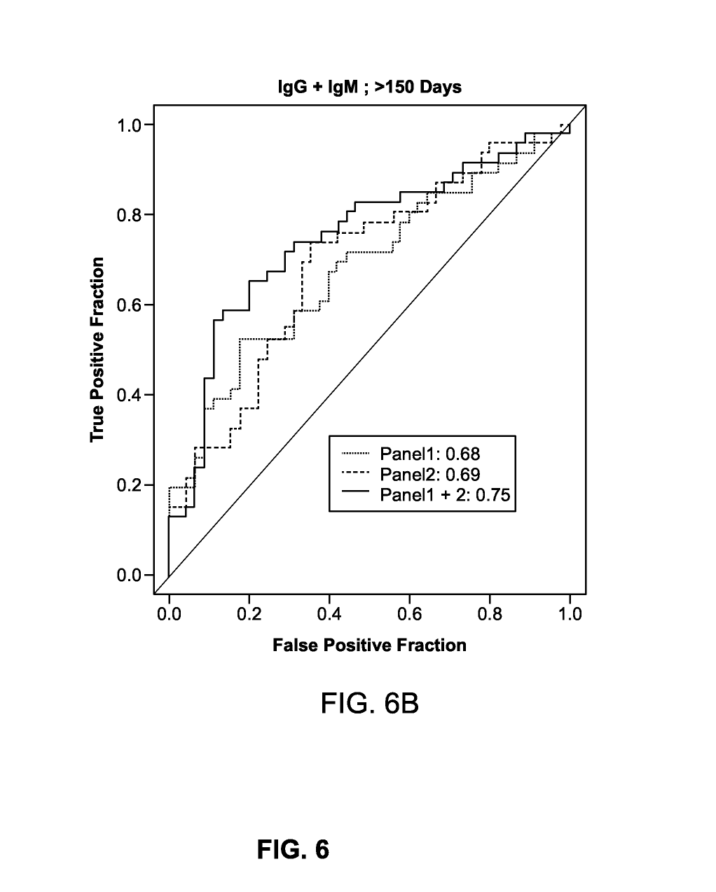

FIG. 6 shows combining pre-diagnostic autoantibodies with antibodies directed against established tumor antigens improves AUC over individual panels. FIG. 6A shows expression of established tumor antigens HER2, P53, and CYCB1 in pre-diagnostic breast cancer sera from the WHI study, figure adapted from Lu et al Cancer Prevention Research 2012 (3). FIG. 6B shows panel 1 (Pre-diagnostic tumor antigens PDHX, OTUD6B, and STK39) had similar AUC from women >150 days prior to breast diagnosis as Panel 2 (published tumor antigens HER2, p53, and Cyclin B1). When both panels are evaluated together there is an additive effect with AUC 0.75.

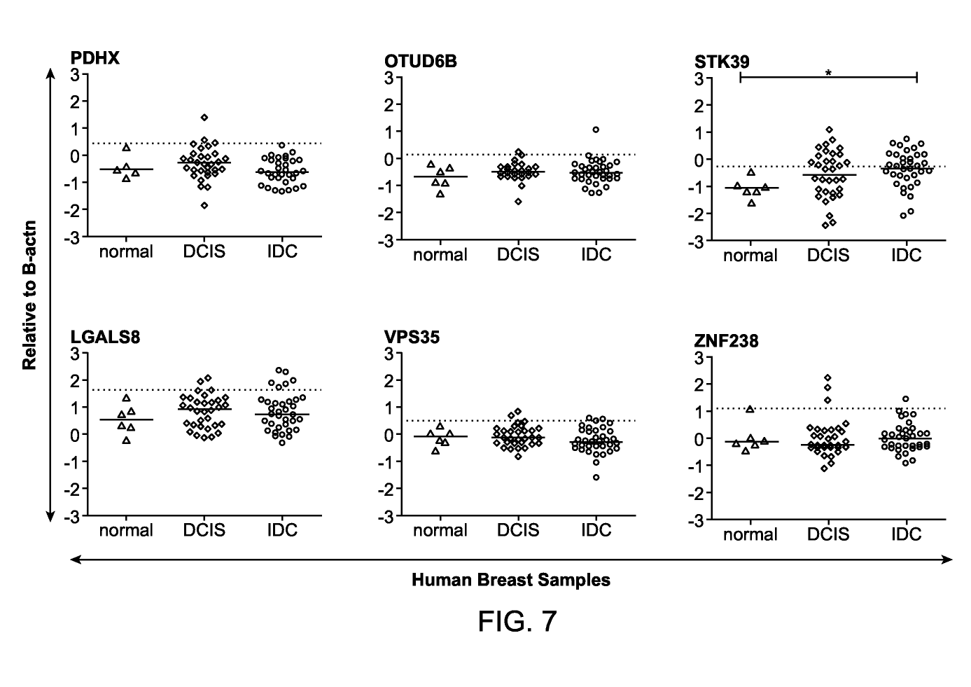

FIG. 7 shows murine pre-diagnostic antigenic proteins are expressed in human ductal carcinoma in situ and invasive breast cancer. Gene expression relative to 0 actin for the early tumor antigen proteins in normal breast (n=6), DCIS (n=31) and IDC (n=36). Dashed line: mean +2 SD above normal breast tissue. * p<0.05.

### Example 3

### Development of a Blood-Based Diagnostic Assay for Breast Cancer

This example describes the development of a blood-based diagnostic assay for breast cancer. Developing a blood-based diagnostic assay is suited to a disease such as breast cancer as high risk populations are defined and current screening tools, specifically mammography, are limited in specific populations, i.e., increased breast density in young women. Identification of autoantibodies associated with high risk or pre-invasive disease may facilitate serum-based testing for early detection or risk stratification. Unlike circulating shed tumor proteins or nucleic acids, plasma levels of which are dependent on tumor size, autoantibodies are elicited in significant concentrations even after minimal exposure to the antigen due to cytokine induced production of antibodies by activated B cells. Despite the initial promise of autoantibodies as cancer diagnostic tools, there have been few candidates that demonstrate significant predictive activity in high risk women. Progress has been challenged by a focus on single antibody markers rather than panels, antigen discovery using samples from individuals already bearing invasive cancers, and a lack of understanding of antibody kinetics developing in pre-invasive disease.

Genetically engineered mouse models of cancer may provide a model system for identification of diagnostic autoantibodies. As mice develop spontaneous breast cancers, many in middle age, serum can be collected over time and samples for discovery can span both disease-free and disease-bearing states in the same individual. The TgMMTV-neu model can be used to identify autoantibody candidates for the early detection of human breast cancer. Although the TgMMTV-neu expresses the neu proto-oncogene, the genes expressed in the tumors that arise in these animals is similar to the genes expressed in human luminal breast cancer. The mice have a latency period of pre-invasive disease, sometimes of months, before developing mammary cancers allowing the evolution of an antibody repertoire over time. The serologic screening of pre-diagnostic sera against cDNA libraries of syngeneic tumor expressed in phage to identify antibodies which were present in mice prior to the diagnosis of cancer that could discriminate at risk mice from controls were utilized. These autoantibodies were then explored in whether they had relevance in the early detection of human breast cancer using plasma samples obtained from the Women's Health Initiative (WHI) study and derived from women who would eventually develop breast cancer as compared to matched controls who remained free of disease.

### Methods

Murine Serum Samples.

TgMMTV-neu mice (strain name, FVB/N-TgN(MM TVneu)-202Mul) or wild-type FVB mice (Jackson Laboratory, San Diego, Calif.) were bred under specific pathogen-free conditions at the University of Washington. Animal care and use was in accordance with institutional and national guidelines. The protocol was approved by the University of Washington Institutional Animal Care and Use Committee (Protocol number 2878-01). Mice were enrolled for blood collection and monitoring for spontaneous tumor growth between 4-6 weeks of life. Blood samples were collected every two weeks by retro-orbital bleeding starting at the time of enrollment until tumor growth dictated euthanasia or approximately 1 year if no tumors developed. Sera was separated and stored in 10 μl aliquots at −80° C. until use. Once tumors developed, volume was measured every other day with Vernier calipers and calculated as the product of length×width×height×π/6, or the standard volume calculation for an ellipsoid shape. A colony of FVB wild-type parental animals were also bred and enrolled for blood collection between 4-6 weeks of life following the identical schema as the transgenic mice to provide control age-matched sera.

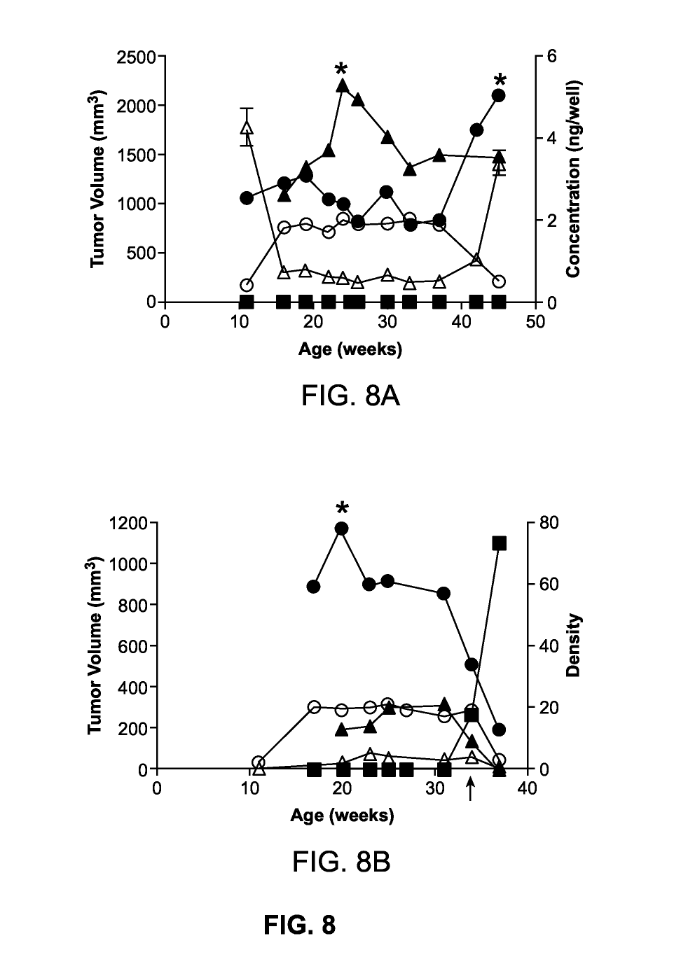

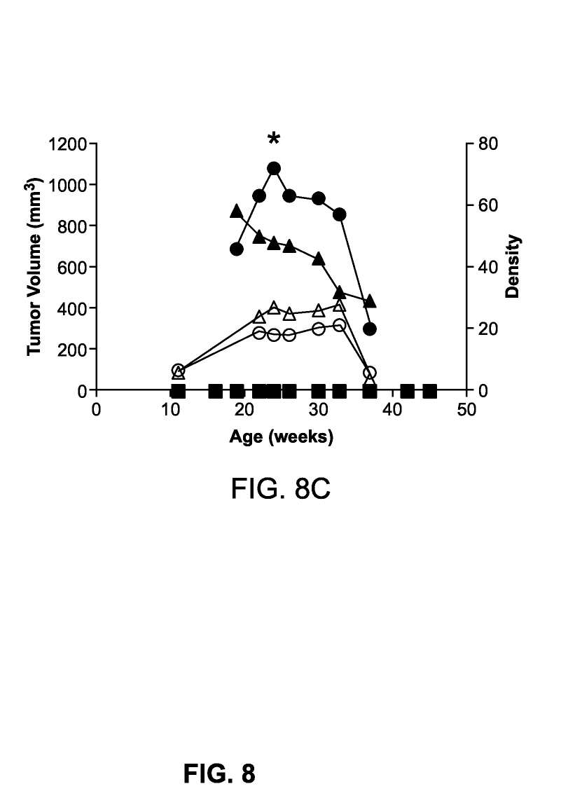

Serum, taken from animals at time-points approximately 1 month prior to the development of palpable tumor, from 20 individual TgMMTV-neu mice were used for the initial identification of antigens. i.e. pre-diagnostic sera. Pooled serum from 10 FVB wild-type mice of similar age were used as controls in initial SEREX screens as described below. Additional single individual FVB wild-type sera were used to define the kinetics of antibody responses (FIG. 2A and FIG. 8). For two of the time points, the earliest and latest in each graph. IgG and IgM levels from 20 individual FVB mice are shown (mean and SEM) (FIG. 2A and FIG. 8).

Verification of the antigens was performed using sera derived from an additional 21 TgMMTV-neu mice and an additional 26 FVB age-related wild type mice. For verification, all samples were analyzed individually. Samples from the transgenic animals were derived from two time-points in the same individual: approximately 1 month prior to the development of palpable tumors, pre-diagnostic, and the first sample time point after the development of palpable disease, tumor bearing. Similar aged samples were analyzed from the parental strain.

Human Plasma Samples.

De-identified pre-diagnostic plasma samples were collected from 188 women that participated in the WHI Observational Study. These samples consisted of 94 cases (women who eventually developed breast cancer) and 94 controls. Controls were individually matched 1:1 to cases for several variables including age at enrollment (±1 year), race, ethnicity, blood draw date, and clinical center of enrollment. The matching was also performed to ensure that each control had a similar time interval following her blood draw, as the time from blood draw to breast cancer diagnosis of the case to which she was matched. Samples were stored at −70° C. until use. Samples from 48 cases and 48 controls were collected greater than 150 days prior to the cases developing breast cancer and samples from 46 cases and 46 controls were collected within 150 days of the cases being diagnosed with breast cancer.

### Identification of Tumor Antigens

Serological screening of cDNA expression libraries (SEREX) was used to identify tumor-associated autoantibodies as previously described. Briefly, a cDNA expression library was constructed from a syngeneic tumor cell line. Pre-diagnostic serum samples pooled from 20 individual animals were used for the identification of autoantibodies. A total of 3×10⁶ recombinant clones per library were screened. A median of 4 (range 0-8) discrete positive plaques were identified from each group of 32,000 clones evaluated. The positive plaques were interrogated with pooled normal FVB serum. Clones that did not react to the FVB control serum were then purified to moncolonality and converted to pBluescript phagemid and the nucleotide sequences of the cDNA inserts were determined using an ABI Prism automated DNA sequencer. Blast was used to assess sequence homology. Six unique proteins were found to elicit autoantibodies in the pre-diagnostic sera of TgMMTV-neu mice and not the FVB parental serum pool (Table 2).

TABLE 2
Pre-diagnostic antigens identified using SEREX

| | | Subcellular | |
|---|---|---|---|
| Genes | Full Gene Name | Location | Homology |
| Pdhx | Pyruvate dehydrogenase complex | Mitochondria | 89% |
| Otud6b | OTU domain containing 6B | Cytoplasm and | 95% |
| | | Nucleus | |
| Stk39 | Serine/threonine kinase 39 | Cytoplasm and | 94% |
| | (STE20/SPS1 homolog, yeast) | Nucleus | |
| Zfp238 | Zinc finger protein 238 | Nucleus | 100% |
| Lgals8 | Lectin, galactoside-binging, | Cytoplasm | 80% |
| | soluble, 8 | | |
| Vps35 | Vacuolar protein sorting 35 | Cytoplasm | 99% |

### Evaluation of Autoantibodies in Mice.

Two methods were used for the quantitation of murine antibodies; ELISA and western blot/densitometry. The measurement of serum antibodies to tumor antigen Pdhx was carried out using a lysate ELISA (bacteria expressing the protein of interest) as previously described with the modification that both IgG and IgM responses were evaluated (Lu et al. Cancer Research 2006). Experimental serum at a 1:200 dilution and horseradish peroxidase conjugated goat anti-mouse IgG (diluted 1:5,000) or IgM (diluted 1:1000; Zymed, San Francisco, Calif.) were used. After development, plates were read at an absorbance of 450 nm. The OD of each serum dilution was calculated as the OD of the antigen-coated wells minus the OD of non-antigen encoding lysate-coated wells. The autoantibody concentration (ng/well) was calculated from the 4-parameter fitted standard curve on each plate. All identified antigens were highly homologous between mouse and man, median 95% (range 80-100). Human recombinant proteins were available for Otud6b, Stk39, and Lgals8 (all from Abnova, Walnut Calif.) and indirect ELISA was performed to measure serum antibodies to tumor antigens as described in Lu et al., “Humoral immunity directed against tumor-associated antigens as potential biomarkers for the early diagnose of cancer,” J Proteome Res 7:1388-1394 (2008). Experimental serum at a 1:200 dilution and goat anti-mouse IgG antibody (1:100,000 dilution) or goat anti-mouse IgM antibody (1:5,000 dilution) (Invitrogen, Grand Island, N.Y.) were used. Plates were read at 450 nm. The autoantibody concentration (ng/well) was calculated from the 4-parameter fitted standard curve on each plate. Five positive and negative samples were analyzed by western blot for each antigen with each assay demonstrating a sensitivity and specificity greater than 75%.

The assessment of Vps35 and Znf238 autoantibodies was performed by Western blot and densitometry as the recombinant proteins gave insufficient signal in ELISA. 3 pmole recombinant protein of Vps35 or Znf238 (Abnova) were loaded on SDS-PAGE gel and transferred onto nitrocellulose membranes (Amersham Pharmacia Biotech. Piscataway, N.J.). After blocking with 5% nonfat milk, membranes were incubated with mouse sera (diluted 1:100) overnight. Then the membranes were washed with TBS/0.05% Tween 20 and incubated with peroxidase-labeled goat anti-mouse IgG (1:1,000 dilution) or IgM (1:500 dilution) secondary antibody (Invitrogen). After washing, bands were visualized using a peroxidase-linked enhanced chemi-luminescence detection system (Amersham Pharmacia Biotech) and the optical density of the specific band was quantified with ImageJ software.

Evaluation of Antigen Expression in Human DCIS.

To evaluate whether these identified antigens had the potential for being expressed in human pre-invasive lesions, a GEO dataset, GSE26304, was analyzed for expression of the genes encoding the identified pre-diagnostic antigens. This published gene expression data was derived from 31 ductal carcinoma in situ (DCIS) and 36 invasive ductal carcinomas (IDC). Six normal breast tissue samples were included as controls. Data for the pre-diagnostic antigens are expressed as normalized to beta actin. The mean value and 2 standard deviations of the expression of a particular antigen in normal breast tissue was calculated to determine the incidence of DCIS or IDC samples having higher expression of the candidate antigens compared to normal breast tissue.

Evaluation of Pre-Diagnostic Autoantibodies in Women without Cancer

Due to the low volume of sample available for analysis, serum autoantibodies to only 3 antigens, Otud6B, Stk39, and Pdhx, were assessed using customized protein microarrays as described in Qiu et al., “Development of naturalprotein microarrays for diagnosing cancer based on an antibody response to tumor antigens,” J Proteome Res 3:261-267 (2004). These proteins were chosen as, in mice, antibodies to these 3 antigens were significantly elevated at multiple time points prior to the development of clinically palpable cancer (FIG. 2A and FIG. 8). In brief, recombinant proteins were arrayed in duplicate onto nitrocellulose-coated slides using a contact printer. Plasma samples (diluted 1:150) were hybridized with the protein microarray for 3 hours at 4° C. Slides were then incubated with Cy5-labeled anti-human IgG for 1 hour at 4° C., followed by incubation with Cy3-labeled anti-human IgM for 1 hour at 4° C. Local background-subtracted median spot intensities for downstream statistical analysis of both IgG and IgM were generated using GenePix software.

### Statistical Analysis

For all analysis, significance was assessed using a two-tailed T test or ANOVA with p<0.05 being considered significant. Differences in the incidence of an antibody response between transgenic mice and controls were evaluated using a χ² test. The sensitivity and specificity of a single or combination of antibodies in mice was evaluated using receiver-operating-characteristic (ROC) curve analyses, leading to estimates of the area under the curve (AUC), with 95% confidence intervals. Statistical analysis was carried out in SPSS software, version 15.0. For protein arrays, ROC analysis of marker combinations was performed using a linear regression model based on maximum likelihood estimation. AUC and 95% confidence intervals were calculated using R v2.13.1.

Tumor-Associated Autoantibodies were Detected in the Sera of TgMMTV-Neu Mice Prior to the Development of Palpable Disease

Pre-diagnostic autoantibodies directed against six tumor-associated antigens were identified in individual TgMM TV-neu mice at significantly higher levels compared to wild-type controls (p<0.01 for each antigen) (Table 2, FIG. 2A. FIG. 8). The antigens are all intracellular proteins of diverse functions and are involved in glycolysis, signaling, and cell adhesion pathways. Half of the antigens (Pdhx, Lgals8, and Otud6) have also been associated with inflammation and/or autoimmunity. All are highly homologous to a corresponding human protein (Table 2). Tumor growth and antibody kinetics are shown for the individual animals in which the specific antibody was detected (FIG. 2A and FIG. 8). In all individuals, antigen specific IgG antibodies significantly increased over time (p<0.05 compared to baseline) prior to tumor detection (FIG. 2A and FIG. 8). Autoantibodies specific for Lgals8 and Znf238 were discovered in mice that did not develop tumor during the time course of the study (FIG. 8, A, B). IgM antibodies were also detected prior to palpable breast tumors in most animals, in some cases at much higher levels than IgG (FIG. 2B. FIG. 8). In general, levels of IgM antibodies decreased over time, while IgG antibodies either increased or persisted at measurable levels after tumor development.

Both IgG and IgM Responses are Needed to Discriminate Serum Derived from Mice Destined to Develop Tumor as Compared to Controls

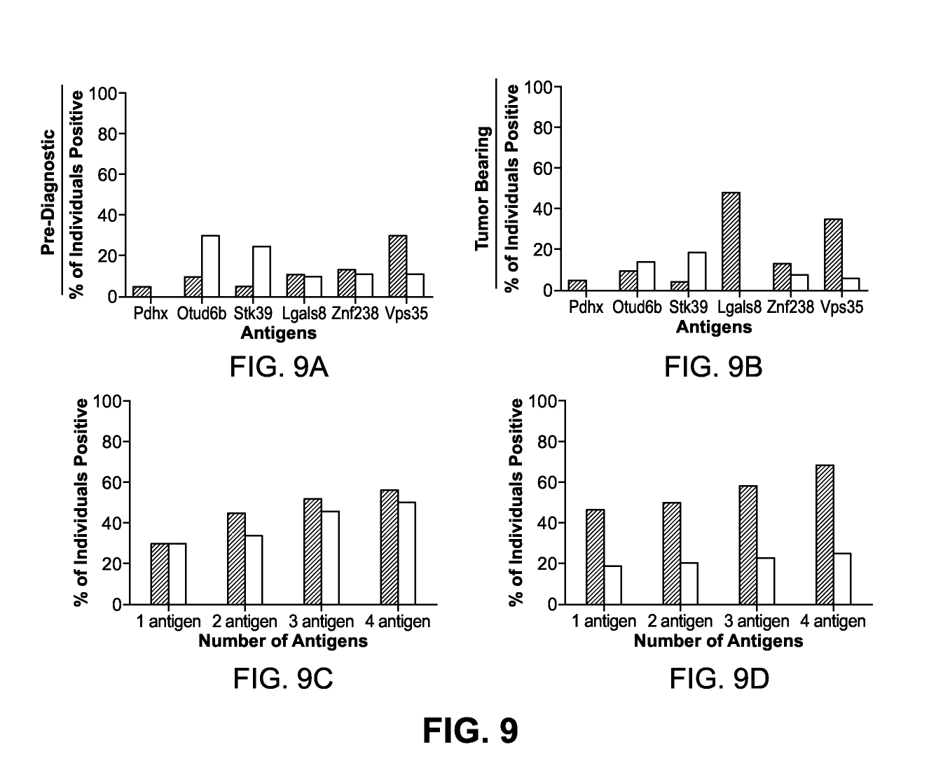

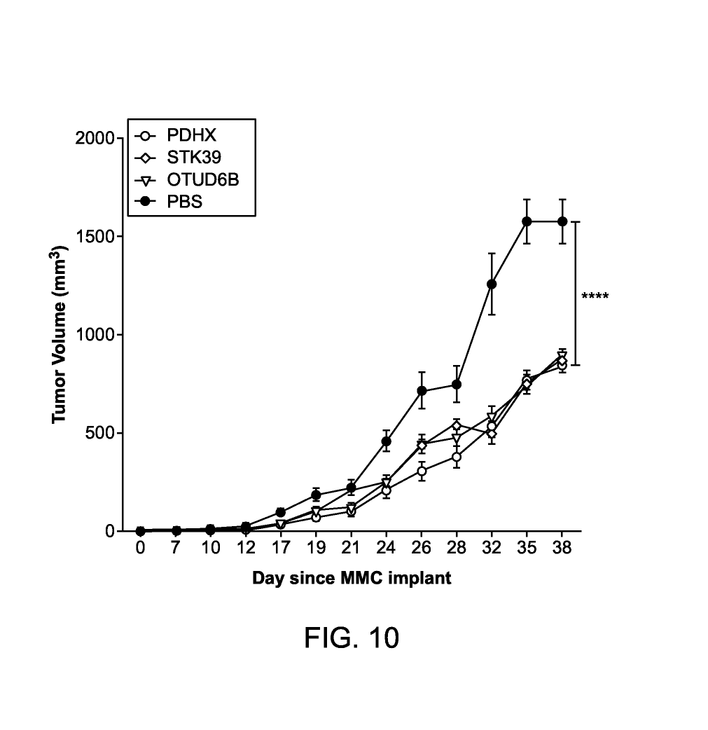

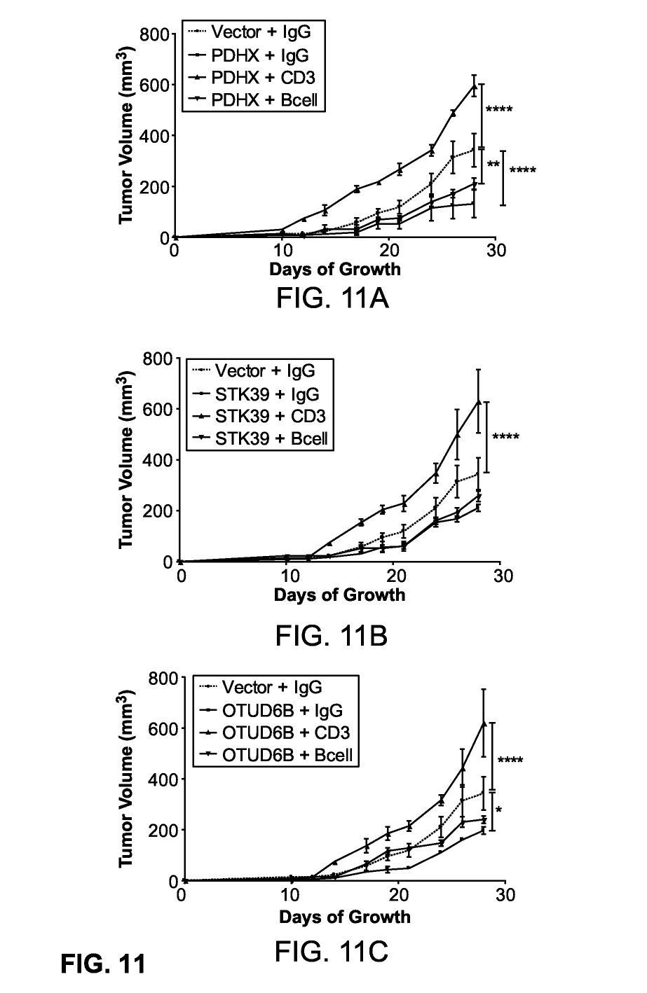

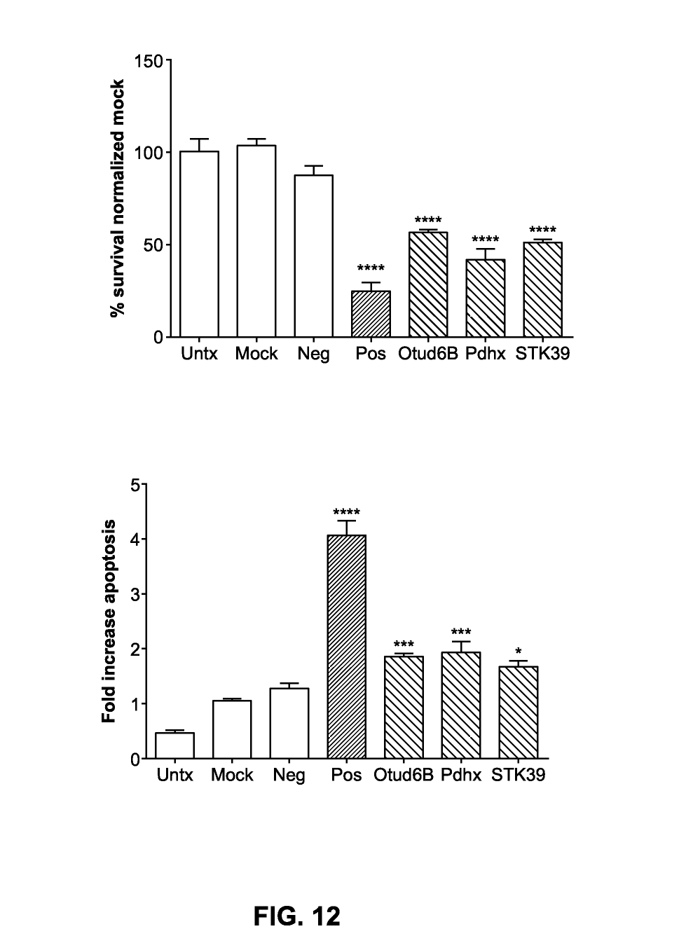

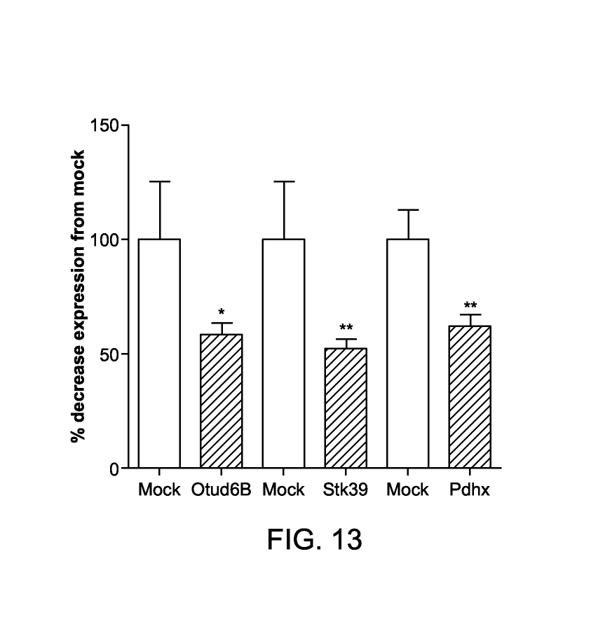

The prevalence of the pre-diagnostic autoantibodies in serum samples taken from an additional 21 transgenic animals not used for the autoantibody discovery was further evaluated. The incidence of autoantibodies to an individual antigen in mice, both pre and post tumor development, is shown in FIG. 9. The incidence of response to the antigens, prior to the development of palpable tumor, ranged from 5% (Pdhx) to 30% of mice for Vps35 for IgG. For IgM, prior to tumor detection, incidence of autoantibody responses ranged from 0% (Pdhx) to 30% (Otud6b) (FIG. 9A). In tumor bearing mice. IgG autoantibody incidence ranged from 5% (Pdhx, Stk39) to 50% (Lgals8). IgM responses were found in 0% (Pdhx, Lgals8) to 20% (Stk39) of tumor bearing mice (FIG. 9B). The incidence of IgG or IgM autoantibodies to any of the identified antigens was less than 10% of the 20 control mice, with the exception of Znf238 (22% for IgG). While IgG antibodies to any of the antigens could be detected in 68% of tumor bearing samples, IgM responses to the same proteins were present in only 25% (p<0.05) (FIG. 9D). In pre-diagnostic sera, IgM antibodies could be detected in over half the mice at levels similar to the antigen specific IgG antibodies (FIG. 9C). For a panel of all 4 markers, 56% of pre-diagnostic sera contained IgG antibodies to any of the antigens and 50% contained IgM. No individual responded to more than 4 of the antigens.

To assess the potential utility of the autoantibodies in discriminating those animals that would develop mammary tumors (pre-diagnostic and tumor bearing samples) from FVB controls, ROC were generated and AUC calculated (Table 3). The performance of IgG antibodies was equivalent in discriminating both case sample sets from controls with a panel of 3 markers demonstrating superior performance. A combination of Otud6b, Stk39 and Lgals8 gave an AUC of 0.868 (95% CI 0.744-0.968, p<0.001) for pre-diagnostic sera and 0.871 for sera de-rived from tumor bearing mice (95% CI 0.744-0.976, p<0.001) with a sensitivity of 0.75 and specificity of 0.8. The performance of the 3 antigen panel for IgM was superior in the pre-diagnostic sera with an AUC of 0.841 (95% CI 0.7-0.966, p<0.001) with a sensitivity of 0.7 and specificity of 0.9 as compared to tumor bearing sera, at 0.676 (95% CI 0.48-0.84, p=0.141). A combination of IgG and IgM was most effective in modeling early detection with an AUC of 0.924 (95% CI 0.81-1.0, p<0.001) with a sensitivity of 0.85 and specificity of 0.9 discriminating pre-diagnostic sera from FVB controls. Once animals had evidence of palpable tumor, the AUC decreased to 0.676 (95% CI 0.48-0.84, p=0.141) (Table 3).

TABLE 3
AUC of IgG and IgM antibodies against pre-diagnostic antigens

| | IgG | | IgM | | IgG + IgM | |
|---|---|---|---|---|---|---|
| | Pre- | | Pre- | | Pre- | |
| | diag- | Tumor | diag- | Tumor | diag- | Tumor |
| Antigens | nostic | bearing | nostic | bearing | nostic | bearing |
| Phdx | 0.507 | 0.507 | 0.500 | 0.500 | 0.507 | 0.507 |
| Otud6b | 0.784 | 0.784 | 0.782 | 0.574 | 0.818 | 0.658 |
| Stk39 | 0.640 | 0.640 | 0.582 | 0.547 | 0.704 | 0.591 |
| Lgals8 | 0.781 | 0.813 | 0.732 | 0.574 | 0.737 | 0.593 |
| Vps35 | 0.500 | 0.500 | 0.503 | 0.500 | 0.508 | 0.500 |
| Znf238 | 0.522 | 0.548 | 0.661 | 0.541 | 0.648 | 0.534 |
| Otud6b + | 0.868 | 0.871 | 0.841 | 0.676 | 0.924 | 0.676 |
| Stk39 + | | | | | | |
| Lgals8 | | | | | | |

### Pre-Diagnostic Tumor Antigens Identified in Mice May be Useful for the Early Detection of Human Breast Cancer

To explore the relevance of these antigens to human pre-invasive disease, we evaluated gene expression in an existing data set of normal breast, DCIS, and IDC. 10% of DCIS expressed Pdhx (FIG. 7A), Lgals8 (FIG. 7D) and Znf238 (FIG. 7F) at levels greater than 2 standard deviations above normal breast tissues, Lgals8 (FIG. 7D) was also expressed above normal levels in 17% of IDC. Stk39 (FIG. 7C) gene expression was upregulated in 50% of both DCIS and IDC compared to normal breast tissue (p<0.05). Otud6b and Vps35 expression was similar across all samples (FIG. 7B, E).

As human homologues of the murine pre-diagnostic antigens exist and the proteins appear to be expressed in human pre-invasive breast lesions, we explored the potential utility of three of the antigens, Pdhx, Otud6B, and Stk39 in discriminating women who would eventually develop cancer from controls (Table 4). Limited volumes of samples available limited our exploration to 3 of the 6 identified proteins. Otud6B and Stk39 were chosen as part of the panel of antigens that demonstrated superior performance in the mice. Pdhx was added as this antigen was identified in mice that eventually developed mammary cancer (unlike Lgals8) and Pdhx autoantibodies were significantly elevated at the earliest time points tested in the mice (FIG. 2A). An evaluation of the population as a whole revealed a range of AUC for IgG and IgM responses from 0.52-0.56 for each individual antigen. A panel of all three gave an AUC of 0.59 (95% CI 0.512-0.675, p=0.026) for IgG, 0.56 (95% CI 0.481-0.645, p=0.135) for IgM and the combination of IgG and IgM, 0.59 (95% CI 0.511-0.674, p=0.028).

Kinetic studies in mice (FIG. 2A and FIG. 8) demonstrated significant fluctuation in IgM antibody levels in time periods distant to tumor detection. Therefore, the performance of the antibody panel on the pre-diagnostic human samples separated by the case median time-from-diagnosis (150 days) were evaluated (Table 5). A linear regression analysis of IgG responses demonstrated more discrimination between case and control for samples collected greater than 150 days prior to diagnosis, AUC=0.62 (95% CI 0.506-0.735, p=0.043), than those collected closer to diagnosis, AUC=0.59 (95% CI 0.476-0.707, p=0.123). A combination of the 3 antigens for IgM responses were equivalent for samples collected both further from diagnosis, AUC=0.61 (95% CI 0.493-0.727, p=0.068), and those collected closer to diagnosis, AUC=0.61 (95% CI 0.495-0.722, p=0.064). A combination of both Ig isotypes increased the AUC of both groups, but was superior for the samples collected greater than 150 days prior to diagnosis, AUC 0.68 (CI 0.565-0.787, p=0.003). The sensitivity and specificity of the IgG+IgM panel in discriminating women greater than 150 days from their breast cancer diagnosis from control is 67% and 65% respectively. We had previously screened the WHI pre-diagnostic sera for antibodies to HER2, p53 and cyclin B1 using the same approach. Combination of IgG and IgM to all 6 antigens further increased the AUC to 0.75 in samples collected greater than 150 days from diagnosis.

TABLE 4
WHI Sample characteristics

| | | Less than 150 Days | | Greater than 150 Days | |
|---|---|---|---|---|---|
| | | Cases | Controls | Cases | Controls |
| Number | | 48 | 48 | 46 | 46 |
| Age | | 64.6 | 64.6 | 64.2 | 64.3 |
| | | (51-78) | (51-78) | 50-77) | (50-77) |
| Stage | I | — | — | — | — |
| | II | 37 | — | 35 | — |
| | III | 10 | — | 11 | — |
| | IV | 1 | — | — | — |

| | | | |
|---|---|---|---|
| Days prior to diagnosis | 82.4 | — | 209.2 |
| | (12-148) | | (151-264) |

TABLE 5
AUC of IgG and IgM antibodies against pre-diagnostic
antigens based on time to diagnosis

| | Greater than 150 days | | | Less than 150 days | | |
|---|---|---|---|---|---|---|
| Antigens | IgG | IgM | IgG + IgM | IgG | IgM | IgG + IgM |
| Pdhx | 0.62 | 0.56 | 0.62 | 0.52 | 0.56 | 0.57 |
| Otud6b | 0.53 | 0.57 | 0.58 | 0.58 | 0.48 | 0.58 |
| Stk39 | 0.51 | 0.50 | 0.49 | 0.52 | 0.60 | 0.60 |
| All 3 | 0.62 | 0.61 | 0.68 | 0.59 | 0.61 | 0.63 |

Transgenic mouse models of mammary cancer have been shown to have significant genetic similarities to human breast cancer. In addition, the serum autoantibody repertoire induced in mouse and man by breast cancer is also similar. Using SEREX, and screening tumor cDNA libraries expressed in phage with sera from cancer bearing mice, a tumor-associated autoantibody repertoire could be identified for the TgMMTV-neu. Nearly half the identified antigens were reported as human tumor antigens in a variety of cancers. Data presented here demonstrate that a pre-diagnostic autoantibody repertoire can be identified in mice prior to the development of spontaneous tumors and these antibodies may be useful for the detection of human breast cancer. Further, our work shows that detection with a combination of both IgG and IgM antibodies for a specific antigen may improve the ability to identify patients harboring the disease at time points more distant from diagnosis than is achievable by the use of IgG autoantibodies alone.

There have been several studies that have explored the use of autoantibody panels in the early detection of breast cancer. All have assessed sera from disease bearing individuals and several have focused on well-known tumor-associated antibodies such as HER2, p53, NY-ESO, and MUC1 for example. In an evaluation of 94 patients with breast cancer and 40 patients with DCIS, the percentage of patients with detectable autoantibodies specific for a panel of 7 known tumor-associated antigens, ranged from 55-73% in patients with invasive tumors but only 20-62% in women with DCIS (based on responses in volunteer controls). A variety of investigations have evaluated array-based approaches in identifying auto-antibodies that are associated with discriminating DCIS from invasive breast cancer. Almost all the auto-antibodies identified were intracellular proteins. The panels could discern DCIS from invasive cancers with AUCs of approximately 0.7-0.8 although none have been explored in high risk women. All the identified panels contained proteins that could be implicated in cancer biology and pathogenesis.

The autoantibodies found in the pre-diagnostic sera of TgMMTV-neu mice, animals that had not yet developed invasive cancers, were also directed against intracellular proteins. Half of the antigens identified are also associated with inflammation and immunity. Pdhx, a glycolysis protein, is an antigenic component of anti-mitochondrial autoantibodies. The majority of patients with primary billiary cirrhosis have autoantibodies directed against Pdhx, Lgals8 is a cytosolic lectin which has recently been shown to bind to damaged host glycans and stimulate autophagy. Antibodies to Lgals8 have been identified in a variety of autoimmune diseases including systemic lupus erythematosis and rheumatoid arthritis and may play a role in regulating autoimmune inflammation. Otud6b is a protease that cleaves ubiquitin linkages and its expression has recently been shown to be involved in the regulation of B cell proliferation after cytokine stimulation. The remaining antigens have no link to inflammation. Stk39 functions in the cellular stress pathway and activates p38 MAP kinase. Stk39 is involved in cation-chloride transport and polymorphisms in this gene are associated with the development of hypertension, Zfp238, a transcription repressor protein has been shown to be necessary for neuronal survival and development. Vps35 is part of a larger complex involved in retrograde transport of proteins from endosomes to Golgi. Mutations in Vps35 have been associated with the development of Parkinson's disease.

Inflammation has long been associated with cancer initiation due to the proliferative environment induced by innate immune cells and B cells. The presence of inflammation associated and autoimmune related antibodies in this panel may be reflective of alterations occurring at the earliest stages of the malignant transformation. However, as these auto-antibodies may also be elevated in women with chronic inflammatory and autoimmune disease, and their potential clinical utility for screening might be limited in this population.

Genes encoding homologues of murine pre-diagnostic antigens are found in human breast. DCIS, and invasive cancers. This study shows that women who eventually develop breast cancer have evidence of autoantibodies targeting these pre-diagnostic antigens prior to their first diagnosis of disease. A limited panel of 3 of the autoantibodies could discern women destined to develop breast cancer from matched controls that did not develop disease with an AUC approaching 0.7. In some cases, the use of both IgG and IgM antibodies for detection and assessing sera at a time point more distant from diagnosis enhances diagnosis. IgM antibodies are the first antibodies secreted by B cells in response to an antigen. B cell proliferation and IgM production are induced simultaneously via cytokine secretion by a number of different immune cells. As T cells become involved in antigen recognition, immunoglobulin class switching occurs with IgG responses becoming predominant and persistent and IgM antibodies wane. Indeed, several lines of evidence suggest that the presence of IgM antibodies will increase the secretion of IgG antibodies to greater levels than those achieved if IgM antibodies are not present. The kinetics of both the IgG and IgM antibody response in the TgMMTV-neu mice (FIG. 2A and FIG. 8) demonstrate that antigen specific IgM levels may be quite elevated in the time period distant from diagnosis, but decrease as IgG antibodies become elevated, similar to what is observed in viral infections. The ability to detect both IgG and IgM autoantibodies appears to provide broader population coverage in the pre-diagnostic setting in the mice and our data in women would indicate the same.

### Example 4

### Use of Otud6B, Pdhx, and Stk39 Antigens as Breast Cancer Vaccines

This example describes the determination that the Otud6B, Pdhx, and Stk39 antigens can be used as both pre-diagnostic biomarkers to predict development of breast cancer and vaccines to destroy developing breast cancer cells. Identification of proteins which are immunogenic in early breast tumor and important for breast cancer growth can allow for early breast cancer detection prior to tumor development and preventative breast cancer vaccine targets. Autoantibodies useful for breast cancer detection can identify individuals who are destined to develop breast cancer, allowing for vaccination using these early tumor antigens to prime T cells to destroy any abnormal tumor cells before they are able to establish a breast tumor. However, most tumor antigen studies have been focused on discovering immunogenic proteins from established breast tumors because there is no way to identify women before tumor development and these tumor antigens have not been effective in early detection or prevention. In this study, the TgMMTV-neu genetically engineered mouse model of breast cancer was used to discover pre-invasive tumor antigens. These mice are ideal models of human breast cancer development because they are immunocompetent and can be followed longitudinally to identify very early tumor development. Furthermore the mammary tumors in these mice are genotypically similar to human luminal breast cancer with similar tumor immune infiltrate.

The three pre-diagnostic antigens identified were Otud6B, Stk39, and Pdhx, Otud6B is a protein whose only function is in deubiquitination and which currently has no known role in cancer. Pdhx is involved in the pyruvate dehydrogenase complex and has been shown to be overexpressed in head and neck and colon cancer. Stk39 is a serine/threonine kinase and is associated with progression of non-small cell lung cancer. These proteins are homologous between mice and humans and were overexpressed in ductal carcinoma in situ and invasive breast cancer in women.

### Materials and Methods

Mouse Serum Collection.

TgMMTV-neu mice (FVB/N-Tg(MMTVneu)202Mul/J, strain #002376, Jackson Laboratories Bar Harbor, Me.) were bred under specific pathogen free conditions. The mice have a non-mutated non-activated rat neu under the control of the mouse mammary tumor virus (MMTV) promoter. All animal care and use was done in accordance with the University of Washington Institutional Animal Care and Use Committee guidelines. Serum samples were collected every 2 weeks by retro-orbital bleeding from 2 months old until tumor growth dictated euthanasia (ulceration or >1000 mm³) or until the animal was ˜1 year old. Serum samples derived from the parental FVB wild type mice were used as controls. For tumor implantation experiments, mouse mammary carcinoma cells (MMC, a syngeneic tumor cell line derived from a spontaneous tumor in a neu-tg mouse) were harvested using 2 mmol/L EDTA in PBS and washed before injection. Mice were inoculated with 3×10⁵ MMC cells subcutaneously on the mid-dorsum with a 23-gauge needle. Tumors were measured every other day with Vernier calipers and tumor volume was calculated as the product of length×width×height×r/6.

RNA Extraction and Construction of cDNA Libraries.

cDNA library was constructed as discussed previously.^{8,7} In brief, Poly(A)⁺ RNA was isolated from MMC cells using RNA4Aqeous and Poly(A)Purist kit from Ambion (Austin, Tex.). cDNA expression libraries were constructed using a ZAP Express vector from Stratagene (La Jolla, Calif.) following the manufacturer's instructions. The primary MMC library contained ˜1×10⁶ recombinants. The inserts ranged from 500 to 3,500 bp.

SEREX Screening Using Murine Sera.

A total of 1×10⁶ recombinant clones were screened with pooled sera from 20 pre-diagnostic mice. SEREX screen was performed as previously described.⁷ Briefly, 5×10³ phage clones were plated with XL-Blue on NZY agar plates. After 4 hours of incubation at 37° C. isopropyl-1-thio-β-d-galactopyranoside (IPTG)-impregnated nitrocellulose membrane was overlaid overnight on the plates to induce protein expression. The membrane was first washed in TBS with 0.05% Tween 20, blocked in TBS (20 mmol/L Tris-HCl and 150 mmol/L NaCl) with bovine serum albumin (BSA), and then incubated with 1:200 diluted sera (in TBS with 1% BSA and 0.05% sodium azide) overnight at room temperature. Alkaline phosphatase-conjugated goat anti-mouse antibody (diluted 1:2,000) was used as the secondary antibody. Nitroblue tetrazolium chloride/5-bromo-4-chloro-3-indolyl phosphate was used for color development. Positive clones that did not react to control sera from normal mice were purified to monoclonity and converted to pBluescript phagemid (pBK-CMV) by in vivo excision using XLOLR cells and ExAssist helper phage (Stratagene). Plasmid DNA was prepared using giga prep Qiagen kits (Qiagen, Valencia, Calif.), BLAST was used to search for sequence homology.

Mouse Vaccine Studies.

50 ug of the plasmids containing the portions of pre-diagnostic tumor antigens (Otud6B, Stk39, and Pdhx) recovered from the SEREX screen were used as a vaccine by intradermal injection. Negative control was empty pBK-CMV vector. Three vaccinations were performed approximately 14 days apart and for the spontaneous tumor studies booster 50 ug vaccinations of each plasmid were given monthly. For the implant studies, each of the antigens targets were used individually to vaccinate TgMMTV-neu mice and 14 days after the third vaccine, 3×10⁵ MMC were implanted subcutaneously in the mammary fat pad and were compared to tumor implanted TgMMTV-neu mice vaccinated with empty vector.

Mouse Lymphocyte Depletion Studies.

For mouse depletion studies of individual lymphocyte classes, TgMMTV-neu mice (n=3 per group) were vaccinated with plasmid DNA for the pre-diagnostic antigens Stk39, Pdhx, and Otud6B or the empty vector pBK-CMV approximately every 14 days for 3 vaccinations and then given 3×10⁵ MMC cells subcutaneously on day 0. Depletion with monoclonal antibodies was started 3 days before tumor implants with three depletions weekly the first week and then twice a week every subsequent week of CD3 (KT31.3, T cell), CD22 (CT3.4.1, B cell), or control IgG (LFT-2) (UCSF monoclonal antibody core, San Francisco Calif.).

Apoptosis and Cell Survival Assays.

Four individual siRNA for each of the pre-diagnostic and established tumor antigens were obtained from Qiagen along with the mouse All Stars positive apoptosis control, and All Stars universal negative control (Qiagen, Valencia, Calif.). For Otud6B the siRNA used were SI00819756, SI00819749, SI00819742, SI00819735, for Pdhx the siRNA used were SI01373988, SI01373981, SI01373974, SI01373967, and for Stk39 the siRNA used were SI02716189, SI01436253, SI01436246, SI01436239. 96 well black-sided sterile cell culture plates (Thermo Scientific, Waltham Mass.) were seeded with 2000 cells/well MMC cells and transfected with 40 pMol of siRNA (either 40 pMol of each siRNA for the negative and positive controls or 10 pMol of each siRNA when pooled for each of the targets) using Dharmafect liposomes as directed by the protocol (Dharmacon, Pittsburgh Pa.). Two identical plates were prepared. In one plate after 96 hours, apoptosis was measured by caspase 3/7 activation by adding 50 ul Caspase 3/7 glo reagent (Promega, Madison Wis.), incubating for 30 minutes at 37° C., and reading luciferase intensity at 90 minutes using the Wallac Envision 2104 Multi-label Detector/plate reader with a 96 well aperture (Perkin Elmer, Waltham Mass.). In the second plate after 96 hours, Cell viability was measured by ATP quantification using Cell Titer Glo (Promega, Madison Wis.) by adding 20 ul of reagent/well, incubating for 10 minutes at room temperature, and then reading luciferase at 90 minutes using the Wallac Envision 2104 Multi-label Detector/plate reader with a 96 well aperture (Perkin Elmer, Waltham Mass.). All transfections were performed in triplicate, were corrected for a no cell background luciferase control, and were normalized to averaged mock transfected wells.

mRNA Quantitation.

For quantitation of knockdown of expression for each target with siRNA, 150,000 MMC cells were seeded in 6 well plates (Corning, Corning N.Y.) and cells were collected 48 hours after transfection. Total mRNA was made using RNA-queous for PCR (Ambion, Austin Tex.), and reverse transcribed using SuperScript III First-Strand Synthesis System (Invitrogen, Grand Island N.Y.). Real-time PCR was done using an Applied Biosystems 7900. All PCR reactions used the Taqman master mix and Taqman Primer/probe sets for Otud6B, Stk39, and Pdhx were purchased from Applied Biosystems (Invitrogen, Grand Island N.Y.). mRNA expression level was normalized to mouse b-actin using the ΔC_{T} method where level of expression is equal to 2^{-ΔC}_{T}, where Δ_{CT}=C_{T} antigen −C_{T} actin. C_{T} is the cycle threshold at which the fluorescence signal crosses an arbitrary value.

Statistical Analysis

Graphs and statistical comparisons were completed using GraphPad Prism v5.03 software. A two-Way ANOVA with Bonferroni's post-test was used for grouped comparisons between three groups and a one way ANOVA with Tukey's post-test was used for comparisons between two groups. A Student's unpaired t-test was used in rtPCR siRNA knockdown analysis. Significance was considered at p<0.05 for all statistical tests.

### Use of the Pre-Diagnostic Antigens as a Biomarker of Women Who Will Develop Breast Cancer

The three pre-diagnostic antigens were tested in mouse validation cohorts to demonstrate that these autoantibodies could identify transgenic mice who would develop cancer from their parental controls. Detecting IgM and IgG antibody responses against Otud6B, Pdhx, and Stk39 antigens discriminated pre-diagnostic sera from non-transgenic control sera with an AUC of 0.924 (95% CI 0.81-1.0, p<0.001). Human samples from the Women's Health Initiative Study, an observational study following 161.808 healthy postmenopausal women to address issues of cardiovascular disease, cancer, and osteoporosis, were then examined for the presence of autoantibodies to these pre-diagnostic antigens. Samples represented 188 sera, 94 from women who would eventually develop breast cancer and 94 matched controls. IgM and IgG autoantibodies to OTUD6B, PDHX, and STK39 antigen panel could discriminate the samples of women who eventually developed breast cancer from matched controls. The discriminatory potential of the pre-diagnostic autoantibodies was enhanced if samples were collected more than 5 months prior to diagnosis (AUC 0.68; CI 0.565-0.787, p=0.003). These data suggest that the same pre-invasive breast tumor proteins are found in mice and women and can predict individuals who will subsequently develop breast cancer.

Vaccination with Pre-Diagnostic Antigens Inhibits Tumor Growth and this Tumor Inhibition is T Cell Dependent.

Using the plasmids recovered from the SEREX screen as vaccines. TgMMTV-neu mice (n=5/group) were vaccinated with the individual antigens. Vaccination with Pdhx inhibited tumor growth by 51%, Otud6B inhibited tumor growth by 53%, and Stk39 inhibited tumor growth by 51% as compared to empty vector vaccinated control at 35 weeks (p<0.0001 for each individual antigen) (FIG. 1). Furthermore, the tumor inhibition was T cell dependent because when vaccinated TgMMTV_neu mice had depletion of B cells (CD22), T cells (CD3), or a non-specific IgG antibody, only depletion of T cells reversed the inhibition of tumor growth from vaccination. In mice vaccinated with Pdhx, tumor growth was inhibited by 62% with depletion of a non-specific IgG antibody (p<0.0001) and 39% with depletion of a B cell antibody (p<0.01) as compared to empty vector control however tumor growth was increased by 73.6% (p<0.0001) as compared to empty vector control when CD3 T cells were depleted. In mice vaccinated with Stk39, tumor growth was inhibited by 38.2% with depletion of a non-specific IgG antibody and 24.4% with depletion of a B cell antibody as compared to empty vector control (p>0.05) however tumor growth was increased by 83.7% (p<0.0001) as compared to empty vector control when CD3 T cells were depleted. In mice vaccinated with Otud6B, tumor growth was inhibited by 51.1% with depletion of a non-specific IgG antibody (p<0.05) and 30% with depletion of a B cell antibody (p>0.05) as compared to empty vector control however tumor growth was increased by 80.7% (p<0.0001) as compared to empty vector control when CD3 T cells were depleted (FIG. 2). These data suggest that T cell immunity is important for tumor inhibition using these pre-diagnostic antigen vaccines.

### Pre-Diagnostic Antigens Otud6b, Pdhx, and Stk39 are Necessary for Mouse Mammary Breast Cancer Cell Survival.

Otud6b, Stk39, and Pdhx pre-diagnostic antigens were demonstrated that they were also functionally relevant for tumor growth, by showing increased apoptosis and decreased survival in syngeneic mouse mammary tumor cell lines when expression of the pre-diagnostic tumor antigens were knocked down with four pooled antigen-specific siRNA. Knocking down expression of Otud6B resulted in 44% decreased survival (p<0.0001) and 1.8 fold increased apoptosis (p<0.001) as compared to mock transfected cells. Knocking down expression of Pdhx resulted in 58% decreased survival (p<0.0001) and 1.9 fold increased apoptosis (p<0.001) as compared to mock transfected cells. Knocking down expression of Stk39 resulted in 48.7% decreased survival (p<0.0001) and 1.7 fold increased apoptosis (p<0.05) as compared to mock transfected cells. A similar significant increased apoptosis or decreased survival when using a non-targeting control was not observed (Neg, FIG. 3). Further it was confirmed that pooled siRNA targeting all tested genes resulted in significant (all p values <0.05) mRNA reduction in all cells compared to mock transected cells. Specifically, Otud6B pooled siRNA knocked down expression to 58.8% of mock (range 43.3-73.9% with each individual siRNA), Stk39 pooled siRNA knocked down expression to 52.3% of mock (range 51.8-65.8% with each individual siRNA), and Pdhx pooled siRNA knocked down expression to 61.8% of mock (range 41.2-80.8% with each individual siRNA). These data suggest that these three pre-diagnostic antigen targets (Otud6B, Stk39, and Pdhx) are functionally relevant for tumor survival and therefore are important vaccine targets for tumorigenesis.

### Example 5

### Clinical Trials

### Efficacy of a Nucleic Acid Vaccine

The purpose of the clinical trial is to determine the efficacy of a nucleic acid vaccine described herein. Women diagnosed with pre-invasive ductal carcinoma in situ (DCIS) are screened to determine if they have autoantibodies to OTUD6B, STK39, and PDHX. Women who are tested positive will be vaccinated with three vaccines prior to surgery and then at surgery the pathology specimen will be compared with a control group of women who have not been vaccinated. The pathology specimens will be evaluated to determine whether the DCIS has been eliminated by vaccination, the expression level of OTUD6B, STK39, and PDHX in the remaining DCIS, and whether the tumor immune environment has been changed to a more Th1 immune environment by vaccination.

### Long Term Study of a Nucleic Acid Vaccine

The clinical trial will first identify women who are at risk for developing breast cancer by detecting the presence or absence and/or the concentration level of the autoantibodies OTUD6B, STK39, and PDHX and then vaccinate those who are at risk to prevent breast cancer development. The trial will involve screening one to five thousand women for the autoantibodies, identify women whom are at risk for developing breast cancer (i.e., women who are positive for the presence and/or elevated concentration of the autoantibodies), and women who are positive will then be vaccinated with a nucleic acid (e.g., DNA) vaccine described here. After vaccination, the women will be followed for 10 years. A control group will also be evaluated concurrently. The control group includes women who have screened positive but have not received vaccination. Comparison of the control group with the vaccinated group will be carried out to determine if there are differences in the rate of breast cancer development (e.g., invasive breast cancer or pre-invasive ductal carcinoma in situ (DCIS)).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

TABLE 6
Antigen sequences as described herein.

| Name | Sequence | SEQ ID NO: |
|---|---|---|
| Otud6B | MEAVLTEELDEEEQLLRRHRKEKKELQAKIQGMKNAVPKNDK | 1 |
| (human) | KRRKQLTEDVAKLEKEMEQKHREELEQLKLTTKENKIDSVAVN | |
| AAH29760 | ISNLVLENQPPRISKAQKRREKKAALEKEREERIAEAEIENLTGA | |
| GI: 20987239 | RHMESEKLAQILAARQLEIKQIPSDGHCMYKAIEDQLKEKDCAL | |
| | TVVALRSQTAEYMQSHVEDFLPFLTNPNTGDMYTPEEFQKYCE | |
| | DIVNTAAWGGQLELRALSHILQTPIEIIQADSPPIIVGEEYSKKPLI | |
| | LVYMRHAYGLGEHYNSVTRLVNIVTENCS | |
| Otud6B | MEEVVAEELDDEEQLVRRHRKEKKELQAKIQGMKNAVPKNDK | 2 |
| (Mouse) | KRRKQLTEDVAKLEREMEQKHREELEQLKQLTFKDSKIDSVAV | |
| AAH87552 | NISNLVLENQPPRISKAQKRREKKAALEKEREERIAEAEIENLSG | |
| GI: 56541230 | ARHLESEKLAQILAARELEIKQIPSDGHCMYGALEDQLREQDCA | |
| | LTVASLRRQTAEYMQTHSDDFLPFLTNPSTGDMYTPEEFGKYC | |
| | DDIVNTAAWGGQLELRALSHILQTPIEILQADAPPIIVGEEYPRNP | |
| | LVLVYMRHAYGLGEHYNSVTRLVNSATENCS | |
| Pdhx | MAASWRLGCDPRLLRYLVGFPGRRSVGLVKGALGWSVSRGAN | 3 |
| (Human) | WRWFHSTQWLRGDPIKILMPSLSPTMEEGNIVKWLKKEGEAVS | |
| AAH10389 | AGDALCEIETDKAVVTLDASDDGILAKIVVEEGSKNIRLGSLIGL | |
| GI: 14714514 | IVEEGEDWKHVEIPKDVGPPPPVSKPSEPRPSPEPQISIPVKKEHIP | |
| | GTLRFRLSPAARNILEKHSLDASQGTATGPRGIFTKEDALKLVQ | |
| | LKQTGKITESRPTPAPTATPTAPSPLQATAGPSYPRPVIPPVSTPG | |
| | QPNAVGTFTEIPASNIRRVIAKRLTESKSTVPHAYATADCDLGA | |
| | VLKVRQDLVKDDIKVSVNDFIIKAAAVTLKQMPDVNVSWDGE | |
| | GPKQLPFIDISVAVATVKGLLTPIIKDAAAKGIQEIADSVKALSK | |
| | KARDGKLLPEEYQGGSFSISNLGMFGIDEFTAVINPPQACILAVG | |
| | RFRPVLKLTEDEEGNAKLQQRQLITVTMSSDSRVVDDELATRFL | |
| | KSFKANLENPIRLA | |
| Pdhx | MAASWRLHCNQPLLRYLLGFSSRRSLGLAQGAAAWPVDRGAS | 4 |
| (Mouse) | WRWFHSTQLLQADPIKVLMPSLSPTMEQGNIVKWLRKEGEAVS | |
| EDL27682 | AGDSLCEIETDKAVVTLDANDDGILAKIVVEEGAKNIQLGSLIAL | |
| GI: 148695735 | MVEEGEDWKQVEIPKDVSAPPPVSKPPAPTQPSPQPQIPCPARKE | |
| | HKGTARFRLSPAARNILEKHSLDASQGTATGPRGIFTKEDALKL | |
| | VELKQMGKITESRPASAPPPSLSASVPPQATAGPSYPRPMTPPVS | |
| | IPGQPNAAGTFTEIPASNIRRVIAKRLTESKSTVPHAYATADCDL | |
| | GAVLKVRRDLVKDDIKVSVNDFIIRAAAVTLKQMPGVNVTWD | |
| | GEGPKQLPSVDISVAVATDKGLITPIIKDAAAKGIQEIADSVKVL | |
| | SKKARDGKLMPEEYQGGSFSISNLGMFGIDEFAAVINPPQACILA | |
| | VGRFRPVLKLTEDEEGNPQLQQHQLITVTMSSDSRVVDDELAT | |
| | RFLETFKANLENPMRLG | |
| Stk39 | MAEPSGSPVHVQLPQQAAPVTAAAAAAPAAATAAPAPAAPAA | 5 |
| (Human) | PAPAPAPAAQAVGWPICRDAYELQEVIGSGATAVVQAALCKPR | |
| EAX11304 | QERVAIKRINLEKCQTSMDELLKEIQAMSQCSHPNVVTYYTSFV | |
| GI: 119631709 | VKDELWLVMKLLSGGSMLDIIKYIVNRGEHKNGVLEEAIIATIL | |
| | KEVLEGLDYLHRNGQIHRDLKAGNILLGEDGSVQIADFGVSAFL | |
| | ATGGDVTRNKVRKTFVGTPCWMAPEVMEQVRGYDFKADMWS | |
| | FGITAIELATGAAPYHKYPPMKVLMLTLQNDPPTLETGVEDKE | |
| | MMKKYGKSFRKLLSLCLQKDPSKRPTAAELLKCKFFQKAKNRE | |
| | YLIEKLLTRTPDIAQRAKKVRRVPGSSGHLHKTEDGDWEWSDD | |
| | EMDEKSEEGKAAFSQEKSRRVKEENPEIAVSASTIPEQIQSLSVH | |
| | DSQGPPNANEDYREASSCAVNLVLRLRNSRKELNDIRFEFTPGR | |
| | DTADGVSQELFSAGLVDGHDVVIVAANLQKIVDDPKALKTLTF | |
| | KLASGCDGSEIPDEVKLIGFAQLSVS | |
| Stk39 | MAEPSGSPVHVQLSQQAAPVTAAAATAPAAATSAPAPAPAPAP | 6 |
| (Mouse) | AASAAPAPAPAAAPAPAPAAQAVGWPICRDAYELQEVIGSGAT | |
| AAH64443 | AVVQAALCKPRQERVAIKRINLEKCQTSMDELLKEIQAMSQCS | |
| GI: 39963615 | HPNVVTYYTSFVVKDELWLVMKLLSGGSMLDIIKYIVNRGEHK | |
| | NGVLEEAIIATILKEVLEGLDYLHRNGQIHRDLKAGNILLGEDGS | |
| | VQIADFGVSAFLATGGDVTRNKVRKTFVGTPCWMAPEVMEQV | |
| | RGYDFKADMWSFGITAIELATGAAPYHKYPPMKVLMLTLQND | |
| | PPTLETGVEDKEMMKKYGKSFRKLLSLCLQKDPSKRPTAAELL | |
| | KCKFFQKAKNREYLIEKLLTRTPDIAQRAKKVRRVPGSSGHLHK | |
| | TEDGDWEWSDDEMDEKSEEGKAAASQEKSRRVKEENSEISVN | |
| | AGGIPEQIQSLSVHDSQAQPNANEDYREGPCAVNLVLRLRNSRK | |
| | ELNDIRFEFTPGRDTADGVSQELFSAGLVDGHDVVIVAANLQKI | |
| | VDDPKALKTLTFKLASGCDGSEIPDEVKLIGFAQLSVS | |
| Zfp238 | MEFPDHSRHLLQCLSEQRHQGFLCDCTVLVGDAQFRAHRAVV | 7 |
| (Mouse) | ASCSMYFHLFYKDQLDKRDIVHLNSDIVTAPAFALLLEFMYEG | |
| AAH54742.1 | KLQFKDLPIEDVLAAASYLHMYDIVKVCKKKLKEKATTEADST | |
| GI: 32451724 | KKEEDASSCSDKVESLSDGSSHMAGDLPSDEDEGEDDKLNILPS | |
| | KRDLAAEPGNMWMRLPSDSAGIPQAGGEAEPHATAAGKTVAS | |
| | PCSSTESLSQRSVTSVRDSADVDCVLDLSVKSSLSGVENLNSSYF | |
| | SSQDVLRSNLVQVKVEKEASCDESDVGTNDYDMEHSTVKESVS | |
| | TNNRVQYEPAHLAPLREDSVLRELDREDKASDDEMMTPESERV | |
| | QVEGGMENSLLPYVSNILSPAGQIFMCPLCNKVFPSPHILQIHLS | |
| | THFREQDGIRSKPAADVNVPTCSLCGKTFSCMYTLKRHERTHSG | |
| | EKPYTCTQCGKSFQYSHNLSRHAVVHTREKPHACKWCERRFTQ | |
| | SGDLYRHIRKFHCELVNSLSVKSEALSLPTVRDWTLEDSSQELWK | |
| Zfp238 | MCPKGYEDSMEFPDHSRHLLQCLSEQRHQGFLCDCTVLVGDA | 8 |
| (Human) | QFRAHRAVLASCSMYFHIFYKDQLDKRDIVHLNSDIVTAPAFAL | |
| AAH36677.2 | LLEFMYEGKLQFKDLPIEDVLAAASYLHMYDIVKVCKKKLKEK | |
| GI: 54311160 | ATTEADSTKKEEDASSCSDKVESLSDGSSHIAGDLPSDEDEGED | |
| | EKLNILPSKRDLAAEPGNMWMRLPSDSAGIPQAGGEAEPHATA | |
| | AGKTVASPCSSTESLSQRSVTSVRDSADVDCVLDLSVKSSLSGV | |
| | ENLNSSYFSSQDVLRSNLVQVKVEKEASCDESDVGTNDYDMEH | |
| | STVKESVSTNNRVQYEPAHLAPLREDSVLRELDREDKASDDEM | |
| | MTPESERVQVEGGMESSLLPYVSNILSPAGQIFMCPLCNKVFPSP | |
| | HILQIHLSTHFREQDGIRSKPAADVNVPTCSLCGKTFSCMYTLKR | |
| | HERTHSGEKPYTCTQCGKSFQYSHNLSRHAVVHTREKPHACK | |
| | WCERRFTQSGDLYRHIRKFHCELVNSLSVKSEALSLPTVRDWTL | |
| | EDSSQELWK | |
| Lgals8 | MLSLNNLQNIIYNPIIPYVGTITEQLKPGSLIVIRGHVPKDSERFQ | 9 |
| (Mouse) | VDFQLGNSLKPRADVAFHFNPRFKRSSCIVCNTLTQEKWGWEEI | |
| CAJ18459.1 | TYDMPFRKEKSFEIVFMVLKNKFQVAVNGRHVLLYAHRISPEQI | |
| GI: 71059831 | DTVGIYGKVNIHSIGFRFSSDLQSMETSALGLTQINRENIQKPGK | |
| | LQLSLPFEARLNASMGPGRTVVIKGEVNTNARSFNVDLVAGKT | |
| | RDIALHLNPRLNVKAFVRNSFLQDAWGEEERNITCFPFSSGMYF | |
| | EMIIYCDVREFKVAINGVHSLEYKHRFKDLSSIDTLSVDGDIRLL | |
| | DVRSW | |
| Lgals8 | MMLSLNNLQNIIYNPVIPYVGTIPDQLDPGTLIVICGHVPSDADR | 10 |
| (Human) | FQVDLQNGSSVKPRADVAFHFNPRFKRAGCIVCNTLINEKWGR | |
| AAF19370.1 | EEITYDTPFKREKSFEIVIMVLKDKFQVAVNGKHTLLYGHRIGPE | |
| GI: 6625728 | KIDTLGIYGKVNIHSIGFSFSSDLQSTQASSLELTEISRENVPKSGT | |
| | PQLSLPFAARLNTPMGPGRTVVVKGEVNANAKSFNVDLLAGKS | |
| | KDIALHLNPRLNIKAFVRNSFLQESWGEEERNITSFPFSPGMYFE | |
| | MIIYCDVREFKVAVNGVHSLEYKHRFKELSSIDTLEINGDIHLLE | |
| | VRSW | |
| Vps35 | MPTTQQSPQDEQEKLLDEAIQAVKVQSFQMKRCLDKNKLMDA | 11 |
| (Mouse) | LKHASNMLGELRTSMLSPKSYYELYMAISDELHYLEVYLTDEF | |
| AAG40621.1 | AKGRKVADLYELVQYAGNIIPRLYLLITVGVVYVKSFPQSRKDI | |
| GI: 11875394 | LKDLVEMCRGVQHPLRGLFLRNYLLQCTRNILPDEGEPTDEETT | |
| | GDISDSMDFVLLNFAEMNKLWVRMQHQGHSRDREKRERERQE | |
| | LRILVGTNLVRLSQLEGVNVERYKQIVLTGILEQVVNCRDALAQ | |
| | EYLMECIIQVFPDEFHLQTLNPFLRACAELHQNVNVKNIIIALIDR | |
| | LALFAHREDGPGIPAEIKLFDIFSQQVATVIQSRQDMPSEDVVSL | |
| | QVSLINLAMKCYPDRVDYVDKVLETTVEIFNKLNLEHIATSSAV | |
| | SKELTRLLKIPVDTYNNILTVLKLKHFHPLFEYFDYESRKSMSCY | |
| | VLSNVLDYNTEIVSQDQVDSIMNLVSTLIQDQPDQPVEDPDPED | |
| | FADEQSLVGRFIHLLRSDDPDQQYLILNTARKHFGAGGNQRIRF | |
| | TLPPLVFAAYQLAFRYKENSQMDDKWEKKCQKIFSFAHQTISA | |
| | LIKAELAELPLRLFLQGALAAGEIGFENHETVAYEFMSQAFSLY | |
| | EDEISDSKAQLAAITLIIGTFERMKCFSEENHEPLRTQCALAASK | |
| | LLKKPDQGRAVSTCAHLFWSGRNTDKNGEELHGGKRVMECLK | |
| | KALKIANQCMDPSLQVQLFIEILNRYIYFYEKENDAVTIQVLNQL | |
| | IQKIREDLPNLESSEETEQINKHFHNTLEHLRSRRESPESEGPIYE | |
| | GLIL | |
| Vps35 | MPTTQQSPQDEQEKLLDEAIQAVKVQSFQMKRCLDKNKLMDA | 12 |
| (Human) | LKHASNMLGELRTSMLSPKSYYELYMAISDELHYLEVYLTDEF | |
| NP_060676.2 | AKGRKVADLYELVQYAGNIIPRLYLLITVGVVYVKSFPQSRKDI | |
| GI: 17999541 | LKDLVEMCRGVQHPLRGLFLRNYLLQCTRNILPDEGEPTDEETT | |
| | GDISDSMDFVLLNFAEMNKLWVRMQHQGHSRDREKRERERQE | |
| | LRILVGTNLVRLSQLEGVNVERYKQIVLTGILEQVVNCRDALAQ | |
| | EYLMECIIQVFPDEFHLQTLNPFLRACAELHQNVNVKNIIIALIDR | |
| | LALFAHREDGPGIPADIKLFDIFSQQVATVIQSRQDMPSEDVVSL | |
| | QVSLINLAMKCYPDRVDYVDKVLETTVEIFNKLNLEHIATSSAVV | |
| | SKELTRLLKIPVDTYNNILTVLKLKHFHPLFEYFDYESRKSMSCY | |
| | VLSNVLDYNTEIVSQDQVDSIMNLVSTLIQDQPDQPVEDPDPED | |
| | FADEQSLVGRFIHLLRSEDPDQQYLILNTARKHFGAGGNQRIRF | |
| | TLPPLVFAAYQLAFRYKENSKVDDKWEKKCQKIFSFAHQTISAL | |
| | IKAELAELPLRLFLQGALAAGEIGFENHETVAYEFMSQAFSLYE | |
| | DEISDSKAQLAAITLIIGTFERMKCFSEENHEPLRTQCALAASKL | |
| | LKKPDQGRAVSTCAHLFWSGRNTDKNGEELHGGKRVMECLKK | |
| | ALKIANQCMDPSLQVQLFIEILNRYIYFYEKENDAVTIQVLNQLI | |
| | QKIREDLPNLESSEETEQINKHFHNTLEHLRLRRESPESEGPIYEG | |
| | LIL | |

### REFERENCES

The following publications are herein incorporated by reference to the same extent as if each individual publication was specifically and individually indicated to be incorporated by reference.
1. Lu, H et al “The tumor antigen repertoire identified in tumor-bearing neu transgenic mice predicts human tumor antigens.” Cancer Res. 66(19): 9754-9761 (2006).
2. Disis, M et al “High-titer HER-2/neu protein-specific antibody can be detected in patients with early-stage breast cancer” J. Clin Oncol. 15(11): 3363-3367 (1997).
3. Herschkowitz, J et al. “Identification of conserved gene features between murine mammary carcinoma models and human breast tumors.” Genome Biol. 8(5): R76 (2007).
4. Muggerd A et al. “Molecular diversity in ductal carcinoma in situ (DCIS) and early invasive breast cancer.” Mol. Oncol. 4(4): 357-368 (2010).
5. Disis, M et al “Generation of T-cell immunity to the Her-2neu protein after active immunization with Her-2/neu peptide-based vaccines.” J. Clin Oncol 20(11): 2624-2632 (2002).
6. Curigliano et al “Breast cancer vaccines: a clinical reality or fairy tale?” Ann One 17(5): 750-762 (2006).
7. Mittendorf et al “Breast cancer vaccines: promise for the future or pipe dream” Cancer 110(8): 1677-1686 (2007).
8. Zaemler, P and Ziman, M “Serological autoantibodies as cancer biomarkers—a review” Cancer Epi Biomarkers and Prevention 22(12): 2061-2081 (2013).
9. Lu, H et al “Evaluation of known oncoantibodies Her2, p53, and Cyclin B1 in prediagnostic breast cancer sera.” Cancer Prevention Research. 5(8): 1036-1043 (2012).
10. Tan, H et al “Serum autoantibodies as biomarkers for early cancer detection.” FEBS J. 276(23): 6880-6904 (2009).
11. Anderson G L, Manson J, Wallace R, Lund B, Hall D, Davis S, Shumaker S, Wang C Y, Stein E, Prentice R L: Implementation of the Women's Health Initiative study design. Ann Epidemiol 2003, 13:S5-S17.
12. Lu H, Goodell V, Disis M L: Humoral immunity directed against tumor-associated antigens as potential biomarkers for the early diagnosis of cancer. J Proteome Res 2008, 7:1388-1394.
13. Qiu J, Madoz-Gurpide J, Misek D E, Kuick R, Brenner D E, Michailidis G, Haab B B, Omenn G S, Hanash S: Development of natural protein microarrays for diagnosing cancer based on an antibody response to tumor antigens. J Proteome Res 2004, 3:261-267.
14. Chapman C, Murray A, Chakrabarti J, Thorpe A, Woolston C, Sahin U, Barnes A, Robertson J: Autoantibodies in breast cancer: their use as an aid to early diagnosis. Ann Oncol 2007, 18:868-873.
15. Mange A, Lacombe J, Bascoul-Mollevi C, Jarlier M, Lamy P J, Rouanet P, Maudelonde T, Solassol J: Serum autoantibody signature of ductal carcinoma in situ progression to invasive breast cancer. Clin Cancer Res 2012, 18:1992-2000.
16. Anderson K S, Sibani S, Wallstrom G, Qiu J, Mendoza E A, Raphael J, Hainsworth E, Montor W R, Wong J, Park J G, Lokko N, Logvinenko T, Ramachandran N, Godwin A K, Marks J, Engstrom P, Labaer J: Protein microarray signature of autoantibody biomarkers for the early detection of breast cancer. J Proteome Res 2011, 10:85-96.
17. Desmetz C, Bascoul-Mollevi C, Rochaix P, Lamy P J, Kramar A, Rouanet P, Maudelonde T, Mange A, Solassol J: Identification of a new panel of serum autoantibodies associated with the presence of in situ carcinoma of the breast in younger women. Clin Cancer Res 2009, 15:4733-4741.
18. Yeaman S J, Kirby J A, Jones D E: Autoreactive responses to pyruvate dehydrogenase complex in the pathogenesis of primary biliary cirrhosis. Immunol Rev 2000, 174:238-249.
19. Thurston T L, Wandel M P, von Muhlinen N, Foeglein A, Randow F: Galectin 8 targets damaged vesicles for autophagy to defend cells against bacterial invasion. Nature 2012, 482:414-418.
20. Massardo L1, Metz C, Pardo E, Mezzano V, Babul M, Jarpa E, Guzman A M, Andrd S, Kaltner H, Gabius H J, Jacobelli S, GonzAlez A, Soza A: Autoantibodies against galectin-8: their specificity, association with lymphopenia in systemic lupus erythematosus and detection in rheumatoid arthritis and acute inflammation. Lupus 2009, 18:539-546.
21. Eshkar Sebban L, Ronen D, Levartovsky D, Elkayam O, Caspi D, Aamar S, Amital H, Rubinow A, Golan I, Naor D, Zick Y: The involvement of CD44 and its novel ligand galectin-8 in apoptotic regulation of autoimmune inflammation. J Immunol 2007, 179:1225-1235.
22. Xu Z, Zheng Y, Zhu Y, Kong X, Hu L: Evidence for OTUD-6B participation in B lymphocytes cell cycle after cytokine stimulation. PLoS One 2011, 6:e14514.
23. Wang Y1. O'Connell J R, McArdle P F, Wade J B, Dorff S E, Shah S J, Shi X, Pan L, Rampersaud E, Shen H, Kim J D, Subramanya A R, Steinle N I, Parsa A, Ober C C, Welling P A, Chakravarti A, Weder A B, Cooper R S, Mitchell B D, Shuldiner A R, Chang Y P: From the Cover: Whole-genome association study identifies STK39 as a hypertension susceptibility gene. Proc Natl Acad Sci USA 2009, 106:226-231.
24. Okado H1, Ohtaka-Maruyama C, Sugitani Y, Fukuda Y, Ishida R, Hirai S, Miwa A, Takahashi A, Aoki K, Mochida K, Suzuki O, Honda T, Nakajima K, Ogawa M, Terashima T, Matsuda J, Kawano H, Kasai M: The transcriptional repressor RP58 is crucial for cell-division patterning and neuronal survival in the developing cortex. Dev Biol 2009, 331:140-151.
25. Zimprich A1, Benet-Pagés A, Struhal W, Graf E, Eck S H, Offman M N, Haubenberger D, Spielberger S, Schulte E C, Lichtner P, Rossle S C, Klopp N, Wolf E, Seppi K, Pirker W, Presslauer S, Mollenhauer B, Katzenschlager R, Foki T, Hotzy C, Reinthaler E, Harutyunyan A, Kralovics R, Peters A, Zimprich F, Bricke T, Poewe W, Auff E, Trenkwalder C, Rost B, Ransmayr G, Winkelmann J, Meitinger T, Strom T M: A mutation in VPS35, encoding a subunit of the retromer complex, causes late-onset Parkinson disease. Am J Hum Genet 2011, 89:168-175.
26. Grivennikov S I, Greten F R, Karin M: Immunity, inflammation, and cancer. Cell 2010, 140:883-899.
27. Baumgarth N: A two-phase model of B-cell activation. Immunol Rev 2000, 176:171-180.
28. Gad, E., L, Rastetter, M, Slota, M, Koehnlein, P. M. Treuting, Y. Dang, et al., Natural history of tumor growth and immune modulation in common spontaneous murine mammary tumor models. Breast Cancer Res Treat. 2014, 148(3): p. 501-10.
29. Xu, Z., Y. Zheng. Y. Zhu, X. Kong, and L. Hu. Evidence for OTUD-6B participation in B lymphocytes cell cycle after cytokine stimulation. PLoS One. 2011, 6(1): p. e14514.
30. Jarvinen, A. K., R. Autio, S. Kilpinen, M. Saarela, I. Leivo, R. Grenman, et al., High-resolution copy number and gene expression microarray analyses of head and neck squamous cell carcinoma cell lines of tongue and larynx. Genes Chromosomes Cancer, 2008, 47(6): p. 500-9.
31. Chen, B., Y. Liu, X. Jin, W. Lu, J. Liu, Z. Xia, et al., MicroRNA-26a regulates glucose metabolism by direct targeting PDHX in colorectal cancer cells. BMC Cancer, 2014, 14: p. 443.
32. Huang, Y. T., R. S. Heist, L. R. Chirieac, X. Lin, V. Skaug, S. Zienolddiny, et al., Genome-wide analysis of survival in early-stage non-small-cell lung cancer. J Clin Oncol, 2009, 27(16): p. 2660-7.
33. Mao, J., J. Ladd, E. Gad, L. Rastetter, M. M. Johnson, E. Marzbani, et al., Mining the pre-diagnostic antibody repertoire of TgMMTV-neu mice to identify autoantibodies useful for the early detection of human breast cancer. J Transl Med. 2014, 12: p. 121.

1-54. (canceled)

55. A method for identifying a subject's risk for developing breast cancer, the method comprising:
a) incubating a biological sample from the subject with at least a first probe, wherein the biological sample comprises an autoantibody selected from Otud6B, Pdhx, Stk39, or a combination thereof and the first probe comprises a first recombinant polypeptide comprising an antigen of Otud6B, Pdhx, Stk39, or a combination thereof;
b) forming a first autoantibody-probe complex comprising the autoantibody and the first probe of step a);
c) measuring the concentration of the first autoantibody-probe complex, thereby determining the concentration of the autoantibody; and
d) identifying the subject as at risk for developing breast cancer if the subject has an elevated concentration of the autoantibody relative to a control.

56. The method of claim 55, wherein the control is a biological sample obtained from a subject who is not at risk for developing breast cancer.

57. The method of claim 55, wherein the first recombinant polypeptide is a polypeptide comprising at least 85% sequence identity to SEQ ID NO: 1 (Otud6B), at least 85% sequence identity to SEQ ID NO: 3 (Pdhx), or at least 85% sequence identity to SEQ ID NO: 5 (Stk39).

58. The method of claim 55, wherein the autoantibody is an IgG or an IgM autoantibody.

59. The method of claim 55, wherein the subject is at risk for developing ductal carcinoma in situ, lobular carcinoma in situ, invasive ductal carcinoma, infiltrating ductal carcinoma, inflammatory breast cancer, triple-negative breast cancer, paget disease of the nipple, phyllodes tumor, angiosarcoma, adenoid cystic carcinoma, adenocystic carcinoma, low-grade adenosquamous carcinoma, medullary carcinoma, mucinous carcinoma, colloid carcinoma, papillary carcinoma, tubular carcinoma, metaplastic carcinoma, spindle cell carcinoma, squamous carcinoma, micropapillary carcinoma, or mixed carcinoma.

60. The method of claim 55, wherein the subject is at risk for developing invasive breast cancer or at risk for developing ductal carcinoma in situ.

61. The method of claim 55, further comprising administering to the subject a composition comprising:
i) an isolated and purified plasmid comprising at least one nucleotide sequence encoding a polypeptide comprising at least 70% sequence identity to an epitope sequence of SEQ ID NO: 1 (Otud6B), SEQ ID NO: 3 (Pdhx), or SEQ ID NO: 5 (Stk39); or
ii) a nucleic acid polymer that hybridizes to a target sequence encoding Otud6B, Pdhx, or Stk39, wherein the nucleic acid polymer modulates the gene expression of the target sequence;
thereby reducing the risk of or preventing breast cancer in the subject.

62. The method of claim 55, wherein the method comprises:
a) incubating a biological sample from the subject with a panel of probes, wherein the biological sample comprises at least one autoantibody selected from Otud6B, Pdhx, Stk39, or a combination thereof and each probe from the panel of probes comprises a recombinant polypeptide comprising an antigen of Otud6B, Pdhx, Stk39, or a combination thereof;
b) forming at least one autoantibody-probe complex comprising the at least one autoantibody and the probe of step a);
c) measuring the concentration of the at least one autoantibody-probe complex, thereby determining the concentration of the at least one autoantibody; and
d) identifying the subject as at risk for developing breast cancer if the subject has an elevated concentration of the at least one autoantibody relative to a control.

63. A method of prevention or treatment of breast cancer, comprising administering to a subject in need thereof a composition comprising:
a) an isolated and purified plasmid comprising at least one nucleotide sequence encoding a polypeptide comprising at least 70% sequence identity to an epitope sequence of SEQ ID NO: 1, SEQ ID NO: 3, or SEQ ID NO: 5; or
b) a nucleic acid polymer that hybridizes to a target sequence encoding Otud6B, Pdhx, or Stk39, wherein the nucleic acid polymer modulates gene expression of the target sequence.

64. The method of claim 63, wherein administration of the composition comprising the isolated and purified plasmid reduces or inhibits tumor growth.

65. The method of claim 63, wherein the nucleic acid polymer decreases gene expression of the target sequence.

66. The method of claim 65, wherein a decrease in the gene expression of the target sequence leads to an increase in apoptosis of tumor cells.

67. The method of claim 63, wherein the breast cancer is ductal carcinoma in situ, lobular carcinoma in situ, invasive ductal carcinoma, infiltrating ductal carcinoma, inflammatory breast cancer, triple-negative breast cancer, paget disease of the nipple, phyllodes tumor, angiosarcoma, adenoid cystic carcinoma, adenocystic carcinoma, low-grade adenosquamous carcinoma, medullary carcinoma, mucinous carcinoma, colloid carcinoma, papillary carcinoma, tubular carcinoma, metaplastic carcinoma, spindle cell carcinoma, squamous carcinoma, micropapillary carcinoma, or mixed carcinoma.

68. The method of claim 63, wherein the subject has invasive breast cancer or ductal carcinoma in situ.

69. The method of claim 63, wherein the composition is formulated for subcutaneous, intramuscular, or intradermal administration.

70. The method of claim 63, wherein the composition is administered in combination with an additional therapeutic agent.

71. The method of claim 70, wherein the additional therapeutic agent comprises checkpoint inhibitors, costimulatory molecules, immune-cellular, or -intracellular targeting molecules, or combinations thereof.

72. A composition comprising:
a) an isolated and purified plasmid comprising at least one nucleotide sequence encoding a polypeptide comprising at least 70% sequence identity to an epitope sequence of SEQ ID NO: 1, SEQ ID NO: 3, or SEQ ID NO: 5; and
b) an excipient and/or a carrier.

73. The composition of claim 72, further comprising an adjuvant.

74. The composition of claim 72, wherein the composition is formulated for subcutaneous, intramuscular, or intradermal administration.


SEQUENCE LISTING (12 sequences)

SEQ ID NO: 1 - protein, 293 aa, Homo sapiens
MEAVLTEELDEEEQLLRRHRKEKKELQAKIQGMKNAVPKNDKKRRKQLTEDVAKLEKEMEQKHREELEQLKLTTKENKIDSVAVNISNLVLENQPPRISKAQKRREKKAALEKEREERIAEAEIENLTGARHMESEKLAQILAARQLEIKQIPSDGHCMYKAIEDQLKEKDCALTVVALRSQTAEYMQSHVEDFLPFLTNPNTGDMYTPEEFQKYCEDIVNTAAWGGQLELRALSHILQTPIEIIQADSPPIIVGEEYSKKPLILVYMRHAYGLGEHYNSVTRLVNIVTENCS

SEQ ID NO: 2 - protein, 294 aa, Mus musculus
MEEVVAEELDDEEQLVRRHRKEKKELQAKIQGMKNAVPKNDKKRRKQLTEDVAKLEREMEQKHREELEQLKQLTFKDSKIDSVAVNISNLVLENQPPRISKAQKRREKKAALEKEREERIAEAEIENLSGARHLESEKLAQILAARELEIKQIPSDGHCMYGALEDQLREQDCALTVASLRRQTAEYMQTHSDDFLPFLTNPSTGDMYTPEEFGKYCDDIVNTAAWGGQLELRALSHILQTPIEILQADAPPIIVGEEYPRNPLVLVYMRHAYGLGEHYNSVTRLVNSATENCS

SEQ ID NO: 3 - protein, 501 aa, Homo sapiens
MAASWRLGCDPRLLRYLVGFPGRRSVGLVKGALGWSVSRGANWRWFHSTQWLRGDPIKILMPSLSPTMEEGNIVKWLKKEGEAVSAGDALCEIETDKAVVTLDASDDGILAKIVVEEGSKNIRLGSLIGLIVEEGEDWKHVEIPKDVGPPPPVSKPSEPRPSPEPQISIPVKKEHIPGTLRFRLSPAARNILEKHSLDASQGTATGPRGIFTKEDALKLVQLKQTGKITESRPTPAPTATPTAPSPLQATAGPSYPRPVIPPVSTPGQPNAVGTFTEIPASNIRRVIAKRLTESKSTVPHAYATADCDLGAVLKVRQDLVKDDIKVSVNDFIIKAAAVTLKQMPDVNVSWDGEGPKQLPFIDISVAVATVKGLLTPIIKDAAAKGIQEIADSVKALSKKARDGKLLPEEYQGGSFSISNLGMFGIDEFTAVINPPQACILAVGRFRPVLKLTEDEEGNAKLQQRQLITVTMSSDSRVVDDELATRFLKSFKANLENPIRLA

SEQ ID NO: 4 - protein, 501 aa, Mus musculus
MAASWRLHCNQPLLRYLLGFSSRRSLGLAQGAAAWPVDRGASWRWFHSTQLLQADPIKVLMPSLSPTMEQGNIVKWLRKEGEAVSAGDSLCEIETDKAVVTLDANDDGILAKIVVEEGAKNIQLGSLIALMVEEGEDWKQVEIPKDVSAPPPVSKPPAPTQPSPQPQIPCPARKEHKGTARFRLSPAARNILEKHSLDASQGTATGPRGIFTKEDALKLVELKQMGKITESRPASAPPPSLSASVPPQATAGPSYPRPMTPPVSIPGQPNAAGTFTEIPASNIRRVIAKRLTESKSTVPHAYATADCDLGAVLKVRRDLVKDDIKVSVNDFIIRAAAVTLKQMPGVNVTWDGEGPKQLPSVDISVAVATDKGLITPIIKDAAAKGIQEIADSVKVLSKKARDGKLMPEEYQGGSFSISNLGMFGIDEFAAVINPPQACILAVGRFRPVLKLTEDEEGNPQLQQHQLITVTMSSDSRVVDDELATRFLETFKANLENPMRLG

SEQ ID NO: 5 - protein, 545 aa, Homo sapiens
MAEPSGSPVHVQLPQQAAPVTAAAAAAPAAATAAPAPAAPAAPAPAPAPAAQAVGWPICRDAYELQEVIGSGATAVVQAALCKPRQERVAIKRINLEKCQTSMDELLKEIQAMSQCSHPNVVTYYTSFVVKDELWLVMKLLSGGSMLDIIKYIVNRGEHKNGVLEEAIIATILKEVLEGLDYLHRNGQIHRDLKAGNILLGEDGSVQIADFGVSAFLATGGDVTRNKVRKTFVGTPCWMAPEVMEQVRGYDFKADMWSFGITAIELATGAAPYHKYPPMKVLMLTLQNDPPTLETGVEDKEMMKKYGKSFRKLLSLCLQKDPSKRPTAAELLKCKFFQKAKNREYLIEKLLTRTPDIAQRAKKVRRVPGSSGHLHKTEDGDWEWSDDEMDEKSEEGKAAFSQEKSRRVKEENPEIAVSASTIPEQIQSLSVHDSQGPPNANEDYREASSCAVNLVLRLRNSRKELNDIRFEFTPGRDTADGVSQELFSAGLVDGHDVVIVAANLQKIVDDPKALKTLTFKLASGCDGSEIPDEVKLIGFAQLSVS

SEQ ID NO: 6 - protein, 556 aa, Mus musculus
MAEPSGSPVHVQLSQQAAPVTAAAATAPAAATSAPAPAPAPAPAASAAPAPAPAAAPAPAPAAQAVGWPICRDAYELQEVIGSGATAVVQAALCKPRQERVAIKRINLEKCQTSMDELLKEIQAMSQCSHPNVVTYYTSFVVKDELWLVMKLLSGGSMLDIIKYIVNRGEHKNGVLEEAIIATILKEVLEGLDYLHRNGQIHRDLKAGNILLGEDGSVQIADFGVSAFLATGGDVTRNKVRKTFVGTPCWMAPEVMEQVRGYDFKADMWSFGITAIELATGAAPYHKYPPMKVLMLTLQNDPPTLETGVEDKEMMKKYGKSFRKLLSLCLQKDPSKRPTAAELLKCKFFQKAKNREYLIEKLLTRTPDIAQRAKKVRRVPGSSGHLHKTEDGDWEWSDDEMDEKSEEGKAAASQEKSRRVKEENSEISVNAGGIPEQIQSLSVHDSQAQPNANEDYREGPCAVNLVLRLRNSRKELNDIRFEFTPGRDTADGVSQELFSAGLVDGHDVVIVAANLQKIVDDPKALKTLTFKLASGCDGSEIPDEVKLIGFAQLSVS

SEQ ID NO: 7 - protein, 522 aa, Mus musculus
MEFPDHSRHLLQCLSEQRHQGFLCDCTVLVGDAQFRAHRAVVASCSMYFHLFYKDQLDKRDIVHLNSDIVTAPAFALLLEFMYEGKLQFKDLPIEDVLAAASYLHMYDIVKVCKKKLKEKATTEADSTKKEEDASSCSDKVESLSDGSSHMAGDLPSDEDEGEDDKLNILPSKRDLAAEPGNMWMRLPSDSAGIPQAGGEAEPHATAAGKTVASPCSSTESLSQRSVTSVRDSADVDCVLDLSVKSSLSGVENLNSSYFSSQDVLRSNLVQVKVEKEASCDESDVGTNDYDMEHSTVKESVSTNNRVQYEPAHLAPLREDSVLRELDREDKASDDEMMTPESERVQVEGGMENSLLPYVSNILSPAGQIFMCPLCNKVFPSPHILQIHLSTHFREQDGIRSKPAADVNVPTCSLCGKTFSCMYTLKRHERTHSGEKPYTCTQCGKSFQYSHNLSRHAVVHTREKPHACKWCERRFTQSGDLYRHIRKFHCELVNSLSVKSEALSLPTVRDWTLEDSSQELWK

SEQ ID NO: 8 - protein, 531 aa, Homo sapiens [sequence omitted]

SEQ ID NO: 9 - protein, 316 aa, Mus msuculus
MLSLNNLQNIIYNPIIPYVGTITEQLKPGSLIVIRGHVPKDSERFQVDFQLGNSLKPRADVAFHFNPRFKRSSCIVCNTLTQEKWGWEEITYDMPFRKEKSFEIVFMVLKNKFQVAVNGRHVLLYAHRISPEQIDTVGIYGKVNIHSIGFRFSSDLQSMETSALGLTQINRENIQKPGKLQLSLPFEARLNASMGPGRTVVIKGEVNTNARSFNVDLVAGKTRDIALHLNPRLNVKAFVRNSFLQDAWGEEERNITCFPFSSGMYFEMIIYCDVREFKVAINGVHSLEYKHRFKDLSSIDTLSVDGDIRLLDVRSW

SEQ ID NO: 10 - protein, 317 aa, Homo sapiens
MMLSLNNLQNIIYNPVIPYVGTIPDQLDPGTLIVICGHVPSDADRFQVDLQNGSSVKPRADVAFHFNPRFKRAGCIVCNTLINEKWGREEITYDTPFKREKSFEIVIMVLKDKFQVAVNGKHTLLYGHRIGPEKIDTLGIYGKVNIHSIGFSFSSDLQSTQASSLELTEISRENVPKSGTPQLSLPFAARLNTPMGPGRTVVVKGEVNANAKSFNVDLLAGKSKDIALHLNPRLNIKAFVRNSFLQESWGEEERNITSFPFSPGMYFEMIIYCDVREFKVAVNGVHSLEYKHRFKELSSIDTLEINGDIHLLEVRSW

SEQ ID NO: 11 - protein, 796 aa, Mus musculus
MPTTQQSPQDEQEKLLDEAIQAVKVQSFQMKRCLDKNKLMDALKHASNMLGELRTSMLSPKSYYELYMAISDELHYLEVYLTDEFAKGRKVADLYELVQYAGNIIPRLYLLITVGVVYVKSFPQSRKDILKDLVEMCRGVQHPLRGLFLRNYLLQCTRNILPDEGEPTDEETTGDISDSMDFVLLNFAEMNKLWVRMQHQGHSRDREKRERERQELRILVGTNLVRLSQLEGVNVERYKQIVLTGILEQVVNCRDALAQEYLMECIIQVFPDEFHLQTLNPFLRACAELHQNVNVKNIIIALIDRLALFAHREDGPGIPAEIKLFDIFSQQVATVIQSRQDMPSEDVVSLQVSLINLAMKCYPDRVDYVDKVLETTVEIFNKLNLEHIATSSAVSKELTRLLKIPVDTYNNILTVLKLKHFHPLFEYFDYESRKSMSCYVLSNVLDYNTEIVSQDQVDSIMNLVSTLIQDQPDQPVEDPDPEDFADEQSLVGRFIHLLRSDDPDQQYLILNTARKHFGAGGNQRIRFTLPPLVFAAYQLAFRYKENSQMDDKWEKKCQKIFSFAHQTISALIKAELAELPLRLFLQGALAAGEIGFENHETVAYEFMSQAFSLYEDEISDSKAQLAAITLIIGTFERMKCFSEENHEPLRTQCALAASKLLKKPDQGRAVSTCAHLFWSGRNTDKNGEELHGGKRVMECLKKALKIANQCMDPSLQVQLFIEILNRYIYFYEKENDAVTIQVLNQLIQKIREDLPNLESSEETEQINKHFHNTLEHLRSRRESPESEGPIYEGLIL

SEQ ID NO: 12 - protein, 796 aa, Homo sapiens
MPTTQQSPQDEQEKLLDEAIQAVKVQSFQMKRCLDKNKLMDALKHASNMLGELRTSMLSPKSYYELYMAISDELHYLEVYLTDEFAKGRKVADLYELVQYAGNIIPRLYLLITVGVVYVKSFPQSRKDILKDLVEMCRGVQHPLRGLFLRNYLLQCTRNILPDEGEPTDEETTGDISDSMDFVLLNFAEMNKLWVRMQHQGHSRDREKRERERQELRILVGTNLVRLSQLEGVNVERYKQIVLTGILEQVVNCRDALAQEYLMECIIQVFPDEFHLQTLNPFLRACAELHQNVNVKNIIIALIDRLALFAHREDGPGIPADIKLFDIFSQQVATVIQSRQDMPSEDVVSLQVSLINLAMKCYPDRVDYVDKVLETTVEIFNKLNLEHIATSSAVSKELTRLLKIPVDTYNNILTVLKLKHFHPLFEYFDYESRKSMSCYVLSNVLDYNTEIVSQDQVDSIMNLVSTLIQDQPDQPVEDPDPEDFADEQSLVGRFIHLLRSEDPDQQYLILNTARKHFGAGGNQRIRFTLPPLVFAAYQLAFRYKENSKVDDKWEKKCQKIFSFAHQTISALIKAELAELPLRLFLQGALAAGEIGFENHETVAYEFMSQAFSLYEDEISDSKAQLAAITLIIGTFERMKCFSEENHEPLRTQCALAASKLLKKPDQGRAVSTCAHLFWSGRNTDKNGEELHGGKRVMECLKKALKIANQCMDPSLQVQLFIEILNRYIYFYEKENDAVTIQVLNQLIQKIREDLPNLESSEETEQINKHFHNTLEHLRLRRESPESEGPIYEGLIL